SEQUENCE LISTING (27 sequences)

SEQ ID NO: 1 - DNA, 2343 bp, Artificial Sequence
  DENV2 CprME nucleic acid sequence human codon optimized
gctagcgccatgaacaaccagcggaagaaggcccggaacacccctttcaacatgctgaagcgcgagcggaacagagtgtccacagtgcagcagctgaccaagagattcagcctgggcatgctgcaaggcagaggccctctgaagctgtttatggccctggtggccttcctgcggttcctgacaattcctccaacagccggcatcctgaagagatggggcaccatcaagaagtccaaggccatcaacgtgctgcggggcttcagaaaagaaatcggccggatgctgaacatcctgaaccggcggagaagaaccgccggcatcattatcatgatgatccccaccgtgatggctttccacctgaccaccagaaacggcgagccccacatgatcgtgtccagacaagagaagggcaagagcctgctgttcaagaccgagaacggcgtgaacatgtgcaccctgatggccatggatctgggcgagctgtgcgaggacaccatcacctacaattgccctctgctgaagcagaacgagcccgaggacatcgactgctggtgtaacagcaccagcacctgggtcacctacggcacctgtacagccacaggcgaacacagacgcgagaagagatctgtggctctggtgcctcacgttggcatgggcctcgagacaagaaccgagacatggatgtctagcgaaggcgcctggaagcacgcccagagaattgagacatgggtgctgagacaccccggcttcacagtgatggccgccatcctggcctacaccatcggcaccacctacttccagcgggtgctgatcttcatcctgctgacagccgtggctcccagcatgaccatgagatgcatcggcatcagcaaccgggacttcgtggaaggtgttagcggcggctcttgggtcgacatcgtgctggaacacggcagctgtgtgaccaccatggccaagaacaagcccacactggacttcgagctgatcaagacagaggctaagcaccccgccacactgcggaagtactgtatcgaggccaagctgacaaacaccaccaccgccagcagatgccctacacagggcgagcctagcctgaacgaggaacaggataagagattcgtgtgcaagcacagcatggtggatagaggctggggcaatggctgtggcctgtttggcaaaggcggcatcgtgacctgcgccatgttcacctgtaaaaagaacatggaaggcaaggtggtgcagcccgagaacctcgagtacacaatcgtgatcacccctcacagcggcgaagagaacgccgtgggaaatgatacaggcaagcacggcaaagagatcaaagtcaccccacagagcagcatcaccgaggccgaactgacaggctacggcacagtgaccatggaatgctccccaagaaccggcctggacttcaacgagatggtgctgctgcagatggaaaacaaggcctggctggtgcaccggcagtggtttctggatctgcctcttccttggctgcctggcgctgatacccagggctctaactggattcagaaagagacactggtcaccttcaagaaccctcacgccaagaaacaggacgtggtggtgctgggatctcaagagggcgccatgcatacagctctgacaggcgccacagagatccagatgagcagcggcaacctgctgtttaccggccacctgaagtgccggctgagaatggacaaactgcagctgaagggcatgagctacagcatgtgtaccggcaagttcaaggtcgtgaaagagatcgccgagacacagcacggcaccatcgtgatccgggtgcagtatgaaggcgacggcagcccttgcaagatccccttcgagatcatggacctggaaaagcggcacgtgctgggcagactgatcaccgtgaatcctatcgtgaccgagaaggacagccccgtgaacattgaagccgagcctccattcggcgacagctacatcatcatcggcgtggaacctggccagctgaagctgagctggttcaagaagggcagcagcatcggccagatgttcgagacaacaatgagaggcgccaagcggatggccattctgggcgatacagcctgggattttggctctctcggcggcgtgttcacctctatcggaaaagccctgcaccaggtgttcggcgccatctatggcgctgcctttagcggagtgtcctggaccatgaagatcctgatcggcgttgtgatcacctggatcggcatgaactccagatccaccagcctgagcgtgtccctggtgcttgtgggagtcgtgacactgtacctgggcgtgatggttcaggcttgagaattc

SEQ ID NO: 2 - DNA, 7948 bp, Artificial Sequence
  DENV2 NS1-5 nucleic acid sequence human codon optimized
gggctagcgccaccatgagcacaagcctgtctgtgtctctggtgctcgtgggcgtcgtgacactgtatctgggagtgatggtgcaggccgatagcggctgtgtggtgtcctggaagaacaaagaactgaagtgcggcagcggcatcttcatcaccgacaacgtgcacacctggaccgagcagtacaagttccagcctgagagccctagcaagctggcctctgccattcagaaggcccacgaggaaggcatctgcggcatcagaagcgtgacccggctggaaaacctgatgtggaagcagatcacccctgagctgaaccacatcctgagcgagaacgaagtgaagatgaccatcatgaccggcgacatcaagggcatcatgcaggccggcaaaagaagcctgaggccacagcctaccgagctgaagtactcttggaaggcctggggcaaagccaagatgctgagcaccgagctgcacaaccacacctttctgatcgacggccccgagacagccgagtgtcccaataccaacagagcctggaacagcctggaagtggaagattacggcttcggcgtgttcaccaccaacatctggctgaagctgaaagagcggcaggacgtttcctgcgacagcaagctgatgagcgccgccatcaaggacaatagagccgtgcacgccgacatgggctactggattgagagcgccctgaacgacacctggaagatcgagaaggcctccttcatcgaagtgaaaagctgccactggcctaagagccacacactgtggtccaacggcgtgctggaaagcgagatgatcattcccaagaacttcgctggccccgtgtctcagcacaactacagacctggctaccacacacagaccgccggaccttggcatctgggcagactggaaatggacttcgacttctgcgagggcaccaccgtggtggtcacagaggattgtggcaacagaggccctagcctgagaaccacaacagccagcggaaagctgatcaccgagtggtgctgcagaagctgcacactgcctccactgagatacagaggcgaggacggctgttggtacggcatggaaatcagacccctgaaagagaaagaagagaacctggtcaacagcctcgtgacagccggacacggccagatcgacaatttttccctgggcgtgctcggcatggccctgtttctggaagaaatgctgcggaccagagtgggcaccaagcacgctattctgctggtggctgtgtccttcgtgaccctgatcacaggcaacatgagcttccgcgacctgggccgcgtgatggttatggttggagccacaatgaccgacgatatcggcatgggcgtgacctatctggctctgctggccgcttttaaagtgcggcctacatttgccgccggactgctgctgagaaagctgaccagcaaagaactcatgatgaccaccatcggcatcgtgctgctgtcccagagcacaatccccgaaaccatcctggaactgaccgacgctctggccctgggcatgatggtgctgaaggtcgtgcggaacatggaaaagtaccagctggccgtgacaatcatggccatcctgtgcgtgcccaacgccgtgatcctgcagaatgcctggaaggtgtcctgcaccactctggctgtggtgtcagtgtctcctctgctgctcaccagctctcagcagaaggccgattggatcccactggctctgaccatcaagggactgaaccctaccgccatcttcctgaccacactgagccggaccagcaagaagagatcttggcccctgaatgaggccatcatggccgtcggcatggtgtctattctggccagcagcctgctgaagaacgacatccctatgacaggccctctggtggcaggcggactgctcactgtgtgttatgtgctgacaggcagaagcgccgacctggaattggaaagggccgcagacgtccgctgggaagaacaggccgagatctctggcagctcccctatcctgagcatcaccatcagcgaggatggcagcatgagcatcaagaacgaggaagaggaacagatcctgaccatcctgatcagaaccggcctgctggtcatcagcggcctgtttccagtgtctatccccatcacagccgccgcttggtatctgtgggaagtcaagaaacagcgcgcaggcgtgctgtgggatgtgccttctcctccacctgtgggaaaagccgagctggaagatggggcctaccggatcaagcagaagggcatcctgggctactctcagattggagccggcgtgtacaaagagggcaccttccacacaatgtggcacgtgacaagaggcgccgtgctgatgcacaagggcaagagaatcgagcctagctgggccgatgtgaagaaggacctgatctcttacggcggaggctggaaactggaaggcgagtggaaagagggcgaagaggttcaggtgctggctctggaacccggcaagaatcctagagctgtgcagaccaagcctggcctgttcaagaccaacaccggcacaatcggagctgtgtccctggatttcagccctggcacatctggcagccccatcgtggacaagaaaggcaaggttgtgggcctgtacggcaatggcgtggtcacaagaagcggcacctacgtcagcgctatcgcccagaccgagaagtccatcgaggacaaccccgagatcgaggacgacatcttccggaaaaagcggctgaccattatggatctgcaccctggcgccggaaagaccaagagatatctgcccgccattgtgcgggaagccatcaagagaggactgcggaccctgattctggcccctacaagagtggtggccgccgaaatggaagaggctctgagaggcctgcctatcagataccagacaccagccatccgggccgaacacaccggaagagaaatcgtggatctgatgtgccacgccaccttcaccatgagactgctgagccccatcagagtgcccaactacaatctgatcatcatggacgaggcccacttcacagaccccgcctctattgccgccagaggctacatcagcacccgcgtggaaatgggagaagccgccggaatcttcatgaccgccacacctccaggctccagagatccatttcctcagagcaacgcccctatcatggatgaggaaagagagatccccgagcggagctggaatagcggacacgagtgggtcaccgacttcaagggaaagaccgtttggttcgtgcccagcatcaaggccggaaacgatatcgccgcctgcctgagaaagaacggcaagaaagtgatccagctgtcccgcaagaccttcgacagcgagtacatcaagacccggaccaacgactgggacttcgtggtcaccaccgatatctctgagatgggcgccaacttcaaggccgagagagtgatcgatcccagacggtgcatgaagcccgtgattctgaccgatggcgaagaaagagtgatcctggctggccccatgcctgtgacacattctagtgccgctcagcggagaggccgcgttggcagaaatcccaagaatgagagcgaccagtacatctacatgggcgagcccctggaaaacgacgaggattgcgcccactggaaagaagcaaaaatgctgctcgacaacatcaacacccctgaagggatcatccccagcatgttcgagcccgagcgcgagaaagtggacgccattgatggcgagtaccggctgagaggcgaagccagaaagacctttgtggacctgatgcggaggggcgatctgcctgtttggctggcctatagagtggccgctgagggcatcaactacgccgatagaagatggtgcttcgacggcgtgaagaacaaccagatcctcgaagagaacgtggaagtcgagatctggaccaaagaaggcgaacggaagaaactgaagccccggtggctggacgcccggatctattctgatcccctggcactgaaagagttcaaagagtttgccgctggccggaagtctctgaccctgaacctgattaccgagatggggagactgcccacctttatgacccagaaggccagaaacgccctggacaacctggccgtgctgcatacagctgaagcaggcggcagagcctacaatcacgccctgtctgagctgcccgagactctggaaactctgctcctgctgaccctgctggctacagtgaccggcggaatctttctgttcctgatgtccggcaaaggcatcggcaagatgacactgggcatgtgctgcatcatcaccgcctccatcctgctttggtacgcccagattcagccccactggatcgccgcttctattatcctggaattcttcctgatcgtcctgctgattcccgagccagagaagcagagaacccctcaggacaaccagctgacctacgtcgtgatcgccatcctgacagtggtggctgccacaatggccaacgagatgggcttcctggaaaagaccaaaaaggacttcggcctgggctccagcacaacccagcagcatgaatccaacatcctggacatcgacctgcggcctgcctctgcttggactctgtatgccgtggccaccaccttcatcacacccatgctgaggcacagcatcgagaacagcagcgtgaacgtgtccctgaccgcaatcgccaatcaggccacagtgctgatgggcctcggaaaaggatggcccctgtccaagatggatatcggcgttccactgctggcaatcggctgctacagccaagtgaaccccatcactctgacagccgcactgctgagtctggtggcccactatgccatcatcggacctggactgcaggccaaggccacaagagaagcccagaaaagagccgccgcaggcatcatgaagaaccccacagtggatggcatcaccgtgatcgacctggatcctattccttacgaccccaagttcgagaagcagctgggccaagtgatgctcctggtgctgtgtgtgacccaggtgctcatgatgcggacaacatgggccctgtgcgaagccctgacactggccacaggacctattagcactctgtgggagggcaaccccggcagattctggaataccacaatcgccgtgtccatggccaacatcttcagaggcagctacctggcaggcgctggactgctgttcagcattatgaagaataccgccaacaccagaagaggcaccggcaataccggcgagacactgggagagaagtggaaaaacagactgaacgctctgggcaagagcgagttccagatctacaagaagtccggcatccaagaggtggacagaaccctggccaaagagggaatcaagcggggcgagacagatcaccacgccgtgtctagaggatctgccaagctgcgttggttcgtcgagcggaatctggtcaccccagagggcaaagtggtggatctcggatgcggaagaggcggctggtcttactattgtggcggcctgaagaatgtgaaagaagtgaagggcctgaccaaaggcggacccggacacgaagaacccattcctatgtccacctacggctggaacctcgtgcgactgcagagcggagtggacgtgttctttacccctcctgagaagtgcgacaccctgctgtgcgatatcggagagagcagccccaatcctaccgtggaagccggcagaacactgagagtgctgaacctggtggaaaactggctgaacaacaacacccagttctgcatcaaggtgctcaacccctacatgcctagcgtgatcgagaaaatggaagccctgcagcggaagtatggcggagccctcgttagaaaccctctgagcagaaacagcacccacgagatgtattgggtgtccaacgcctccggcaacatcgtgtcctccgtgaacatgatctcccggatgctgatcaaccggttcaccatgcggcacaagaaggccacatacgagcccgatgtggatcttggctccggcaccagaaacatcggaatcgagagcgagacacccaacctggacattatcggcaagcggattgagaagatcaagcaagagcacgagacaagctggcactacgaccaggaccatccttacaagacatgggcctaccacggcagctacgagacaaagcagaccggcagcgccagctccatggttaatggcgttgtgcggctgctgaccaagccatgggatgtgatccctatggtcacccagatggctatgaccgacacaacccctttcggacagcagagggtgttcaaagaaaaggtggacaccagaacgcaagagcctaaagaaggcaccaagaaacttatgaagatcacggccgagtggctgtggaaagaactggggaagaaaaagacccctcggatgtgcacccgggaagagttcaccagaaaagtgcggagcaatgctgccctgggcgccatctttaccgacgagaacaagtggaagtccgccagagaggccgtcgaggattctggcttttgggagcttgtggacaaagagcggaacctgcacctcgagggcaagtgcgaaacctgtgtgtacaacatgatggggaagcgcgaaaagaagctgggcgagttcggaaaggccaagggcagcagagctatctggtacatgtggctgggagcccgctttctggaattcgaggctctgggctttctgaacgaggaccactggttcagcagagagaatagcctgagcggcgttgaaggcgagggactgcacaagctgggctatatcctgcgggacgtgtccaagaaagaaggcggcgctatgtacgccgatgataccgccggatgggataccagaatcaccctcgaggacctgaaaaacgaagagatggtcaccaaccacatggaaggggagcacaagaaactggccgaggccatcttcaagctgacataccagaacaaagtcgtgcgggtgcagcggccaacacctagaggaaccgtgatggacatcatcagcagacgggatcagcgcggctctggacaggttgtgacatacggcctgaacacctttaccaacatggaagctcagctgatccggcagatggaaggcgaaggcgtgttcaagagcatccagcagctgaccgctaccgaagagatcgccgtgaagaattggctcgttcgcgtgggcagagaacggctgtctagaatggctatcagcggcgacgactgcgtggtcaagcctctggacgacagatttgccagcgcactgaccgctctgaacgatatgggcaaagtccggaaggacattcagcagtgggagccctccagaggatggaacgattggactcaggtgccattctgctcccaccacttccacgagctgatcatgaaggacggcagagtgctggtggtgccttgccgcaatcaggatgagctgatcggacgcgccagaatctctcaaggtgccggatggtccctgagagagactgcctgtctgggcaaaagctacgcacagatgtggtcactgatgtacttccaccggcgggacctgagactggccgccaatgctatttgttccgccgtgccatctcactgggtgcccacaagcagaaccacctggtctatccacgccactcacgagtggatgaccacagaggacatgctgaccgtgtggaacagagtgtggatccaagagaacccttggatggaagataagaccccagtggaatcctgggaagagatcccttacctcggcaagagggaagatcagtggtgcggctctctgatcggcctgacctctagagccacatgggccaagaatatccagacggccatcaatcaagtgcgcagcctgatcggcaacgaagagtacaccgactacatgccctccatgaagcggttccgccgcgaagaggaagaggctggcgttctctggtagatgcatcc

SEQ ID NO: 3 - DNA, 7926 bp, Artificial Sequence
  DENV2 NS1-5 nucleic acid sequence old word monkey codon optimized
atgtctacttctttgagcgtgtccttggtgctggtgggagtggtgactttgtacttgggcgtgatggtgcaggccgattctggatgcgtggtgtcttggaaaaataaggagctgaaatgcgggtccggcatctttattaccgacaatgtgcatacctggaccgagcagtacaaatttcagcctgagtctccttccaaactggcctctgccatccagaaagcccacgaggagggcatctgcgggatcaggtctgtgacccgcctggagaatctgatgtggaaacagatcacccctgagctgaatcacatcctgtctgagaatgaggtgaaaatgaccatcatgaccggcgacatcaaaggcatcatgcaggccgggaaaagatccctgagacctcagcctaccgagctgaaatactcctggaaagcctggggcaaagccaaaatgctgtctaccgagctgcacaatcatacctttctgatcgacgggccggagaccgccgagtgccctaataccaatagagcctggaactctctggaggtggaggactacggctttggcgtgtttaccaccaatatctggctgaaactgaaagagcgccaggacgtgtcttgcgactccaaactgatgtccgccgccattaaggacaatagagccgtgcacgccgacatgggctactggattgagtctgccctgaatgacacctggaaaatcgagaaagcctcctttatcgaggtgaaatcttgccactggccgaaatctcataccctgtggtctaatggcgtgctggagtctgagatgattatccccaaaaattttgccggccccgtgtcccagcataattacaggcccggctaccatacccagaccgccggcccttggcatctggggagactggagatggactttgacttttgcgagggcaccaccgtggtggtgaccgaggactgcggcaatagagggccctccctgaggaccaccaccgcctccgggaaactgatcaccgagtggtgctgccgctcttgcaccctgcctccgctgagatacaggggcgaggacggctgctggtacgggatggagatcagacctctgaaagagaaagaggagaatctggtgaactccctggtgaccgccgggcacggccagattgacaatttttccctgggggtgctggggatggccctgtttctggaggagatgctgagaaccagggtggggaccaaacacgccattctgctggtggccgtgtcttttgtgaccctgattaccggcaatatgtcctttcgcgacctgggcagagtgatggtaatggtgggggccaccatgaccgacgacattgggatgggcgtgacctacctggccctgctggccgcctttaaagtgcgccctacctttgccgccgggctgctgctgagaaaactgacctccaaagagctgatgatgaccaccatcgggatcgtgctgctgtcccagtctaccatccccgagaccattctggagctgaccgacgccctggccctgggcatgatggtgctgaaagtggtgcgcaatatggagaaataccagctggccgtgaccatcatggccattctgtgcgtgccgaatgccgtgattctgcagaatgcctggaaagtgtcttgcaccaccctggccgtggtgtccgtgtctcccctgctgctgacctcttcccagcagaaagccgactggattcctctggccctgaccatcaaagggctgaatccgaccgccatttttctgaccaccctgtctaggacctccaaaaagagatcctggcccctgaatgaggccatcatggccgtgggcatggtgtccatcctggcctcctctctgctgaaaaatgacattccgatgaccgggcctctggtggccggcgggctgctgaccgtgtgctacgtgctgaccggcagatctgccgacctggagctggagagagccgccgacgtgcgctgggaggagcaggccgagatctccggctcctcccctatcctgtccatcaccatctctgaggacgggtccatgtccatcaaaaatgaggaggaggagcagattctgaccattctgatcagaaccggcctgctggtaatttctgggctgtttccggtgtccattcccatcaccgccgccgcctggtatctgtgggaggtgaaaaaacagagagccggcgtgctgtgggacgtgccctccccgcctcccgtggggaaagccgagctggaggacggcgcctaccgcatcaaacagaaaggcattctggggtactcccagatcggcgccggggtgtacaaagagggcacctttcacaccatgtggcacgtgaccagaggcgccgtgctgatgcataaaggcaaaaggattgagccctcttgggccgacgtgaaaaaagacctgatttcttacggcggcgggtggaaactggagggggagtggaaagagggggaggaggtgcaggtgctggccctggagcccgggaaaaatcccagagccgtgcagaccaaacctggcctgtttaaaaccaataccggcaccattggcgccgtgtccctggacttttctcccgggacctctgggtccccgattgtggacaaaaaagggaaagtggtggggctgtacgggaatggggtggtgaccaggtccggcacctacgtgtctgccattgcccagaccgagaaatccattgaggacaatcccgagattgaggacgacatctttaggaaaaaacgcctgaccattatggacctgcatcccggggccggcaaaaccaaacgctacctgcccgccattgtgagagaggccatcaaaagaggcctgaggaccctgattctggcccccaccagggtggtggccgccgagatggaggaggccctgcgcgggctgcccatccgctaccagacccccgccattagggccgagcataccggcagggagattgtggacctgatgtgccacgccacctttaccatgaggctgctgtcccccattcgcgtgccgaattacaatctgatcattatggacgaggcccactttaccgaccctgcctctatcgccgccagaggctacatctccaccagagtggagatgggggaggccgccgggatctttatgaccgccaccccccctggctccagagacccctttcctcagtccaatgcccccatcatggacgaggagagggagattcctgagaggtcttggaactctggccatgagtgggtgaccgactttaaaggcaaaaccgtgtggtttgtgccgtctatcaaagccgggaatgacattgccgcctgcctgcgcaaaaatggcaaaaaagtgattcagctgtcccgcaaaacctttgactccgagtacatcaaaacccgcaccaatgactgggactttgtggtgaccaccgacatctccgagatgggggccaatttcaaagccgagcgcgtgatcgaccccagaaggtgcatgaaacccgtgatcctgaccgacggcgaggagagagtgatcctggccgggcccatgcctgtgacccattcttctgccgcccagagaagggggcgcgtggggagaaatccgaaaaatgagtctgaccagtacatctacatgggcgagcctctggagaatgacgaggactgcgcccattggaaagaggccaaaatgctgctggacaatatcaatacccccgagggcattatcccctctatgtttgagccggagagagagaaagtggacgccattgacggcgagtacagactgcgcggcgaggccagaaaaacctttgtggacctgatgagacgcggcgacctgcccgtgtggctggcctacagggtggccgccgaggggatcaattacgccgacagacgctggtgctttgacggggtgaaaaataatcagatcctggaggagaatgtggaggtggagatttggaccaaagagggggagaggaaaaaactgaaacctaggtggctggacgccaggatttactccgaccccctggccctgaaagagtttaaagagtttgccgccggccgcaaatccctgaccctgaatctgatcaccgagatggggagactgcccacctttatgacccagaaagccagaaatgccctggacaatctggccgtgctgcacaccgccgaggccggcgggagggcctacaatcatgccctgtccgagctgcccgagaccctggagaccctgctgctgctgaccctgctggccaccgtgaccggggggatttttctgtttctgatgtctggcaaagggatcggcaaaatgaccctggggatgtgctgcattatcaccgcctccatcctgctgtggtacgcccagatccagccccactggatcgccgcctctattattctggagtttttcctgattgtgctgctgatcccggagcctgagaaacagagaaccccgcaggacaatcagctgacctacgtggtaatcgccatcctgaccgtggtggccgccaccatggccaatgagatggggtttctggagaaaaccaaaaaagactttgggctgggctcctctaccacccagcagcatgagtctaatatcctggacattgacctgagacccgcctctgcctggaccctgtacgccgtggccaccacctttatcacccctatgctgcgccattctatcgagaactcttccgtgaatgtatccctgaccgccatcgccaatcaggccaccgtgctgatggggctgggcaaaggctggcctctgtccaaaatggacatcggcgtgccgctgctggccatcggctgctactcccaggtgaatccgattaccctgaccgccgccctgctgtccctggtggcccactacgccatcattggcccggggctgcaggccaaagccaccagagaggcccagaaaagggccgccgccggcattatgaaaaatcccaccgtggacggcattaccgtgattgacctggaccctattccctacgaccccaaatttgagaaacagctggggcaggtaatgctgctggtgctgtgcgtgacccaggtgctgatgatgaggaccacctgggccctgtgcgaggccctgaccctggccaccgggccgatctctaccctgtgggaggggaatcccgggagattttggaataccaccatcgccgtgtctatggccaatatctttaggggctcttacctggccggggccggcctgctgttttccattatgaaaaataccgccaataccaggagagggaccgggaataccggggagaccctgggcgagaaatggaaaaatagactgaatgccctgggcaaatccgagtttcagatctacaaaaaatccggcatccaggaggtggacagaaccctggccaaagaggggatcaaaagaggggagaccgaccatcacgccgtgtctaggggctctgccaaactgcgctggtttgtggagaggaatctggtgaccccggaggggaaagtggtggacctgggctgcgggaggggcgggtggtcttactactgcggcggcctgaaaaatgtgaaagaggtgaaaggcctgaccaaagggggcccggggcatgaggagcccattccgatgtctacctacgggtggaatctggtgcgcctgcagtccggcgtggacgtgtttttcacccccccggagaaatgcgacaccctgctgtgcgacatcggcgagtcttccccgaatcctaccgtggaggccggccgcaccctgcgcgtgctgaatctggtggagaattggctgaataataatacccagttttgcatcaaagtgctgaatccgtacatgccctctgtgatcgagaaaatggaggccctgcagcgcaaatacggcggggccctggtgagaaatcctctgtctcgcaattctacccacgagatgtactgggtgtctaatgcctccgggaatatcgtgtcttccgtgaatatgatctccaggatgctgatcaataggtttaccatgaggcataaaaaagccacctacgagccggacgtggacctggggtctgggacccgcaatattggcatcgagtccgagacccccaatctggacattatcgggaaaaggatcgagaaaatcaaacaggagcacgagacctcttggcactacgaccaggaccatccgtacaaaacctgggcctaccacgggtcctacgagaccaaacagaccggctccgcctcttctatggtgaatggggtggtgagactgctgaccaaaccctgggacgtgatccctatggtgacccagatggccatgaccgacaccaccccgtttggccagcagcgcgtgtttaaagagaaagtggacaccagaacccaggagccgaaagaggggaccaaaaaactgatgaaaatcaccgccgagtggctgtggaaagagctgggcaaaaagaaaacccccaggatgtgcacccgcgaggagtttaccagaaaagtgagatctaatgccgccctgggggccatctttaccgacgagaataagtggaaatccgccagggaggccgtggaggactccggcttttgggagctggtggacaaagagagaaatctgcatctggagggcaaatgcgagacctgcgtgtacaatatgatgggcaaaagagagaaaaaactgggcgagtttgggaaagccaaaggctctcgcgccatctggtacatgtggctgggcgccagatttctggagtttgaggccctgggctttctgaatgaggaccactggttttcccgcgagaactccctgtccggcgtggagggcgaggggctgcataaactgggctacatcctgagggacgtgtctaaaaaagaggggggggccatgtacgccgacgacaccgccgggtgggacacccgcattaccctggaggacctgaaaaatgaggagatggtgaccaatcacatggagggggagcataaaaaactggccgaggccatctttaaactgacctaccagaataaggtggtgagggtgcagagaccgaccccgagaggcaccgtgatggacattatttctcgccgcgaccagcgcgggtccgggcaggtggtgacctacggcctgaatacctttaccaatatggaggcccagctgattagacagatggagggcgagggcgtgtttaaatccattcagcagctgaccgccaccgaggagattgccgtgaaaaattggctggtgagggtggggcgcgagaggctgtccagaatggccatttctggggacgactgcgtggtgaaacccctggacgacaggtttgcctctgccctgaccgccctgaatgacatggggaaagtgagaaaagacatccagcagtgggagccttccaggggctggaatgactggacccaggtgcccttttgctctcaccactttcacgagctgattatgaaagacgggagagtgctggtggtgccttgcagaaatcaggacgagctgattggccgcgccaggatctcccagggcgccggctggtccctgcgcgagaccgcctgcctggggaaatcttacgcccagatgtggtctctgatgtactttcacaggagagacctgaggctggccgccaatgccatttgctctgccgtgccctctcactgggtgcccacctctcgcaccacctggtctatccatgccacccatgagtggatgaccaccgaggacatgctgaccgtgtggaatagggtgtggattcaggagaatccctggatggaggacaaaacccccgtggagtcttgggaggagattccctacctggggaaaagagaggaccagtggtgcggctccctgatcggcctgacctccagggccacctgggccaaaaatatccagaccgccatcaatcaggtgaggtctctgatcggcaatgaggagtacaccgactacatgccgtctatgaagagatttcgcagagaggaggaggaagccggagtgctgtggtga

SEQ ID NO: 4 - DNA, 546 bp, Artificial Sequence
  nucleic acid sequence partial eF1 alpha promoter
aaggatctgcgatcgctccggtgcccgtcagtgggcagagcgcacatcgcccacagtccccgagaagttggggggaggggtcggcaattgaacgggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacagctgaagcttcgaggggctcgcatctctccttcacgcgcccgccgccctacctgaggccgccatccacgccggttgagtcgcgttctgccgcctcccgcctgtggtgcctcctgaactgcgtccgccgtctaggtaagtttaaagctcaggtcgagaccgggcctttgtccggcgctcccttggagcctacctagactcagccggctctccacgctttgcctgaccctgcttgctcaactctacgtctttgtttcgttttctgttctgcgccgttacagatccaagctgtgaccggcgcctac

SEQ ID NO: 5 - DNA, 1183 bp, Artificial Sequence
  nucleic acid sequence full length eEf1 alpha promoter
gtgcccgtcagtgggcagagcgcacatcgcccacagtccccgagaagttggggggaggggtcggcaattgaaccggtgcctagagaaggtggcgcggggtaaactgggaaagtgatgtcgtgtactggctccgcctttttcccgagggtgggggagaaccgtatataagtgcagtagtcgccgtgaacgttctttttcgcaacgggtttgccgccagaacacaggtaagtgccgtgtgtggttcccgcgggcctggcctctttacgggttatggcccttgcgtgccttgaattacttccacgcccctggctgcagtacgtgattcttgatcccgagcttcgggttggaagtgggtgggagagttcgaggccttgcgcttaaggagccccttcgcctcgtgcttgagttgaggcctggcctgggcgctggggccgccgcgtgcgaatctggtggcaccttcgcgcctgtctcgctgctttcgataagtctctagccatttaaaatttttgatgacctgctgcgacgctttttttctggcaagatagtcttgtaaatgcgggccaagatctgcacactggtatttcggtttttggggccgcgggcggcgacggggcccgtgcgtcccagcgcacatgttcggcgaggcggggcctgcgagcgcggccaccgagaatcggacgggggtagtctcaagctggccggcctgctctggtgcctggcctcgcgccgccgtgtatcgccccgccctgggcggcaaggctggcccggtcggcaccagttgcgtgagcggaaagatggccgcttcccggccctgctgcagggagctcaaaatggaggacgcggcgctcgggagagcgggcgggtgagtcacccacacaaaggaaaagggcctttccgtcctcagccgtcgcttcatgtgactccacggagtaccgggcgccgtccaggcacctcgattagttctcgagcttttggagtacgtcgtctttaggttggggggaggggttttatgcgatggagtttccccacactgagtgggtggagactgaagttaggccagcttggcacttgatgtaattctccttggaatttgccctttttgagtttggatcttggttcattctcaagcctcagacagtggttcaaagtttttttcttccatttcaggtgtcgtgagaattag

SEQ ID NO: 6 - DNA, 42 bp, Artificial Sequence
  nucleic acid sequence for primer D2-C-opt-T2A- Xma1-For
gtcgaggagaatcccggccctatgaacaaccagcggaagaag

SEQ ID NO: 7 - DNA, 31 bp, Artificial Sequence
  nucleic acid sequence for primer D2-E-opti- Ecor1-T2A-Rev
tccctcgacgaattctcaagcctgaaccatc

SEQ ID NO: 8 - DNA, 45 bp, Artificial Sequence
  nucleic acid sequence for primer D2-NS1-Ecor1- For
ctagagctagcgaattcgccatggcacctcactgtctgtgtcatt

SEQ ID NO: 9 - DNA, 41 bp, Artificial Sequence
  nucleic acid sequence for primer D2-NS5-BamH1- Rev
acagtcggcggccgcggatccctaccacaagactcctgcct

SEQ ID NO: 10 - DNA, 29 bp, Artificial Sequence
  nucleic acid sequence for primer pCFP-coilin For
aaaaacctaggatggtgagcaagggcgag

SEQ ID NO: 11 - DNA, 31 bp, Artificial Sequence
  nucleic acid sequence for primer pCFP-coilin Rev
tttttatgcatcttgtacagctcgtccatgc

SEQ ID NO: 12 - DNA, 31 bp, Artificial Sequence
  nucleic acid sequence for primer pCDH-EF1?-MCS- BGH-PGK-T2A-Puro For
aaaaaatgcatgagggcagaggaagtcttct

SEQ ID NO: 13 - DNA, 29 bp, Artificial Sequence
  nucleic acid sequence for primer pCDH-EF1?-MCS- BGH-PGK-T2A-Puro Rev
tttttcctaggcggtctctgctgcctcac

SEQ ID NO: 14 - DNA, 17 bp, Artificial Sequence
  nucleic acid sequence for primer human codon optimisied E gene For
accaggtgttcggcgcc

SEQ ID NO: 15 - DNA, 21 bp, Artificial Sequence
  nucleic acid sequence for primer human codon optimisied E gene Rev
ttcaagcctgaaccatcacgc

SEQ ID NO: 16 - DNA, 21 bp, Artificial Sequence
  nucleic acid sequence for primer monkey codon optimisied NS1 gene For
gagacctcagcctaccgagct

SEQ ID NO: 17 - DNA, 20 bp, Artificial Sequence
  nucleic acid sequence for primer monkey codon optimisied NS1 gene Rev
ttggagtcgcaagacacgtc

SEQ ID NO: 18 - DNA, 20 bp, Artificial Sequence
  nucleic acid sequence for NS5 gene For
gcctgatgtacttccacaga

SEQ ID NO: 19 - DNA, 21 bp, Artificial Sequence
  nucleic acid sequence for NS5 gene Rev
attgcctattagggatctaac

SEQ ID NO: 20 - DNA, 21 bp, Artificial Sequence
  nucleic acid sequence for primer monkey codon optimised NS5 gen For
tggtctatccatgccacccat

SEQ ID NO: 21 - DNA, 21 bp, Artificial Sequence
  nucleic acid sequence for primer monkey codon optimised NS5 gen Rev
atgtagtcggtgtactcctca

SEQ ID NO: 22 - DNA, 20 bp, Artificial Sequence
  nucleic acid sequence for primer quantification DENV DI RNA For
gagagaaaccgcgtgtcgac

SEQ ID NO: 23 - DNA, 20 bp, Artificial Sequence
  nucleic acid sequence for primer quantification DENV DI RNA Rev
agaacctgttgattcaacag

SEQ ID NO: 24 - DNA, 20 bp, Artificial Sequence
  nucleic acid sequence for primer GAPDH For
gcaaattccatggcaccgtc

SEQ ID NO: 25 - DNA, 19 bp, Artificial Sequence
  nucleic acid sequence for primer GAPDH Rev
tcgccccacttgattttgg

SEQ ID NO: 26 - DNA, 443 bp, Artificial Sequence
  nucleic acid sequence DENV-1 DI-RNA 443
agttgttagtctacgtggaccgacaagaacagtttcgaatcggaagcttgcttaacgtagttctaacagttttttattagagagcagatctctgatgaacaaccaacgaaaaaagacggctcgaccgtctttcaatatgctggaacgcgcgagaaaccgcgtgtcaactgtttcacagttggcgaagagattctcaaaaggattgctcttaggccaaggacccatgaaattggtgatggctttcatagcattcctaagatttctagccatacccccaactgtaccctggtggtaaggactagaggttagaggagaccccccgcataacaatgaacagcatattgacgctgggagagaccagagatcctgctgtctctacagcatcattcctggcacagaacgccagaaaatggaatggtgctgttgaatcaacaggttctatc

SEQ ID NO: 27 - DNA, 290 bp, Artificial Sequence
  nucleic acid sequence DENV-2 DI-RNA 290
agttgttagtctacgtggaccgacaaagacagattctttgagggagctaagctcaacgtagttctaacagttttttaattagagagcagatctctgatgaataaccaacggaaaaaggcgaaaaacacgcctttcaatatgctgaaacgcgagagaaaccgcgtgtcgactgtgaaacaaaaaacagcatattgacgctgggaaagaccagagatcctgctgtctcctcagcatcattccaggcacagaacgccagaaaatggaatggtgctgttgaatcaacaggttct

(19) United States
(12) Patent Application Publication
(10) Pub. No.: US 2023/0040417 A1
(43) Pub. Date: Feb. 9, 2023

(54) DEFECTIVE INTERFERING PARTICLES

(71) Applicant: The Council of the Queensland Institute of Medical Research, Herston, Queensland (AU)

(72) Inventors: David Allen Harrich, Brisbane, Queensland (AU); Dongsheng LI, Brisbane, Queensland (AU); Min-Hsuan Lin, Brisbane, Queensland (AU)

(73) Assignee: The Council of the Queensland Institute of Medical Research, Herston, Queensland (AU)

(21) Appl. No.: 17/782,544

(22) PCT Filed: Dec. 3, 2020
(86) PCT No.: PCT/AU2020/051322

§ 371 Date: Jun. 3, 2022

(30) Foreign Application Priority Data
Dec. 3, 2019 (AU) 2019904577

(51) Int. Cl. C12N 15/86; C12N 7/00; A61K 35/76; A61P 31/14

(52) U.S. Cl. CPC C12N 15/86; C12N 7/00; A61K 35/76; A61P 31/14; C12N 2770/24111

(57) ABSTRACT

The present disclosure relates to the production of transmissible vims defective interfering particles (DIPs), particularly those of dengue virus as well as methods of their production. The DIPs have particular utility as immunogenic compositions and vaccines.

### RELATED APPLICATIONS

This application claims priority to Australian Provisional Application No. 2019904577, entitled “Defective Interfering Particles” filed 3 Dec. 2019, the contents of which are incorporated herein by reference in their entireity.

### INCORPORATION BY REFERENCE

All documents cited or referenced herein, and all documents cited or referenced in herein cited documents, together with any manufacturer's instructions, descriptions, product specifications, and product sheets for any products mentioned herein or in any document incorporated by reference herein, are hereby incorporated herein by reference in their entirety.

The entire content of the electronic submission of the sequence listing is incorporated by reference in its entirety for all purposes.

### FIELD OF THE DISCLOSURE

The present disclosure relates to the production of transmissible virus defective interfering particles (DIPs), particularly those of dengue virus as well as methods of their production. The DIPs have particular utility as immunogenic compositions, vaccines and reducing viral transmission.

### BACKGROUND OF THE DISCLOSURE

Flaviviridae viruses are important arthropod born (e.g. mosquito and tick) viral diseases that infect a range of hosts including humans, mammals, avians and livestock resulting in disease.

Dengue virus (DENV) is a mosquito-borne Flaviviridae that cases disease in humans. DENV inflicts 390 million people annually in more than 100 countries. DENV is a Flavivirus (in the same family as Zika virus (ZIKV) and West Nile Virus (WNV)) with a small positive strand RNA genome. There are four serotypes, DENV-1 to -4 that are all transmitted by mosquitoes and cause ˜100 million clinical infections annually, about ½ million hospitalisations and 25,000 deaths. Severe DENV disease may occur after a patient clears an infection by one serotype and is subsequently infected by a different DENV serotype. Currently, antiviral drugs are not available for clinical use. The first licensed dengue vaccine is available but its use and effectiveness are limited. Severe disease exacerbated by antibody dependent enhancement is currently not treatable.

A Defective Interfering Particle (DIP) refers to a defective virion that was first reported more than 70 years ago for influenza A virus. DIPs have been found in laboratory cultures, in viruses infected animals and patients. DIPs are defined as virus-like particles that: i.) contain a normal or partial set of viral proteins; ii.) contain a partial parental viral genome, which is referred to as a defective interfering (DI) genome- or defective interfering RNA (DI RNA); iii.) are unable to reproduce independently. They can use the replication machinery produced by the parental virus (also referred to as helper virus) for replication.

In vitro experiments showed that DIPs inhibit virus replication in a dose-dependent manner and interference by natural DIPs has been inferred in vivo by loss in pathogenicity and changes in recovery rates from viral infection in animal hosts. Exactly how DIPs reduce wild type virus replication from which they are derived has not been fully elucidated. DIPs parasitise cellular and viral resources required by the wild-type virus for replication (Barrett and Dimmock, 1986) and may stimulate cellular innate antiviral responses. For these reasons, DIPs have been evaluated as therapeutic agents for a range of RNA viruses. However, their clinical use has been hampered by DIP preparations that are contaminated by infectious parental virus that is impractical to remove.

### SUMMARY OF THE DISCLOSURE

The present inventors have developed virus interfering particles (DIPs), more particularly Dengue virus interfering particles and methods for their production in vitro. By expressing the viral structural and non-structural proteins on separate vectors, the chances of recombination events to produce infectious virus are reduced. Moreover, the use of self-inactivating vectors provides a further mechanism by which recombination events to produce infectious virus are reduced. Moreover, the DIPS can be produced that are free of standard helper infectious virus.

In one aspect, the disclosure provides a cell line for producing virus defective interfering particles (DIPs), comprising:

(i) a first vector for expression of the non-structural proteins of a virus of the Flaviviridae family;

(ii) a second vector for expression of the structural proteins of a virus of the Flaviviridae family (i); and

wherein, upon the introduction of a third vector for the expression of a Flaviviridae defective interfering genomic sequence the cell produces DIPs.

In one example, the virus of the Flaviviridae family of (i) and (ii) are the same virus.

In one example, the virus of the Flaviviridae family of (i) and (ii) are not the same virus.

In one example, the virus of the Flaviviridae family of (i) and/or (ii) is selected from a: Flavivirus, Hepacivirus, Pegivirus, Pestivirus, and Jingmenvirus. In one example, the Flaviviridae is a Flavivirus, In one example, the Flavivirus is selected from the group consisting of: Dengue virus (DENV), West Nile virus (WNV), Yaounde virus, Yellow fever virus (YFV), Zika virus (ZIKA), Apoi virus, Aroa virus, Bagaza virus, Banzi virus, Bouboui virus, Bukalasa bat virus, Cacipacore virus, Carey Island virus, Cowbone Ridge virus, Dakar bat virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Penh bat virus, Powassan virus, Rio Bravo virus, Royal Farm virus, Saboya virus, Sal Vieja virus, San Perlita virus, Saumarez Reef virus, Sepik virus, St. Louis encephalitis virus, Tembusu virus, Tick-borne encephalitis virus, Tyuleniy virus, Uganda S virus, Usutu virus, Wesselsbron virus, and Yokose virus.

In one example, the Flavivirus is selected from: DENV, ZIKA, WNV, and YFV. In one example, the Flavivirus is DENV. In one example, the DENV serotype is selected from one or more of: DENV1, DENV2, DENV3, and DENV4. In one example, the DENV serotype is DENV1. In one example, DENV1 comprises the sequence of GenBank accession no. AY726554.1. In one example, the DENV serotype is DENV2. In one example, DENV2 comprises the sequence of GenBank accession No. AF169688.1. In one example, DENV2 comprises the sequence of GenBank accession No. AF038403.1. In one example, the DENV serotype is DENV3. In one example, DENV2 comprises the sequence of GenBank accession No. FN429913.1. In one example, the DENV serotype is DENV4. In one example, DENV4 comprises the sequence of GenBank accession No. AY618990.1.

In one example, the virus of the Flaviviridae family of (i) is selected from DENV, ZIKA, WNV, and YFV.

In one example, the virus of the Flaviviridae family of (ii) is selected from DENV, ZIKA, WNV, and YFV.

In one example, the virus of the Flaviviridae family of (i) or (ii) is a DENV serotype selected from: DENV1, DENV2, DENV3, and DENV4.

In one example, the virus of the Flaviviridae family of (i) and (ii) is a DENV serotype selected from one or more of: DENV1, DENV2, DENV3, and DENV4.

In one example, the DIP is capable of only a single round of infection. For example, once the DIP infects a cell, it is able to integrate into the host cell genome but is unable to produce further virus particles without assistance from wild-type virus or without the viral structural and non-structural proteins being provided in trans.

In one example, the virus defective interfering genomic sequence is modified relative to the genomic sequence of its corresponding infectious native viral genomic sequence. In one example, the modification is an internal deletion of genomic sequence. In a further example, the virus defective interfering genomic sequence does not include the genes encoding viral structural and non-structural proteins. In another example, the virus defective interfering genomic sequence comprises about 3 to 10% of the total viral genomic sequence relative to the corresponding native virus.

In one example, the virus defective interfering genomic is expressed and packaged as RNA.

In yet another example, the defective interfering genomic sequence is expressed from a sequence selected from the group comprising or consisting of SEQ ID NO:26 or SEQ ID NO:27.

In yet another example, the defective interfering genomic sequence is expressed from a sequence selected from the group comprising or consisting of any one of SEQ ID NO:28 to SEQ ID NO:41.

In some examples, the cell line comprises:

(i) a first vector for expression of the non-structural proteins of a virus of the Flaviviridae family;

(ii) a second vector for expression of the structural proteins of a virus of the Flaviviridae family (i); and

(iii) a third vector for the expression of a Flaviviridae defective interfering genomic sequence.

In one example, the virus of Flaviviridae family of (i) and (ii) are the same virus.

In one example, the virus of Flaviviridae family of (i) and (ii) are not the same virus.

In one example, the first vector comprises Flaviviridae non-structural proteins. In another example, the non-structural proteins comprise one or more, or all of NS1, NS2A, NS2B, NS3, NS4A, NS4B and NS5 of a Flavivirus, more particularly Dengue virus.

In another example, the second vector comprises Flaviviridae structural proteins. In another example, the structural proteins comprise one or more or all of capsid (C), pre-membrane/membrane (prM), and envelope (E).

In another example, the third vector comprises a Flaviviridae defective interfering genomic sequence. In a further example, the third vector comprises a DNA sequence encoding a Flaviviridae defective interfering genomic sequence. In a further example, the sequence comprises an internal deletion of the genomic sequence. In a further example, the virus defective interfering genomic sequence does not include the genes encoding viral structural and non-structural proteins. In another example, the virus defective interfering genomic sequence comprises about 3 to 10% of the total viral genomic sequence relative to the corresponding native virus.

The first, second and third vectors may be the same of different. For example, the first, second and third vectors may be retroviral or lentiviral vectors or a combination thereof. In one example, the first and third vectors are lentivirus vectors. In another example, the second vector is a retrovirus vector.

The first vector and the second vector may be separate vectors or a contiguous vector. In another example, the structural and non-structural proteins are expressed by a single promoter.

In one example one or more of the first, second, and third vectors are self-inactivating (SIN) vectors. In a further example, the SIN vector comprises a deletion of a portion of the long terminal repeat (LTR) sequence. This deletion may be present in either the 5′ or 3′ LTR or in both the 5′ and 3′ LTRs. In a further example, the deletion is a U3, R or U5 sequence deletion of the LTR. In a further example, the deletion is a U3 sequence deletion. In a still further example, the SIN vector is the second vector.

In one example, the first, second and third vectors are integrated into the genome of the cell line.

In a further example, the introduction of the third vector into the cell may be by transfection or transduction. In a further example, the introduction of the third vector into the cell may be by direct injection.

In one example, the structural proteins and/or non-structural proteins are human and/or Old World monkey codon optimised. In some examples, one of the vectors may be human codon optimised and another African Green Monkey codon optimised. For example, if the first vector is human codon optimised, the second vector is Old World monkey codon optimised or vice versa.

In one example, the virus defective interfering genomic sequence is constitutively expressed in the cell line.

In one example, the expressed DIPs are secreted from the cell line. In one example, the expressed DIPs are continuously secreted from the cell line. In one example, DIPs are constitutively produced in the cell line. In a further example, the cell line produces DIPs in concentration range of from about 1×10⁷ to about 1×10⁸ DIP RNA copies/ml.

In one example, the defective interfering genomic sequence comprises 5′ and/or 3′ regulatory sequences. In one example, the regulatory sequences comprise the 5′untraslated region (UTR), 5′ upstream AUG region (UAR), the motif downstream of the AUG region (DAR), some intervening sequence and the 3′ end of the 3′UTR including the 3′ conserved sequence (CS) and the 3′ UAR. In another example, the defective interfering genomic sequence comprises a large internal deletion. In yet another example, the internal deletion is at least about 80%, at least about 90%, at least about 95%, or at least about 97% of the genomic sequence.

In one example, the defective interfering genomic sequence comprises about 155 nucleotides to about 1000 nucleotides. In one example, the defective interfering genomic sequence comprises about 200 nucleotides to about 800 nucleotides. In one example, the defective interfering genomic sequence comprises about 200 nucleotides to about 500 nucleotides.

In one example, the defective interfering genomic sequence is cloned as DNA or cDNA. In another example, the defective interfering genomic sequence is expressed and packaged as an RNA. In another example, the RNA is a positive strand RNA.

The Flaviviridae according to the disclosure may be selected from: Flavivirus, Hepacivirus, Pegivirus, Pestivirus, and Jingmenvirus. In one example, the Flaviviridae is a Flavivirus, In one example, the Flavivirus is selected from the group consisting of: Dengue virus (DENV), West Nile virus (WNV), Yaounde virus, Yellow fever virus (YFV), Zika virus (ZIKA), Apoi virus, Aroa virus, Bagaza virus, Banzi virus, Bouboui virus, Bukalasa bat virus, Cacipacore virus, Carey Island virus, Cowbone Ridge virus, Dakar bat virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Penh bat virus, Powassan virus, Rio Bravo virus, Royal Farm virus, Saboya virus, Sal Vieja virus, San Perlita virus, Saumarez Reef virus, Sepik virus, St. Louis encephalitis virus, Tembusu virus, Tick-borne encephalitis virus, Tyuleniy virus, Uganda S virus, Usutu virus, Wesselsbron virus, and Yokose virus.

In one example, the Flavivirus is selected from: DENV, ZIKA, WNV, and YFV. In one example, the Flavivirus is DENV. In one example, the DENV serotype is selected from one or more of: DENV1, DENV2, DENV3, and DENV4. In one example, the DENV serotype is DENV1. In one example, DENV1 comprises the sequence of GenBank accession no. AY726554.1. In one example, the DENV serotype is DENV2. In one example, DENV2 comprises the sequence of GenBank accession No. AF169688.1. In one example, DENV2 comprises the sequence of GenBank accession No. AF038403.1. In one example, the DENV serotype is DENV3. In one example, DENV2 comprises the sequence of GenBank accession No. FN429913.1. In one example, the DENV serotype is DENV4. In one example, DENV4 comprises the sequence of GenBank accession No. AY618990.1.

The cell line according to the disclosure may be a human cell or a primate cell. In one example, the cell line is selected from a Vero cell or HEK 293 cell, more particularly a HEK293T cell.

In another aspect, the disclosure provides a method for producing virus defective interfering particles (DIPs), comprising transfecting or transducing a cell line as described herein with a vector comprising a Flaviviridae defective interfering genomic sequence as described herein, wherein the cell line comprises (i) a first vector which expresses the non-structural proteins of a virus of the Flaviviridae family; and (ii) a second vector which expresses the structural proteins of the same virus according to (i); and wherein when the Flaviviridae defective interfering genomic sequence is expressed in the cell line by a third vector, the cell line produces DIPs.

In another aspect, the disclosure provides a method for producing virus defective interfering particles (DIPs), comprising expressing a Flaviviridae defective interfering genomic sequence as described herein in a cell line comprising: i) a first vector which expresses the non-structural proteins of a virus of the Flaviviridae family; and (ii) a second vector which expresses the structural proteins of the same virus according to (i); and wherein when the Flaviviridae defective interfering genomic sequence is expressed in the cell line by a third vector, the cell line produces DIPs.

In one example, the method further comprises culturing the cell line in stationary culture, stirred culture, or bioreactor. In one example, the cell line is cultured in serum free cell culture medium. In one example, the cell line is cultured in Happy Cell® Advanced Suspension Medium. In one example, the cell line is cultured at about 37° C. to about 40° C. In one example, the cell line is cultured at about 38° C. to about 39.5° C. In one example, the cell line is cultured at about 39° C.

In another aspect, the disclosure provides a cloned or recombinant virus defective interfering particle (DIP) or population of DIPs expressed by the cell line as described herein, or produced by the method as described herein.

In another aspect, the disclosure provides an isolated virus defective interfering particle (DIP) or population of DIPs expressed by the cell line as described herein, or produced by the method as described herein.

In one example, the DIP as described herein has an antiviral effect against one or more of: i) an RNA virus; ii) a single stranded RNA virus; and iii) a positive single stranded RNA virus.

In one example, the DIP as described herein has an antiviral effect against one or more subtypes of DENV selected from: DENV1, DENV2, DENV3, DENV4.

In one example, the DIP can bind to and enter (i.e. infect) a Flaviviridae host or Flaviviridae carrier cell not infected with a wild type Flaviviridae virus.

In one example, the DIP can bind, enter and replicate in a Flaviviridae host or Flaviviridae carrier cell comprising a wild type Flaviviridae virus.

In a further aspect, the disclosure provides a pharmaceutical composition comprising the DIP as described herein. In one example, the composition comprises a pharmaceutically acceptable carrier or excipient.

In yet another aspect, the disclosure provides an immunogenic composition comprising a DIP as described herein. In one example, the immunogenic composition is a vaccine.

In another aspect, the disclosure provides a method of treating or preventing a Flaviviridae disease comprising administering to a subject in need thereof the DIP as described herein, the pharmaceutical composition as described herein or the immunogenic composition as described herein.

In one example, the Flaviviridae disease is selected from one or more of: fever, rash, myalgia, haemorrhagic fever, abortion, encephalitis, neonatal encephalitis, egg-drop-syndrome, neuroparalytic disease, myocardial necrosis, hepato- and splenomegaly, congenital disease, acute dengue disease, severe dengue disease and severe dengue disease caused by antibody dependent enhancement.

In another aspect, the disclosure provides a method of reducing the load of a Flavivirus RNA in a subject comprising administering to the subject the DIP as described herein, the pharmaceutical composition as described herein or the immunogenic composition as described herein.

In another aspect, the disclosure provides a method of reducing transmission of a Flaviviridae between a Flaviviridae host and a Flaviviridae carrier comprising administering the DIP as described herein, the pharmaceutical composition as described herein or the immunogenic composition as described herein to the Flaviviridae host.

In one example, the DIP or composition comprising same is administered in one or more of the following conditions:

i) before a subject/host is infected with a Flaviviridae;

ii) if a subject/host has been in contact with an individual infected with a Flaviviridae or in contact with a Flaviviridae;

iii) after a subject/host is infected with a Flaviviridae;

iv) as a single dose;

v) in two or more doses.

In another aspect, the disclosure provides a method of reducing transmission of a Flaviviridae between a Flaviviridae host and a Flaviviridae carrier comprising administering the DIP as described herein, the pharmaceutical composition as described herein or the immunogenic composition as described herein to the Flaviviridae carrier.

In another aspect, the disclosure provides use of a DIP as described herein, in the manufacture of a medicament for treating or preventing a Flaviviridae disease in a subject.

In another aspect, the disclosure provides use of a virus defective interfering particle (DIP), as described herein in the manufacture of a medicament for reducing the load of an RNA virus in a subject.

In another aspect, the disclosure provides use of a virus defective interfering particle (DIP) as described herein in the manufacture of a medicament for reducing transmission of a Flaviviridae between a Flaviviridae host and a Flaviviridae carrier.

In another aspect, the disclosure provides a vector comprising a Dengue virus defective interfering genomic sequence encoding a Dengue virus interfering RNA sequence, wherein the vector is capable of inhibiting replication by a wild-type Dengue virus. In one example, the vector is capable of inhibiting replication of wild-type Dengue virus present in a cell or a host when the vector is introduced into the cell or host.

In another aspect, the disclosure provides a sequence encoding a Dengue virus defective interfering RNA sequence, wherein the RNA sequence is capable of inhibiting replication by a wild-type Dengue virus.

In another aspect, the disclosure provides a method of inhibiting replication by a wild-type Dengue virus in a cell or a host infected with the Dengue virus comprising administering to the cell or host a sequence encoding a Dengue virus interfering RNA sequence.

In one example, the defective interfering genomic sequence comprises about 155 nucleotides to about 1020 nucleotides. In one example, the defective interfering sequence comprises about 200 nucleotides to about 800 nucleotides. In one example, the defective interfering genomic sequence comprises about 200 nucleotides to about 500 nucleotides.

In one example, the defective interfering sequence is selected from the group consisting of:

(i) the sequence of Genbank accession number HM016528;

(SEQ ID NO: 28)
AGTTGTTAGTCTACGTGGACCGACAAAGACAGATTCTTTGAGGGAGCTAA
GCTCAACGTAGTTCCAACAGTTTTTTAATTAGAGAGCAGATCTCTGATGA
ATAACCAACGAAAAAAGGCGAGAAATACGCCTTTCAATATGCTGAAACGC
GAGAGAAACCGCGTGTCGACTGTACAACAGCTGACAAAGACAAATCGCAG
CAACAATGGGGGCCCAAGGTGAGATGAAGCTGTAGTCTCACTGGAAGGAC
TAGAGGTTAGAGGAGACCCCCCCAAAACAAAAAACAGCATATTGACGCTG
GGAAA GACCAGAGATCCTGCTGTCTCCTCAGCATCATTCCAGGCACAGA
ACGCCAGAAAATGGAATGGTGCTGTTGAATCAACAGGTTCT;

(ii) the sequence of Genbank accession number HM016527;

(SEQ ID NO: 29)
AGTTGTTAGTCTACGTGGACCGACAAAGACAGATTCTTTGAGGGAGCTAA
GCTCAACGTAGTTCCAACAGTTTTTTAATTAGAGAGCAGATCTCTGATGA
ATAACCAACGAAAAAAGGCGAGAAATACGCCTTTCAATATGCTGAAACGC
GAGAGAAACCGCGTGTCGACTGTACAATGGGGGCCCAAGGTGAGATGAAG
CTGTAGTCTCACTGGAAGGACTAGAGGTTAGAGGAGACCCCCCCAAAACA
AAAAACAGCATATTGACGCTGGGAAAGACCAGAGATCCTGCTGTCTCCTC
AGTATCATTCCAGGCACAGAACGCCAGAAAATGGAATGGTGCTGTTGAAT
CAACAGGTTT;

(iii) the sequence of Genbank accession number HM016525;

(SEQ ID NO: 30)
AGTTGTTAGT CTGTGTGGAC CGACAAGGAC AGTTCCAAAT
CGGAAGCTTG CTTAACACAGTTCTAACAGTTTGTTTTAGATAGAGAGCA
GATCTCTGGAAAAATGAACCAACGAAAAAAGGTGGCCAGACCACCTTTCA
ATATGCTGAAACGCGAGAGAAACCGCGTATCAACCCCTCAAGGGTTGGTG
AAGAGATTCTCGACTGGACTTTTTCCGGGAAAGGACCCTTACGGATGGTG
TTGGCATTCATTACGTTTTTGAGAGTTCTTTCCATCCCACCAACAGCAGG
GATTCTGAAAAGATGGGGACAGTTAAAGAAAAACAAGGCCATAAAGGTAA
GGAAAGACATTCCACAATGGGAACCATCCAAGGGATGGAAAAACTGGCAA
GAGGTTCCCTTTTGCTCCCACCACTTCCACAAGATTGACAAAACTCCAGT
TCACTCGTGGGAAGACATACCTTACCTAGGGAAAAGAGAGGATTTGTGGT
GTGGATCCTTGATTGGACTCTCTTCTAGGGCCACCTGGGCTAAAAACATC
CACACAGCCATAACCCAGGTCAGGAACCTGATCGGGAAAGAGGAGTATGT
GGATTACATGCCAGTCATGAAAGATACAGCGCTCATTTCGAGAGTGAAGG
AGTTCTGTAATCACCAACAAAAAACCCCAAAGAGGCTATTGAAGTCAGGC
CACTTGTGCCACGGCTTGAGCAAACCGTGCTGCCTGTAGCTCCGCCAATA
ACGGGAGGCGTAAAAATCCCGGGGAGGCCATGCGCCACGGAAGCTGTACG
CGTGGCATATTGGACTAGCGGTTAGAGGAGACCCCTCCCATCACCAACTA
AACGCAGCAAAAAGGGGGCCCGAAGCCAGGAGGAAGCTGTACTCCTGGTG
GAAGGACTAGAGGTTAGAGGAGACCCCCCTCAACACAAAAAACAGCATAT
TGACGCTGGGAAAGACCAGAAGATCCTGC TGTCCTCTGC
AACATCAATC AGGCCACAGA CGCCGCGAGA ATGG;

(iv) the sequence of Genbank accession number HM016524;

(SEQ ID NO: 31)
AGTTGTTAGTCTGTGTGGACCGACAAGGACAGTTCCAAATCGGAAGCTTG
CTTAACACAGTTCTAACAGTTTGTTTTAGATAGAGAGCAGATCTCTGGAA
AAATGAACCAACGAAAAAAGGTGGCCAGACCACCTTTCAATATGCTGAAA
CGCGAGAGAAACCGCGTATCAACCCCTCAAGGGTTGGTGAAGAGATTCTC
GACTGGACTTTTTTCCGGGAAAGGACCCTTACGGATGGTGTTGGCATTCA
TTACGTTTTTGAGAGTTCTTTCCATCCCACCAACAGCAGGGATTTGTGGT
GTGGATCCTTGATTGGACTCTCTTCTGGGGCCACCTGGGCTAAAAACATC
CACACAGCCATAACCCAGGTCAGGAACCTGATCGGGAAAGAGGAGTATGT
GGATTACATGCCAGTCATGAAAAGATACAGCGCTCATTTCGAGAGTGAAG
GAGTTCTGTAATCACCAACAAAAAACCCCAAAGAGGCTATTGAAGTCAGG
CCACTTGTGCCACGGCTTGAGCAAACCGTGCTGCCTGTAGCTCCGCCAAT
AACGGGAGGCGTAAAAATCCCGGGGAGGCCATGCGCCACGGAAGCTGTAC
GCGTGACATATTGACTAGCGGTTAGAGGAGACCCCTCCCATCACCAACTA
AACGCAGCAAAAAGGGGGCCCGAAGCCAGGAGGAAGCTGTACTCCTGTGA
AGGACTAGAGGTTAGAGGAGACCCCCCAACACAAAACAGCATATTGACGC
TGGGAAAGACCAGAGATCCTGCTGTCTCTGCAACATCAATCCAGGCACAG
AGCGCCGCGA GATGATTGTGTTGTGATCCA CAGGTTCT;

(v) the sequence of Genbank accession number HM016523;

(SEQ ID NO: 32)
AGTTGTTAGTCTGTGTGGACCGACAAGGACAGTTCCAAATCGGAAGCTTG
CTTAACACAGTTCTGACAGTTTGTTTTAGATAGAGAGCAGATCTCTGGAA
AAATGAACCAACGAAAAAAGGTGCCAGACCACCTTTCAATATGCTGAAAC
GCGAGAGAAACCGCGTATCAACCCCTCAAGGGTTGGTGAAGAGATTCTCG
ACTGGACTTTTTTCCGGGAAAGGACCCTTACGGATGGTGTTGGCATTCAT
TACGTTTTTGGGAGTTCTTTCCATCCCACCAACAGCAGGGATTCTGAAAA
GATGGGGACAGTTAAAGAAAAACAAGGCCATAAAGAGGCTATTGAAGTCA
GGCCACTTGTGCCACGGCTTGAGCAAACCGTGCTGCCTGTAGCTCCGCCA
ATAACGGGAGGCGTAAAAATCCCGGGGAGGCCATGCGCCACGGAAGCTGT
ACGCGTGGCATATTGGACTAGCGGTTAGAGGAGACCCCTCCCATCACCAA
CTAAACGCAGCAAAAAGGGGGCCCGAAGCCAGGAGGAAGCTGTACTCCTG
GTGGAAGGACTAGAGGTTAGAGGAGACCCCCCCAACACAAAAACAGCATA
TTGACGCTGGGAAAGACCAGAGATCCTGCTGTCTCTGCAACATCAATCCA
GGCACAGAGCGCCGCGAGATGGATTGGTGTTGTTGATCCAACAGGTTCT;

(vi) the sequence of Genbank accession number HM016522;

(SEQ ID NO: 33)
AGTTGTTAGTCTGTGTGGACCGACAAGGACAGTTCCAAATCGGAAGCTT
GCTTAACACAGTTCTGACAGTTTGTTTTAGATAGAGAGCAGATCTCTGG
AAAAATGAACCAACGAAAAAAGGTGGCCAGACCACCTTTCATATGCTGA
AACGCGAGAGAAACCGCGTATCAACCCCTCAAGGGTTGGTGAAGAGATT
CTCGACTGGACTTTTTTCCGGGAAAGGACCCTTACGGATGGTGTTGGCA
TTCATTACGTTTTTGGGAGTTCTTTCCATCCCACCAACAGCAGGGATTC
TGAAAAGATGGGGACAGTTAAAGAAAAACAAGGCCATAAAGAGGCTATT
GAAGTCAGGCCACTTGTGCCACGGCTTGAGCAAACCGTGCTGCCTGTAG
CTCCGCCAATAACGGGAGGCGTAAAAATCCCGGGGAGGCCATGCGCCAC
GGAAGCTGTACGCGTGGCATATTGGACTAGCGGTTAGAGGAGACCCCTC
CCATCACCAACTAAACGCAGCAAAAAGGGGGCCCGAAGCCAGGAGGAAG
CTGTACTCCTGGTGGAAGGACTAGAGGTTAGAGGAGACCCCCCCAACAC
AAAAACAGCATATTGACGCTGGGAAAGACCAGAGATCCTGCTGTCTCTG
CAACATCAATCCAGGCACAGAGCGCCGCGAGATGGATTGGTGTTGTTGA
TCCAACAGGTTCT;

(vii) the sequence of Genbank accession number HM016521;

(SEQ ID NO: 34)
AGTTGTTAGTCTGTGTGGACCGACAAGGACAGTTCCAAATCGGAAGCTTG
CTTAACACAGTTCTAACAGTTTGTTTTAGATAGAGAGCAGATCTCTGGAA
AAATGAACCAACGAAAAAAGGTGGCCAGACCACCTTTCAATATGCTGAAA
CGCGAGAGAAACCGCGTATCAACCCCTCAAGGGTTGGTGAAGAGATTGTC
GACTGGACTTTTTTCCGGGAAAGGACCCTTACGGATGGTGTTGGCATTCA
TTACGTTTTTGAGAGTTCTTTCCATCCCACCAACAGCAGGGATTCTGAAA
AGATGGGGACAGTTAAAGAAAAACAAGGCCATAAAGATACTAACTGGATT
CAGGAAGGAGATAGGCCGCATGCTGAACATCTTGAATGGAAGGAAAAGGT
CAACACAAAAACAGCATATTGACGCTGGGAAAGACCAGAGATCCTGCTGT
CTCTGCAACATCAATCCAGGCACAGAGCGCCGCGAGATGGATTGGTGTTG
TTGATCCAACAGGTTCT;

(viii) the sequence of Genbank accession number HM016520;

(SEQ ID NO: 35)
AGTTGTTAGTCTGTGTGGACCGACAAGGACAGTTCCAAATCGGAAGCTTG
CTTAACACAGTTCTAACAGTTTGTTTTAGATAGAGAGCAGATCTCTGGAA
AAATGAACCAACGAAAAAAGGTGGCCAGACCACCTTTCAATATGCTGAAA
CGCGAGAGAAACCGCGTATCAACCCCTCAAGGGTTGGTGAAGAGATTCTC
GACTGGACTTTTTTCCGGGAAAGGACCCTTACGGATGGTGTTGGCATTCA
TTACGTTTTTGAGAGTTCTTTCCATCCCACCAACAGCAGGGATTCTGAAA
AGATGGGGACAGTTAAAGAAAAACAAGGCCATAAAGATACTAACTGGATT
CAGGAAGGAGATAGGCCGCATGCTGAACATCTTGAATGGAAGGAAAAGGT
CAACACAAAAACAGCATATTGACGCTGGGAAAGACCAGAGATCCTGCTGT
CTCTGCAACATCAATCCAGGCACAGAGCGCCGCGAGATGGATTGGTGTTG
TTGATCCAACAGGTTCT;

(ix) the sequence of Genbank accession number HM016519;

(SEQ ID NO: 36)
AGTTGTTAGTCTACGTGGACCGACAAGAACAGTTTCGACTCGGAAGCTTG
CTTAACGTATGCTGACAGTTTTTTATTAGAGAGCAGATTTCTGATGAACA
ACCAACGAAAAAAGACGGGAAAACCGTCTATCAATATGCTGAAACGCGTG
AGAAACCGTGTGTCAACTGGATCACAGTTGGCGAAGAGTTAGAGGAGACC
CCTCCCATGACACAACGCAGCAGCGGGGCCCGAGCACTGAGGGAAGCTGT
ACCTCCTTGCAAAGGACTAGAGGTTAGAGGAGACCCCCCGCAAATAAAAA
CAGCATATTGACGCTGGGAGAGACCAGAGATCCTGCTGTCTCCTCAGCAT
CATTCCAGGCACAGAACGCCAGAAAATGGAATGGTGCTGTTGAATCAACA
GGTTCT;

(x) the sequence of Genbank accession number HM016518;

(SEQ ID NO: 37)
AGTTGTTAGTCTACGTGGACCGACAAGAACAGTTTCGACTCGGAAGCTTG
CTTAACGTAGTGCTGACAGTTTTTTATTAGAGAGCAGATTTCTGATGAAC
AACCAACGAAAAAAGACGGGAAAACCGTCTATCAATATGCTGAAACGCGT
GAGAAACCGTGTGTCAACTGGATCACAGTTGGCGAAGAGTTAGAGGAGAC
CCCTCCCATGACACAACGCAGCAGCGGGGCCCGAGCACTGAGGGAAGCTG
TACCTCCTTGCAAAGGACTAGAGGTTAGAGGAGACCCCCCGCAAATAAAA
ACAGCATATTGACGCTGGGAGAGACCAGAGATCCTGCTGTCTCCTCAGCA
TCATTCCAGGCACAGAACGCCAGAAAATGGAATGGTGCTGTTGAATCAAC
AGGTTCT;

(xi) the sequence of Genbank accession number HM016515;

(SEQ ID NO: 38)
AGTAGTTAGTCTACGTGGACCGACAAAGACAGATTCTTTGAGGGAGCTAA
GCTCAACGTAGTTCTAACAGTTTTTTAATTAGAGAGCAGATCTCTGATGA
ATAACCAACGGAAAAAGGCGAAAAACACGCCTTTCAATATGCTGAAACGC
GAGAGAAACCGCGTGTCGACTGTGCAACAGCTGACAAAGAGATTCTCACT
TGGAATCGCAGCAACAATGGGGGCCCAAGGCGAGATGAAGCTGTAGTCTC
GCTGGAAGGACTAGAGGTTAGAGGAGACCCCCCCGAAACAAAAAACAGCA
TATTGACGCTGGGAAAGACCAGAGATCCTGCTGTCTCCTCAGCGTCATTC
CAGGCACAGAACGCCAGAAAATGGAATGGTGCTGTTGAATCAACAGGTTC
T;

(xii) the sequence of Genbank accession number HM016514;

(SEQ ID NO: 39)
AGTAGTTAGTCTACGTGGACCGACAAAGACAGATTCTTTGAGGGAGCTAA
GCTCAACGTAGTTCTAACAGTTTTTTAATTAGAGAGCAGATCTCTGATGA
ATAACCAACGGAAAAAGGCGAAAAACACGCCTTTCAATATGCTGAAACGC
GAGAGAAACCGCGTGTCGACTGTGCAACAGCTGACAAAGAGATTCTCACT
TGGAATCGCAGCAACAATGGGGGCCCAAGGCGAGATGAAGCTGTAGTCTC
GCTGGAAGGACTAGAGGTTAGAGGAGACCCCCCCGAAACAAAAAACAGCA
TATTGACGCTGGGAAAGACCAGAGATCCTGCTGTCTCCTCAGCGTCATTC
CAGGCACAGAACGCCAGAAAATGGAATGGTGCTGTTGAATCAACAGGTTC
T;

(xiii) the sequence of Genbank accession number HM016513;

(SEQ ID NO: 40)
AGTTGTTAGTCTACGTGGACCGACAAGAACAGTTTCGAATCGGAAGCTTG
CTTAACGTAGTTCTAACAGTTTTTTATTAGAGAGCAGATCTCTGATGAAC
AACCAACGAAAAAAGACGGCTCGACCGTCTTTCAATATGCTGAAACGCGC
GAGAAACCGCGTGTCAACTGTTTCACAATTGGCGAAGAGATTCTCAAAAG
GATTGCTCTCAGGCCAAGGACCCATGAAATTGGTGATGGCCTTCATAGCA
TTCCTAACAATAAACAGCATATTGACGCTGGGAGAGGCCGGAGATCCTGC
TGTCTCTACAGCATCATTCCAGGCACAGAACGCCAGAAAATGGAATGGTG
CTGTTGAATCAACAGGTTCA;

(xiv) the sequence of Genbank accession number HM016512;

(SEQ ID NO: 41)
AGTTGTTAGTCTACGTGGACCGACAAGAACAGTTTCGAATCGGAAGCTTG
CTTAACGTAGTTCTAACAGTTTTTTATTAGAGAGCAGATCTCTGATGAAC
AACCAACGAAAAAAGACGGCTCGACCGTCTTTCAATATGCTGAAACGCGC
GAGAAACCGCGTGTCAACTGTTTCACAATTGGCGAAGAGATTCTCAAAAG
GATTGCTCTCAGGCCAAGGACCCATGAAATTGGTGATGGCCTTCATAGCA
TTCCTAACAATAAACAGCATATTGACGCTGGGAGAGGCCGGAGATCCTGC
TGTCTCTACAGCATCATTCCAGGCACAGAACGCCAGAAAATGGAATGGTG
CTGTTGAATCAACAGGTTCA.

In a further example, the defective interfering sequence is selected from:

(i) DENV-1 DI-RNA 443 comprising or consisting of the sequence

AGTTGTTAGTCTACGTGGACCGACAAGAACAGTTTCGAATCGGAAGCTTG
CTTAACGTAGTTCTAACAGTTTTTTATTAGAGAGCAGATCTCTGATGAAC
AACCAACGAAAAAAGACGGCTCGACCGTCTTTCAATATGCTGGAACGCGC
GAGAAACCGCGTGTCAACTGTTTCACAGTTGGCGAAGAGATTCTCAAAAG
GATTGCTCTTAGGCCAAGGACCCATGAAATTGGTGATGGCTTTCATAGCA
TTCCTAAGATTTCTAGCCATACCCCCAACTGTACCCTGGTGGTAAGGACT
AGAGGTTAGAGGAGACCCCCCGCATAACAATGAACAGCATATTGACGCTG
GGAGAGACCAGAGATCCTGCTGTCTCTACAGCATCATTCCTGGCACAGAA
CGCCAGAAAATGGAATGGTGCTGTTGAATCAACAGGTTCTATC;

and

(ii) DENV-2 DI-RNA 290 comprising or consisting of the sequence

AGTTGTTAGTCTACGTGGACCGACAAAGACAGATTCTTTGAGGGAGCTAA
GCTCAACGTAGTTCTAACAGTTTTTTAATTAGAGAGCAGATCTCTGATGA
ATAACCAACGGAAAAAGGCGAAAAACACGCCTTTCAATATGCTGAAACGC
GAGAGAAACCGCGTGTCGACTGTGAAACAAAAAACAGCATATTGACGCTG
GGAAAGACCAGAGATCCTGCTGTCTCCTCAGCATCATTCCAGGCACAGAA
CGCCAGAAAATGGAATGGTGCTGTTGAATCAACAGGTTCT.

Any example herein shall be taken to apply mutatis mutandis to any other example unless specifically stated otherwise. For instance, as the skilled person would understand examples of Flaviviridae outlined above for a cell of the invention equally apply to the methods of the disclosure.

### BRIEF DESCRIPTION OF THE DRAWINGS

FIG. 1 Flavivirus genome structure. The genomic polyprotein sequence of a Flavivirus is shown. Structural proteins CprME are located towards the N-terminus. Non-structural proteins NS1 to NS5 are also indicated (source: viralzone.expasy.org).

FIG. 2 provides examples of vectors for expression of the structural proteins. (A-D) shows representative vectors for DENV-2 structural protein (CprME) expression: pSRS11-EF1αFP-DENV-2 CprME (hco)-IRES-mCherry, pSRS11-EF1αFP-DENV-2 CprME (hco)-IRES-mCherry mutant 1, pSRS11-EF1αFP-DENV-2 CprME (hco)-IRES-mCherry mutant 2, and pCDH-EF1αSP-DENV-2 CprME (hco)-Poly A-PGKp-GFP-T2A-puromycin, respectively. (E-H) shows representative vectors for non-structural (NS1˜NS5) expression: pCDH-EF1αSP-DENV-2 NS1˜NS5 (hco)-Poly A-PGKp-GFP-T2A-puromycin, pCDH-EF1αSP-DENV-2 NS1˜NS5 (hco)-Poly A-PGKp-mCherry-T2A-puromycin, pCDH-EF1αSP-DENV-2 CprME (mco)-Poly A-PGKp-GFP-T2A-puromycin, and pCDH-EF1αFP-DENV-2 CprME (mco)-Poly A-PGKp-GFP-T2A-puromycin, respectively. (I-J) Show representative vectors for DI RNA expression: pCDH-CMVp-DENV-2 DI_290-HDVr-Poly A-PGKp-CFP-T2A-puromycin and pCDH-CMVp-DENV-2 DI_290-HDVr-S/MAR-Poly A-PGKp-GFP-T2A-puromycin, respectively. Components of the vectors are described in Tables 1-3.

FIG. 3 provides an example of the dengue DIP production system. Schematics of (A) a Self-inactivating (SIN) lentiviral vector with a codon-optimised gene encoding the non-structural proteins (NS1˜NS5) of DENV serotype 2 (DENV2 NS) and EGFP, (B) a SIN retroviral vector with a codon optimised gene encoding structural proteins of DENV2 (capsid (C), premembrane (prM)/membrane(M) and envelope (E) (CprME)) and mCherry, and C) a lentiviral vector that initiates transcription (black arrow) of a cDNA encoding a DENV2_290 nucleotide DI RNA (DENV2 DI_290) in frame with the hepatitis delta ribozyme that cleaves the DI RNA at the precise 3′ end (blue arrow). D) Lentiviral and retroviral vectors are produced and used to transduce VeroE6 cells that are selected by FACS for high levels of each fluorescent protein. The cells constitutively produce DENV-based DIPs that can be harvested from cell-free culture supernatant.

FIG. 4 shows Vero-D2G2 cells expressing DENV-2 structural and non-structural proteins. Western blots of lysate from Vero-D2G2 cells, Vero cells and DENV-2 infected Vero cells as indicated. Antibodies specific for the DENV-2 NS protein NS5, and the structural proteins E and capsid are shown. The blot was also probed with an antibody to cellular tubulin.

FIG. 5 shows the replication of DI RNA_290 in VeroD2G2 cells compared to controls and the production and transmission of DI RNA by DIPs. (A) shows replication of DI RNA_290 in Vero-D2G2 cells compared to controls. Replication of DENV RNA in cells results in formation of double strand (ds) RNAs. The image displays immuno-staining of Vero cells infected with DENV-2 (top row), Vero-D2G2 cells expressing DI RNA_290 (middle row), and Vero cells expressing DI RNA_290 (bottom row) and unmodified Vero Cells (not shown) with an antibody to ds RNA. Confocal Microscopy of the stained cells shows that ds RNA is detectable in the DENV-2 infected Vero and Vero-D2G2 cells, but not in the other two cell lines. The outcome shows high levels of DI RNA required for DIP production only occurs in Vero-D2G2 cells. (B) & (C) show production and transmission of DI RNA by DIPs. (B) Vero-D2-Gen2 or Vero cells transfected with D2-290nt DI RNA. The data show that DIPs are produced by Vero-D2-Gen2 cells. The DI-RNA detected in Vero supernatant is due to exosome contamination. C) DIPs supernatant or control supernatant were added parental Vero-D2-Gen2 or Vero cells. DI RNA replicates and produce de novo DIPs only in Vero-D2-Gen2 cells.

FIG. 6 displays the mosquito blood feed apparatus used to deliver DENV and/or DIPs. Mosquitos (Aedes aegypti) are infected with DENV in sheep blood which is delivered in a feeding solution via a porcine intestinal membrane. After 7 and 14 days mosquitos are collected and DENV measured in bodies, legs and saliva.

FIG. 7 shows that the 290 DI RNA can inhibit infection of mosquitoes by DENV. Individual mosquitoes were fed with blood meal containing 10⁸ CCID₅₀/ml DENV-2 (QML16 strain) on day 0. Mosquitoes were microinjected with 0.1 μL DENV-2 290 DI RNA (the RNA is in vitro transcribed and purified DI RNA) or control RNA at 2 or 4 days post infection (dpi) (n=10). Mosquitoes were dissected and nucleic acid samples were collected at 14 dpi. Using RT-PCR and primers to the DENV NS5 region of the viral genome, no DENV genomic RNA was detected in mosquito samples microinjected with 290 DI RNA at 2 dpi. The outcome suggests that microinjecting DI RNA into mosquitoes up to two days post infection (dpi) clears virus infection in mosquito bodies by 14 dpi.

FIG. 8 shows that DIPs comprising 290 DI RNA can inhibit replication of DENV1, DENV2, DENV3 and DENV4. Huh7 cells were infected with each DENV serotype at multiplicity of infection (MOI) 1. After 4 h, the virus was removed and DIPs comprising the 290 DI RNA added (equal to 1 DI RNA copy per cell), or cells were not treated, and incubated for 16 h post-infection. The cells were washed with fresh culture medium and incubated for up to 72 h post-infection. A sample of cell-free culture supernatant was assayed for DENV RNA by RT-qPCR using primers to measure DENV-1-DENV-4 NS5 open reading frame (orf). The data shows that DIPs could inhibit replication of each DENV serotype in Huh7 cells.

FIG. 9 shows that D2-290nt inhibited replication of Zika virus (ZIKV) in HuH7 cells. DENV-2 290 DI RNA or control RNA was delivered to human HuH7 cells in triplicate and then infected with ZIKA (MOI of 0.01) for 3 h. DI RNA_D2-290nt was made by in vitro transcription and the purified RNA was transfected into Huh7 cells. Supernatants were collected after 3 days post infection and assayed for levels of ZIKV genomic RNA by RT-qPCR in triplicate.

FIG. 10 shows that DIPS inhibit DENV2 replication in a dose dependent manner. Huh7 cells were infected with DENV-2 at an MOI 1 for 3 h and then cell medium was replaced with fresh culture medium containing DIPs at 0.1, 0.2, 0.4, 0.8, 1.6, 3.2, 6.4 and 12.8 DI 290 RNA copies/cell. At 48 h post-infection, the RNAs from the culture supernatants were extracted and the concentration of DENV2 genomic RNA in culture supernatants were measured by RT-qPCR using oligonucleotide primers to DENV NS5 gene (A). The IC50 was calculated using graph pad prism8 (B).

FIG. 11 shows episomal long-term expression of DI RNA using S/MAR sequence. Huh 7 cells were transfected with pCDH that can produce DI-RNA 290 and also contains the scaffold/matrix attachment region (S/MAR) element. As a control, Huh 7 cells were transfected with the same pCDH plasmid missing the S/MAR genetic element.

FIG. 12 shows that Happy Cell® Advanced Suspension Medium (ASM) improves cell density and DIP production. The effect of ASM's cell culture on DIP production was assessed at two cell densities 4×10⁵ (A) and 8×10⁵ (B). DIP-producing cells were seeded into each well of a non-treated 24 well plate with Happy cell ASM medium (stock:4×) at a final concentration of 1× and 3×ASM, or in normal medium (Cell only samples). On day 3, inactivation solution was added to disrupt the ASM suspension polymer complex and the total cell numbers were determined using a haemocytometer (A, left) and (B, left). The RNAs from the culture supernatants were extracted and subjected to RT-qPCR to measure levels of DI 290 RNA (A, left) and (B, left).

FIG. 13 Temperature-enhanced production of DIPs. HEK 293T and HEK 293T-D2-DI 2 producing cells were seeded into 12-well plates (1.5×10⁵ cells/well) and incubated at 33, 37 and 39° C. The culture supernatants were collected on day 3 post-incubation, the RNA was isolated from the culture supernatant and used in RT-PCR reaction to measure of levels of DENV2 DI RNA.

FIG. 14 shows the procedure for oral infection of mosquitoes and treatment with DI RNAs by intrathoracic microinjection. On day 0, individual mosquitoes were fed with a blood meal containing 10⁸ CCID₅₀/ml DENV-2. On days 2 and 4 post-infection, mosquitoes were microinjected with DENV-2 290 DI RNA or control (scrambled sequence) RNA. At day 14, mosquitoes were dissected and nucleic acid samples collected.

FIG. 15 shows that DIP administration to mosquitoes clears virus infection in mosquito bodies 14 days post infection. DENV genomic RNA was detected in mosquito samples microinjected with 290 DI RNA at 2 dpi as shown in FIG. 13. (A) RT-qPCR and primers to the DENV NS5 region of the viral genome were used to detect DENV-2 infection. The infection rate of mosquitoes is indicated by bars and the number indicates the number of mosquitoes tested. (B) The level of DENV-2 RNA measured in the body sample is shown. Points indicate individual mosquitoes. (C) The level of infectious DENV-2 present samples from legs and wings from individual mosquitoes is shown. Bars indicate medians. LOD, limit of detection.

FIG. 16 shows the expression of DENV2 structural and non-structural proteins in HEK 293T cells. Vectors were used to deliver the dengue viral structural protein open reading frame (ORF) and non-structural ORF. The expression of DENV2 mRNA in the DIP-producing cell line was measured by RT-qPCR using oligonucleotide primers to DENV2 E, NS1 and NS5 gene (A, top). Moreover, the expression and cellular distribution of viral proteins were confirmed by western blot (A, bottom) and immunofluorescence (B-D) analyses using anti-E, anti-CA, anti-NS3 and anti-NS5 antibodies.

FIG. 17 shows the stable expression of DENV DI RNA in HEK 293T cells expressing DENV2 structural and non-structural proteins. A vector containing a specific DENV DI gene was introduced into the DIP-producing cell line to continually express DENV DI RNA. The expression of DI RNA in the cells was confirmed by RT-qPCR using primers to DI RNA (A). By using the antibody directed against dsRNA, dsRNA was detectable in the DIP-producing cells (B, middle row) and DENV2-infected cells (2B, Bottom row).

FIG. 18 shows DIP purification. (A) Culture supernatants from DIP-producing cells were subjected to velocity gradient (5-50% sucrose). Fractions were collected from the bottom and were assayed for DI 290 RNA copy number by RT-qPCR and DENV2 E protein by dot blot analysis with an anti-E antibody. The supernatants from DENV2-infected cells and cells stably expressing only DENV2 proteins (DENV2 ORF) were included as controls. (B) Culture supernatants from the DIP-producing cells were loaded onto the CHT ceramic hydroxyapatite column and eluted with sodium phosphate buffer. (C) The CHT purified supernatants were further applied to a membrane filter device. Samples of the CHT purified supernatant, the concentrated supernatant and the flow through supernatants were ultra-centrifuged. The RNA was extracted from the pelleted material and used in RT-qPCR to measure the levels of DI RNA.

FIG. 19 shows that D2 DI290 DIPs stimulate the MX-A interferon inducible innate immunity factor encoded by the MX-1 gene. The graph shows that MX-1 mRNA is highly elevated in uninfected and DENV-infected Huh7 cells treated by DENV-2 290 DI RNA (D2-290nt) DIPS but not by negative control DIPs (Neg. Ctrl. DIPs). Huh7 cells were untreated (first lane) or treated with D2-290nt DIPs. (second lane) or with Neg. Ctrl. DIPs that have no antiviral activity (third lane). Otherwise Huh7 cells were infected with DENV-2 at an MOI of 1.0 for 2 hours and then the virus was removed. Samples included DENV-2 infected only cells (fourth lane), infected cells treated with D2-290nt DIPs (fifth lane) or treated with Neg. Ctrl. DIPs (last lane). Cellular RNA was collected after 48 h post-infection for all samples that were assayed by RT-qPCR to measure the level of MX-1 mRNA. The MX-1 levels were normalised to levels of cellular GAPDH in the same sample.

FIG. 20 shows that D2-DI290 DI RNA can inhibit ZIKV RNA levels secreted by infected Huh7 cells. A) ZIKV MOI of 0.01 or B) MOI of 0.1 were used to infect Huh7 cell. Mean values and errors bars are shown. A two-tailed Student T-Test with equal variance was used to calculate P values.

### KEY TO SEQUENCE LISTING

SEQ ID NO:1: DENV2 CprME nucleic acid sequence human codon optimized

SEQ ID NO:2: DENV2 NS1-5 nucleic acid sequence human codon optimized

SEQ ID NO:3: DENV2 NS1-5 nucleic acid sequence old word monkey codon optimized

SEQ ID NO:4: nucleic acid sequence partial eF1 alpha promoter

SEQ ID NO:5: nucleic acid sequence full length eEf1 alpha promoter

SEQ ID NO:6: nucleic acid sequence for primer D2-C-opt-T2A-Xma1-For

SEQ ID NO:7: nucleic acid sequence for primer D2-E-opti-Ecor1-T2A-Rev

SEQ ID NO:8: nucleic acid sequence for primer D2-NS1-Ecor1-For

SEQ ID NO:9: nucleic acid sequence for primer D2-NS5-BamH1-Rev

SEQ ID NO:10: nucleic acid sequence for primer pCFP-coilin For

SEQ ID NO:11: nucleic acid sequence for primer pCFP-coilin Rev

SEQ ID NO:12: nucleic acid sequence for primer pCDH-EF1α-MCS-BGH-PGK-T2A-Puro For

SEQ ID NO:13: nucleic acid sequence for primer pCDH-EF1α-MCS-BGH-PGK-T2A-Puro Rev

SEQ ID NO:14: nucleic acid sequence for primer human codon optimisied E gene For

SEQ ID NO:15: nucleic acid sequence for primer human codon optimisied E gene Rev

SEQ ID NO:16: nucleic acid sequence for primer monkey codon optimisied NS1 gene For

SEQ ID NO:17: nucleic acid sequence for primer monkey codon optimisied NS1 gene Rev

SEQ ID NO:18: nucleic acid sequence for NS5 gene For

SEQ ID NO:19: nucleic acid sequence for NS5 gene Rev

SEQ ID NO:20: nucleic acid sequence for primer monkey codon optimised NS5 gen For

SEQ ID NO:21: nucleic acid sequence for primer monkey codon optimised NS5 gen Rev

SEQ ID NO:22: nucleic acid sequence for primer quantification DENV DI RNA For

SEQ ID NO:23: nucleic acid sequence for primer quantification DENV DI RNA Rev

SEQ ID NO:24: nucleic acid sequence for primer GAPDH For

SEQ ID NO:25: nucleic acid sequence for primer GAPDH Rev

SEQ ID NO:26: vector nucleic acid sequence for expression of DENV-1 DI-RNA 443

SEQ ID NO:27: vector nucleic acid sequence for expression of DENV-2 DI-RNA 290

### DETAILED DESCRIPTION OF THE INVENTION

### General Techniques and Selected Definitions

The term “and/or”, e.g., “X and/or Y” shall be understood to mean either “X and Y” or “X or Y” and shall be taken to provide explicit support for both meanings or for either meaning.

As used herein, the terms “a”, “an” and “the” include both singular and plural aspects, unless the context clearly indicates otherwise.

Any discussion of documents, acts, materials, devices, articles or the like which has been included in the present specification is not to be taken as an admission that any or all of these matters form part of the prior art base or were common general knowledge in the field relevant to the present disclosure as it existed before the priority date of each claim of this application.

Throughout this specification, unless specifically stated otherwise or the context requires otherwise, reference to a single step, composition of matter, group of steps or group of compositions of matter shall be taken to encompass one and a plurality (i.e. one or more) of those steps, compositions of matter, groups of steps or group of compositions of matter.

Each example described herein is to be applied mutatis mutandis to each and every other example of the disclosure unless specifically stated otherwise.

Those skilled in the art will appreciate that the disclosure is susceptible to variations and modifications other than those specifically described. It is to be understood that the disclosure includes all such variations and modifications. The disclosure also includes all of the steps, features, compositions and compounds referred to or indicated in this specification, individually or collectively, and any and all combinations or any two or more of said steps or features.

The present disclosure is not to be limited in scope by the specific examples described herein, which are intended for the purpose of exemplification only. Functionally-equivalent products, compositions and methods are clearly within the scope of the disclosure.

The present disclosure is performed without undue experimentation using, unless otherwise indicated, conventional techniques of molecular biology, recombinant DNA technology, cell biology and immunology. Such procedures are described, for example, in Sambrook, Fritsch & Maniatis, Molecular Cloning: A Laboratory Manual, Cold Spring Harbor Laboratories, New York, Second Edition (1989), whole of Vols I, II, and III; DNA Cloning: A Practical Approach, Vols. I and II (D. N. Glover, ed., 1985), IRL Press, Oxford, whole of text; Oligonucleotide Synthesis: A Practical Approach (M. J. Gait, ed, 1984) IRL Press, Oxford, whole of text, and particularly the papers therein by Gait, ppl-22; Atkinson et al, pp 35-81; Sproat et al, pp 83-115; and Wu et al, pp 135-151; 4. Nucleic Acid Hybridization: A Practical Approach (B. D. Hames & S. J. Higgins, eds., 1985) IRL Press, Oxford, whole of text; Immobilized Cells and Enzymes: A Practical Approach (1986) IRL Press, Oxford, whole of text; Perbal, B., A Practical Guide to Molecular Cloning (1984); Methods In Enzymology (S. Colowick and N. Kaplan, eds., Academic Press, Inc.), whole of series, Sakakibara, D., Teichman, J., Lien, E. Land Fenichel, R. L. (1976). Biochem. Biophys. Res. Commun. 73 336-342; Merrifield, R. B. (1963). J. Am. Chem. Soc. 85, 2149-2154; Barany, G. and Merrifield, R. B. (1979) in The Peptides (Gross, E. and Meienhofer, J. eds.), vol. 2, pp. 1-284, Academic Press, New York. 12. Wünsch, E., ed. (1974) Synthese von Peptiden in Houben-Weyls Metoden der Organischen Chemie (Müler, E., ed.), vol. 15, 4th edn., Parts 1 and 2, Thieme, Stuttgart; Bodanszky, M. (1984) Principles of Peptide Synthesis, Springer-Verlag, Heidelberg; Bodanszky, M. & Bodanszky, A. (1984) The Practice of Peptide Synthesis, Springer-Verlag, Heidelberg; Bodanszky, M. (1985) Int. J. Peptide Protein Res. 25, 449-474; Handbook of Experimental Immunology, Vols. I-IV (D. M. Weir and C. C. Blackwell, eds., 1986, Blackwell Scientific Publications); and Animal Cell Culture: Practical Approach, Third Edition (John R. W. Masters, ed., 2000), ISBN 0199637970, whole of text.

Throughout this specification, unless the context requires otherwise, the word “comprise”, or variations such as “comprises” or “comprising”, will be understood to imply the inclusion of a stated step or element or integer or group of steps or elements or integers but not the exclusion of any other step or element or integer or group of elements or integers.

Unless specifically defined otherwise, all technical and scientific terms used herein shall be taken to have the same meaning as commonly understood by one of ordinary skill in the art (for example, in cell culture, molecular genetics, immunology, immunohistochemistry, protein chemistry, and biochemistry).

The term “consists of” or “consisting of” shall be understood to mean that a method, process or composition of matter has the recited steps and/or components and no additional steps or components.

The term “about”, as used herein when referring to a measurable value such as an amount of weight, time, dose, etc. is meant to encompass variations of ±20% or ±10%, more preferably ±5%, even more preferably ±1%, and still more preferably ±0.1% from the specified amount, as such variations are appropriate to perform the disclosed method.

A codon is a sequences of three nucleotides which together form a unit of a genetic code in a RNA or DNA molecule. There is redundancy in the code, in that more than one codon encodes a specific amino acid. Therefore, one polypeptide chain can be encoded by a number of different amino acid sequences. The usages of specific codons can influence gene expression. Different organisms have a bias towards which codons they use to encode an amino acid. For example, humans have specific codons that they use more frequently to encode particular amino acids. As used herein “codon optimised” refers to optimising the codons of an RNA a DNA molecule for increasing expression of a RNA or DNA molecule in a cell described herein. Codon optimisation can include optimising the RNA or DNA molecule to comprise codons that occur more frequently in another organism e.g. humans or Old World monkeys. This can include, for example, removing rare codons that are rate-limiting for protein synthesis in a particular cell with frequently used codons in a particular cell to increase expression. This may also include replacing synonymous codons, codons that are interchangeable without affecting protein structure and function, with codons that are used more frequently in a particular cell. In one example, an RNA or a DNA molecule may be optimised for expression with human codons. In one example, an RNA or a DNA molecular may be optimised for expression with Old World monkey codons. In one example, an RNA or a DNA molecular may be optimised for expression with avian codons.

By “corresponding native virus” or “wild type virus” or “parental virus” it is meant a virus that comprises the complete genomic sequence encoding for viral structural and non-structural proteins as well as all the genomic elements required for replication and packaging.

As used herein, the term “subject” is any animal. The term includes any human or non-human animal. For example, the animal is a mammal, avian, arthropod, chordate, amphibian or reptile. Exemplary subjects include but are not limited to human, primate, livestock (e.g. sheep, cow, chicken, horse, donkey, pig), companion animals (e.g. dogs, cats), laboratory test animals (e.g. mice, rabbits, rats, guinea pigs, hamsters), captive wild animal (e.g. fox, deer). In one example, the animal is a mammal. In one example, the mammal is a human. In one example, the animal is an avian. In one example, the subject is a Flaviviridae host.

As used herein, a “host” is an organism that an infectious form of the virus can replicate within. Replication of the virus within a host can result in disease in the host.

As used herein, a “carrier” or “vector” refers to an organism in which an infectious form a virus can replicate but in which no signs and symptoms of the disease are displayed. A carrier can transmit the virus to other organisms susceptible to infection with the virus.

As used herein an “antiviral effect” refers to killing a virus, inhibiting a virus, reducing the replicating or a virus or reducing the transmission of a virus. In one example, the antiviral effect is an immune response. In one example, the immune response is an interferon response. In one example, the immune response is an antibody response. In one example, the antiviral effect is viral interference.

As used herein “viral interference” refers to where virus replication is supressed in a cell due to a reduction in the availability of the host replication machinery and/or viral replication machinery to produce the virus. For example, viral interference may be caused by the presence of one or more additional virus or the presence of one or more DI RNA competing for host replication machinery and/or viral replication machinery.

As used herein, the term “treating”, “treatment” or “treats” includes alleviation of one or more symptoms associated with a disease or condition. For example, as used herein, the term “treating a Flaviviridae disease” includes alleviating one or more symptoms associated with a Flaviviridae disease. In one example, the term “treating a Flaviviridae disease” refers to a reduction in viral load in a subject. In one example, the term “treating a Flaviviridae disease” refers to a decrease in the period of illness associated with a Flaviviridae disease. “Treatment” can also mean prolonging survival as compared to expected survival if not receiving treatment. In one example, treating includes administering a effective amount of a DIP, or composition as described herein sufficient to reduce or eliminate at least one symptom of a specified disease or condition.

As used herein, the term “prevention” or “preventing includes prophylaxis of the specific disease or condition. For example, as used herein, the term “preventing a Flaviviridae disease” refers to preventing the onset or duration of one or more of the symptoms associated with a Flaviviridae disease. In one example, the term “preventing a Flaviviridae disease” refers to inhibiting viral replication (reducing viral load) in a subject that has been exposed to a Flaviviridae disease. In one example, the term “preventing a Flaviviridae disease” refers to slowing or halting the progression of a Flaviviridae disease. In one example, the term “preventing a Flaviviridae disease” refers to preventing a congenital disease caused by infection of a subject's mother with a Flaviviridae disease before or during pregnancy. In one example, preventing includes administering an effective amount of a DIP or composition as described herein sufficient to stop or hinder the development of at least one symptom of a specified disease or condition.

Reference to a “single round of infection” refers to a virus which has been genetically compromised such that following a single infection into a host cell, it is not capable of using the host cell machinery to generate further virus particles. Typically, such virus will include the genomic elements required for replication and packaging (e.g. encapsulation sequence) but lack at least the structural proteins (CprME) required to produce complete virus particles. Such viruses can replicate when supplemented with the lacking components. For example, such viruses can replicate in the presence of wild-type virus.

### Flaviviridae

Flaviviridae is a family of small enveloped viruses was genomes of approximately 9000 to 13,000 nucleotides. The genomes of Flaviviridae are RNA positive-stranded. The Flaviviridae may be distributed by an arthropod carrier, including for example mosquitoes and ticks.

A person skilled in the art will appreciate that the i) structural proteins of a virus of the Flaviviridae family i) non-structural proteins of the Flaviviridae family and that the Flaviviridae defective interfering genomic sequence can be from any Flaviviridae virus known to a person skilled in the art.

In one example, the Flaviviridae is selected from: Flavivirus, Hepacivirus, Pegivirus, Pestivirus, and Jingmenvirus.

In one example, the Flaviviridae is a Flavivirus.

The Flaviviridae may be selected from, for example, Dengue virus (DENV), West Nile virus (WNV), Yaounde virus, Yellow fever virus (YFV), Zika virus (ZIKA), Apoi virus, Aroa virus, Bagaza virus, Banzi virus, Bouboui virus, Bukalasa bat virus, Cacipacore virus, Carey Island virus, Cowbone Ridge virus, Dakar bat virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Penh bat virus, Powassan virus, Rio Bravo virus, Royal Farm virus, Saboya virus, Sal Vieja virus, San Perlita virus, Saumarez Reef virus, Sepik virus, St. Louis encephalitis virus, Tembusu virus, Tick-borne encephalitis virus, Tyuleniy virus, Uganda S virus, Usutu virus, Wesselsbron virus, and Yokose virus.

In one example, the host of the Flaviviridae is an animal. In one example, the host of the Flaviviridae is a mammal. In one example, the host of the Flaviviridae is an avian. In one example, the host of the Flaviviridae is a human. In one example, the host of the Flaviviridae is a primate. In one example, the host of the Flaviviridae is a monkey. In one example, the host of the Flaviviridae is a rodent. In one example, the host of the Flaviviridae is a livestock animal e.g. sheep, horse, cow, pig, ruminant, dog, chicken, duck, turkey, and quail.

Flaviviridae with human hosts include, for example, DENV, WNV, YFV, ZIKA, Japanese encephalitis virus, Murray Valley encephalitis virus, Usutu virus, and Tick-borne encephalitis virus. In one example, the Flavivirus is ZIKA. In one example, ZIKA comprises the sequence of GenBank accession no. KX893855.1. In one example, the Flavivirus is ZIKA. In one example ZIKA comprises the sequence of GenBank accession no. KX702400.1

In one example, the Flavivirus is WNV. In one example, WNV comprises the sequence of NBI Reference sequence NC_001563.2. In one example, WNV comprises the sequence of NBI Reference sequence NC_009942.1

In one example, the Flavivirus is YFV. In one example, YFV comprises the sequence of NBI Reference sequence NC_002031.1.

As used herein, “Flaviviridae disease” refers to a disease in a host caused by a Flaviviridae virus as described herein. In one example, the disease is selection from one or more of: fever, rash, myalgia, haemorrhagic fever, abortion, encephalitis, neonatal encephalitis, egg-drop-syndrome, neuroparalytic disease, myocardial necrosis, hepato- and splenomegaly, congenital disease, acute dengue disease, severe dengue disease and severe dengue disease caused by antibody dependent enhancement.

Examples of symptoms caused by a Flaviviridae include one or more of: fever, rash, headache, fatigue, muscle pain, joint pain, pain behind the eyes, nausea, vomiting, nose bleeding, bleeding gums, easy bruising, bleeding under the skin, bleeding in internal organs, and bleeding from bodily orifices (e.g. mouth, eyes or ears).

In one example, the Flaviviridae disease is selected from one or more of: acute dengue disease, severe dengue disease and severe dengue disease caused by antibody dependent enhancement.

### Flaviviridae Structural and Non-structural Proteins and Replication

Viruses of this family are enveloped, spherical and approximately 50 nm in diameter. The surface proteins (E dimer and M protein) are arranged in an icosahedral-like symmetry.

The genomes of many Flaviviridae, in particular Flaviviruses, comprise a long open reading frame encoding a polyprotein that is co- and post-translationally processed through cellular and viral proteases into three structural proteins (C, prM, and E) and seven non-structural (NS1, NS2A, NS2B, NS3, NS4A, NS4B, and NS5) proteins flanked by 5′ and 3′ terminal non-coding regions. The structural proteins are required for defective interfering particle (DIP) formation and the non-structural proteins are required for replication. The 5′ and 3′ terminal non coding regions play a role in viral translation and replication.

A reference to “structural proteins” as described herein refers to one or more of the three structural proteins: capsid (C), premembrane (prM)/membrane (M), and envelope (E). In one example, a reference to “structural proteins” is a reference to C, prM/M and EA. In one example, reference to “non-structural proteins” as described herein refers to one or more or all of the non-structural proteins in the Flaviviridae genome. In one example, a reference to “non-structural proteins” is a reference to NS1, NS2A, NS2B, NS3, NS4A, NS4B, and NS5.

According to the disclosure, the aforementioned structural and non-structural proteins may be from any member of the Flaviviridae. In one example, the structural proteins and non-structural proteins are from the same Flaviviridae. In one example, the structural and non-structural proteins are from different Flaviviridae. Preferably, the structural proteins and non-structural proteins are from Dengue virus (DENV). In one example, the structural and non-structural proteins are from different DENV serotypes. In one example, the structural and non-structural proteins are from different DENV serotypes. More preferably, the structural proteins and non-structural proteins are from DENV2.

In one example, the structural proteins are from more than one Flaviviridae. In one example the non-structural proteins are from more than one Flaviviridae. In one example, the structural proteins are from one Flaviviridae and the non-structural proteins are from another Flaviviridae.

In one example, the structural proteins are from more than one DENV serotype. In one example the non-structural proteins are from more than one DENV serotype. In one example, the structural proteins are from one DENV serotype and the non-structural proteins are from another DENV serotype.

In one example, the structural proteins and/or non-structural proteins are codon optimised. In one example, the structural proteins and/or non-structural proteins are human or Old World monkey codon optimised. In one example, one of the structural proteins and non-structural proteins are human codon optimised and one of the structural proteins and/or non-structural proteins. In one example, the structural proteins are encoded by a sequence comprising or consisting of SEQ ID: 1. In one example, the non-structural proteins are encoded by a sequence comprising or consisting of SEQ ID NO: 2 or SEQ ID NO: 3.

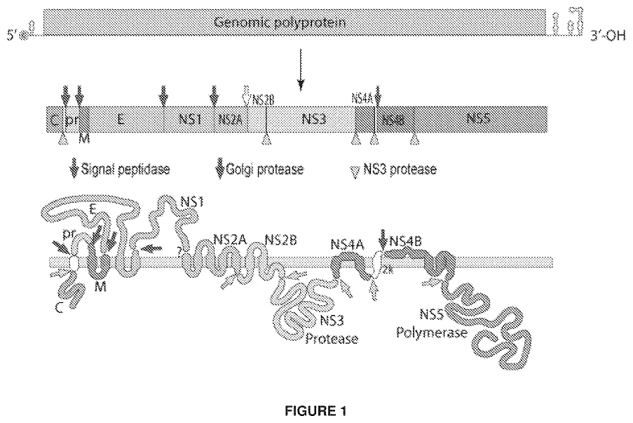

FIG. 1 provides a representation of the organisation of the structural genome of a Flavivirus. The genome is a nonpartite, linear ssRNA(+) genome of 10-11 kb. The 5′ end of the genome has a methylated nucleotide cap for canonical cellular translation. The 3′ terminus is not polyadenylated but forms a loop structure. This secondary structure leads to the formation of a subgenomic flavivirus RNA (sfRNA) through genomic RNA degradation by host 5′-3′ exoribonuclease 1 (XRNA1). sfRNA is essential for pathogenicity and may play a role in inhibiting host RIG-1 antiviral activity (Manokaran G et al (2015) Science 9; 350(6257):217-21).

The virion RNA serves as both the genome and the viral messenger RNA. The whole genome is translated in a polyprotein which is processed co- and post-translationally by host and viral proteases.

Viral replication first involves attachment of the viral envelop protein E to host receptors which mediates internalisation into the host cell by clathrin-mediated endocytosis or apoptotic mimicry. Following fusion of the virus membrane with the host endosomal membrane, the viral RNA genome is released into the cytoplasm. The positive-sense genomic ssRNA is translated into a polyprotein which is cleaved into all structural and non-structural proteins. A double stranded (dsRNA) genome is synthesised from the genomic ssRNA(+). The dsRNA genome is transcribed to provide new viral ssRNA(+) genomes. Virus assembly then occurs in the endoplasmic reticulum. The virion buds at the endoplasmic reticulum and is transported to the Golgi apparatus. The prM protein is cleaved in the Golgi thereby maturing the virion and release of new virions occurs by exocytosis.

### Defective Interfering (DI RNA) Particles

As used herein, a “defective interfering genomic sequence” also referred to as a “defective interfering RNA” or “DI RNA” refers to a partial viral genome that lacks the capacity to code for all the necessary components required for independent replication. They refer to a class of viruses that are defective because they have lost a portion of their genome that encodes a function required for production of complete virus particles (e.g. structural proteins). In one example, the DI RNA comprises all the genomic elements required for replication and packaging by viral structural and non-structural proteins as well as viral genomic sequence (e.g. RNA) comprising a deletion relative to a corresponding native infectious virus. Typically, the genomic deletion may comprise between about 75 and 98%, preferably between about 80 and 90% or about 85 to 90% of the genome.

DIPs occur naturally in nature and can replicate with supplementation of the lost function. For example, DIPs can replicate when in the presence of a wild type virus which provides the missing function or when certain proteins are provided in trans.

The DI RNA sequences described herein are preferably expressed from a cDNA.

In certain examples, the DI RNA sequences are short fragments of Dengue virus RNA containing only key regulatory elements at the 3′ and 5′ ends of the genome. In a particular example, the DI RNA comprises a sequence modification of the genome compared to the native corresponding virus. In one example, the modification is an internal deletion of the genomic sequence. In some examples, the internal deletion may comprise between about 75 and 98%, preferably between about 80 and 90% or about 85 to 90% of the genome.

In other examples, the DI RNA is a naturally occurring DI RNA identified in a host or carrier infected with a wild type virus. A person skilled in the art will appreciate that the naturally occurring Flaviviridae DI RNA may be any Flaviviridae DI RNA previously described in the literature including for example the DI RNA described in Salas-Benito and De Nova-Ocampo et al., (2015), Li et al., (2011), Li et al., (2014).

In one example, the Flaviviridae DI RNA is a naturally occurring DI RNA described above that has been modified to influence one or more properties. For example, the naturally occurring DI RNA has been modified to increase expression or stability of the DI RNA.

In one example, the Flaviviridae DI RNA is encoded by a sequence selected from SEQ ID NO:26 or SEQ ID NO:27. In another example, the Flaviviridae DI RNA is encoded by a sequence selected from one or more of SEQ ID NO:28 to SEQ ID NO:41.

In one example, the Flaviviridae DI RNA is a dengue virus DI RNA (DENV DI RNA). In one example, the DENV DI RNA is selected from a: DENV1 DI RNA, DENV2 DI RNA, DENV3 DI RNA, and DENV4 DI RNA.

In one example, the DENV DI RNA is a DENV1 DI RNA. In one example, the DENV1 DI RNA is encoded by the sequence set forth in SEQ ID NO:25

In one example, the DENV DI RNA is a DENV2 DI RNA. In one example, the DENV2 DI RNA is encoded by the sequence set forth in SEQ ID NO:26.

In one example, the DENV DI RNA is a DENV3 DI RNA.

In one example, the DENV DI RNA is a DENV4 DI RNA.

In certain examples, the disclosure provides a cloned or recombinant virus defective interfering particle (DIP) expressed by the cell line as described herein, or produced by the method as described herein.

In an aspect, the disclosure provides an isolated virus defective interfering particle (DIP) expressed by the cell line as described herein, or produced by the method as described herein.

In one example, the DIP as described herein has an antiviral effect against more than one virus and/or more than one subtype of a virus.

In one example, the DIP as described herein has an antiviral effect against one or more of: i) an RNA virus; ii) a single stranded RNA virus; iii) a positive single stranded RNA virus; iv) a Flaviviridae; v) an Alphavirus; and vi) an Orthopneumovirus.

In one example, the DIP has an antiviral effect against one or more Flaviviridae.

In one example, the Flaviviridae is selected from one or more of DENV, WNV, YFV, ZIKA, Japanese encephalitis virus, Murray Valley encephalitis virus, Usutu virus, and Tick-borne encephalitis virus. In one example, the Flaviviridae is DENV. In one example, the DENV is selected from one or more or all of: DENV1, DENV2, DENV3, and DENV4

In one example, the Alphavirus is chikununyarespiratory syncytial virus.

In one example, the Orthopneumovirus is respiratory syncytial virus.

In one example, the DIP can bind and enter a viral host or viral carrier cell not infected with a corresponding wild type virus.

In one example, the DIP can bind, enter and replicate in a viral host or viral carrier cell comprising a corresponding wild type virus.

In one example, the DIP can bind and enter a Flaviviridae host or Flaviviridae carrier cell not infected with a wild type Flaviviridae virus.

In one example, the DIP can bind, enter and replicate in a Flaviviridae host or Flaviviridae carrier cell comprising a wild type Flaviviridae virus.

### Vectors

Methods for inserting nucleic acid sequences into vectors will be apparent to the skilled person and are described, for example, in Ausubel F. M., 1987 including all updates until present; or Sambrook and Green, 2012. For example, each of the nucleic acids for insertion can be amplified from a suitable template nucleic acid using, for example, PCR and subsequently cloned into a suitable vector. A person skilled in the art would appreciate that a DI RNA as described herein may be cloned into the vector as DNA which is transcribed into RNA. Thus, the term “DI RNA” is a reference to the expressed viral genome sequence.

Means for introducing a vector into a cell for expression are known to those skilled in the art. The technique used for a given organism depends on the known successful techniques. Means for introducing recombinant DNA into cells include microinjection, transfection mediated by DEAE-dextran, transfection mediated by liposomes such as by using lipofectamine (Gibco, Md., USA) and/or cellfectin (Gibco, Md., USA), PEG-mediated DNA uptake, electroporation and microparticle bombardment such as by using DNA- coated tungsten or gold particles (Agracetus Inc., Wis., USA) amongst others.

Vectors suitable for use in the methods of the disclosure include lentiviral, retroviral, adenoviral, herpes virus, adeno-associated viruses and episomal vectors known in the art.

Non-viral vectors include plasmids, episomal vectors, transposon-modified polynucleotides (such as the MVM intron), lipoplexes, polymersomes and combinations thereof. The skilled person will be aware of additional vectors and sources of such vectors.

Lentiviral vector systems have also been developed for construct delivery. Widely used lentiviral vectors in include those based upon human immunodeficiency virus (HIV), feline immunodeficiency virus (FIV), simian immunodeficiency virus (SIV), bovine immunodeficiency virus (BIV), equine infectious anemia virus (EIAV). In one example, the lentival vector is a second generation lentiviral vector. In one example, the lentiviral vector is a third generation lentiviral vector. In one example, the lentiviral vector is pCDH which is available from commercial sources e.g. Addgene. In one example, the lentiviral vector is a pCDH-EF1α vector. In one example, the lentiviral vector is pCDH-EF1α-MCS-BGH-PGK-GFP-T2A-Puro Cloning and Expression Lentivector (SBI System Biosciences). In one example, the lentiviral vectors have a preference for insertion into the host genome in exons.

For example, a retroviral vector generally comprises cis-acting long terminal repeats (LTRs) with packaging capacity for up to 6-10 kb of foreign sequence. The minimum cis-acting LTRs are sufficient for replication and packaging of a vector, which is then used to integrate the expression construct into the target cell to provide long term expression. Widely used retroviral vectors include those based upon y-retroviral vector, murine leukemia virus (MuLV), gibbon ape leukemia virus (GaLV), simian immunodeficiency virus (SrV), human immunodeficiency virus (HIV), and combinations thereof (see, e.g., International publication WO1994/026877; Buchschacher and Panganiban, 1992; Johann et al., 1992; Sommerfelt and Weiss, 1990; Wilson et al., 1989; Miller et al., 1991; Lynch, et al., 1991; Miller and Rosman, 1989; Miller, 1990; Scarpa et al., 1991; Burns et al., 1993). In one example, the retroviral vector is pSRS11. In one example, the retroviral vector preferentially insert vectors into the host genome upstream of promoters.

Various adeno-associated vir (AAV) vector systems have also been developed for nucleic acid delivery. AAV vectors can be readily constructed using techniques known in the art. (see, e.g., U.S. Pat. Nos. 5,173,414 and 5,139,941; International publications WO 92/01070 and WO 93/03769; Lebkowski et al., 1988; Vincent et al., 1990; Carter, 1992; Muzyczka, 1992; Kotin, 1994).

Additional viral vectors useful for delivering an expression construct of the invention include, for example, those derived from the pox family of viruses, such as vaccinia virus and avian poxvirus or an alphavirus or a conjugate virus vector (e.g., that described in Fisher-Hoch et al., 1989).

The vectors according to the present disclosure may comprise one or more of a psi packaging signal (Ψ) sequence, a rev response element (RRE), a promoter, a heterologous sequence, an antibiotic resistance gene, a selectable marker, a response element, central polypurine tract (cPPT), and 3′ and 5′ long terminal repeat (LTR) sequences. In other examples, the vector may include a scaffold/matrix attachment region (S/MAR) sequence (Verghese et al., 2014).

The promoter may be a constitutive or non-constitutive promoter. In some examples, the promoter is a mammalian promoter. The promoter may be selected from a cytomegalovirus immediate early (CMV) promoter, Human elongation factor-1 alpha (EF1α) promoter, a murine stem cell virus (MSCV) promoter, a phosphoglycerate kinase 1 (PGK) promoter, a human Ubiquitin C (UbC) promoter, or a simian virus 40 (SV40) early promoter.

In one example, the vector comprises a Woodchuck hepatitis virus post-transcriptional regulatory element (WPRE). Without wishing to be bound by theory, the WPRE sequence is said to stimulate the expression of transgenes via increased nuclear export.

In one example, the 5′ and/or 3′ LTR comprises a deletion within the LTR. This deletion may comprise part or all of the U3, R or U5 sequence. In a particular example the deletion is a part or all of the U3 region.

Antibiotic resistance genes are known in the art. Non-limiting examples include puromycin, kanamycin, spectinomycin, streptomycin, ampicillin, carbenicillin, bleomycin, erythromycin, polymyxin B, tetracycline, and chloramphenicol.

Selectable markers are also known in the art. Typically, the selectable marker is a fluorescent protein. Suitable selectable markers according to the disclosure include mCherry, Green fluorescent protein (GFP), cyan fluorescent protein (CFP), red fluorescent protein (RFP), tdTomato and mOrange.

It will be appreciated by persons skilled in the art that the vector may be modified to improve efficiency of expression of the heterologous sequence or to improve post-translational modifications.

In one, example the first, second and third vectors as described herein are retroviral and/or lentiviral vectors or a combination thereof. In another example, the first and third vectors are lentiviral vector. In yet another example, the second vector is a retroviral vector.

In one example, the first, second and third vectors are integrated into the host genome. The first and second vector preferentially integrate in different regions of the host genome.

In one example, the first and second vectors are separate vectors.

In one example, the vector preferentially integrates into an exome of the host cell.

In another example, the first, second and/or third vectors are self-inactivating (SIN) vectors.

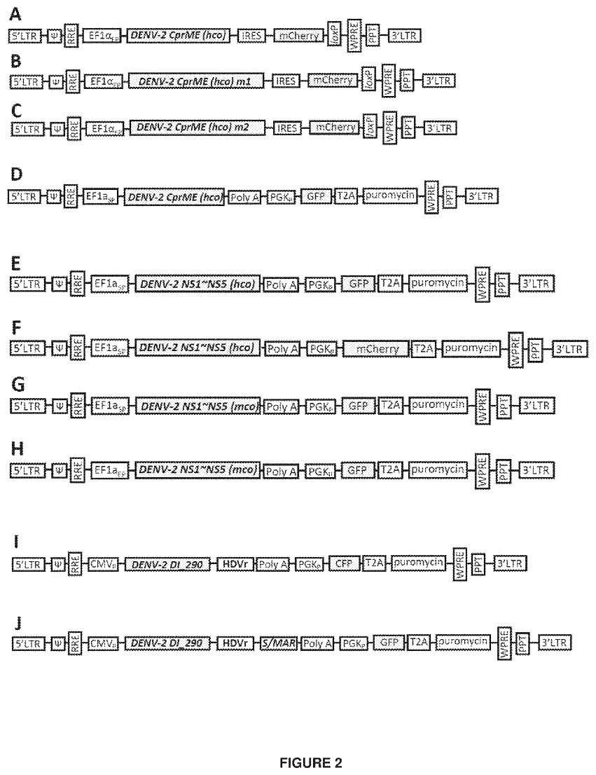

Table 1 provides examples of representative vectors for expression of Dengue virus structural proteins. These are also shown pictorially in FIG. 2A-D.

TABLE 1
Example vectors for DENV structural protein expression

| Name | Description |
|---|---|
| pSRS11-EF1α_{FP}-DENV-2 CprME | Gammaretroviral vector containing |
| (hco)-IRES-mCherry | human codon optimized DENV-2 |
| | CprME gene, IRES sequence and |
| | mCherry sequence. |
| pSRS11-EF1α_{FP}-DENV-2 CprME | Gammaretroviral vector containing |
| (hco)-IRES-mCherry mutant 1 | human codon optimized DENV-2 |
| | CprME gene with F108A mutation in E |
| | sequence, IRES and mCherry sequence. |
| pSRS11-EF1α_{FP}-DENV-2 CprME | Gammaretroviral vector containing |
| (hco)-IRES-mCherry mutant 2 | human codon optimized DENV-2 |
| | CprME gene with F108A mutation in E |
| | and DENV-1 E sequence from 297-495 |
| | aa. |
| pCDH-EF1α_{FP}-DENV-2 CprME | Dual promoter lentivector containing |
| (hco)-Poly A-PGKₚ-GFP-T2A- | human codon optimized DENV-2 |
| puromycin | CprME gene and using a PGK |
| | promoter to drive co-expression of GFP |
| | & Puro. |

FP: full-length promoter;
SP: short promoter;
hco: human codon optimisation

Table 2 provides examples of representative vectors for expression of Dengue virus non-structural proteins. These are also shown pictorially in FIG. 2 (E-H).

TABLE 2
Example vectors for DENV non-structural protein expression

| Name | Description |
|---|---|
| pCDH-EF1α_{FP}-DENV-2 NS1~NS5 | Dual promoter lentivector containing |
| (hco)-Poly A-PGKp-GFP-T2A- | human codon optimized DENV-2 |
| puromycin | non-structural (NS) gene and using a |
| | PGK promoter to drive co-expression |
| | of GFP & Puro. |
| pCDH-EF1α_{FP}-DENV-2 NS1~NS5 | Dual promoter lentivector containing |
| (hco)-Poly A-PGKₚ-mCherry-T2A- | human codon optimized DENV-2 NS |
| puromycin | gene and using a PGK promoter to |
| | drive co-expression of mCherry & |
| | Puro. |
| pCDH-EF1α_{FP}-DENV-2 CprME | Dual promoter lentivector containing |
| (mco)-Poly A-PGKp-GFP-T2A- | monkey codon optimized NS gene |
| puromycin | and using a PGK promoter to drive |
| | co-expression of GFP & Puro. |
| pCDH-EF1α_{FP}-DENV-2 CprME | Dual promoter lentivector using a |
| (mco)-Poly A-PGKₚ-GFP-T2A- | full-length EF1α promoter to express |
| puromycin | monkey codon optimized DENV-2 |
| | NS protein and a PGK promoter to |
| | drive co-expression of GFP & Puro. |

FP: full-length promoter;
SP: short promoter;
hco: human codon optimisation;
mco: monkey codon optimisation

Table 3 provides examples of representative vector for expression of DENV-2 DI RNA. These are also shown pictorially in FIG. 2 (I and J).

TABLE 3
Example vectors for DI RNA expression

| Name | Description |
|---|---|
| pCDH-CMVp-DENV-2 DI_290- | Dual promoter lentivector using a CMV |
| HDVr-Poly A-PGKp-CFP-T2A- | promoter to express DENV-2 DI 290 |
| puromycin | RNA and hepatitis delta virus ribozyme |
| | (HDVr) sequence. |
| pCDH-CMVp-DENV-2 DI_290- | Dual promoter lentivector using a CMV |
| HDVr-S/MAR-Poly A-PGKp- | promoter to express DENV-2 DI 290 |
| GFP-T2A-puromycin | RNA, HDVr and Scaffold/matrix |
| | attachment region (S/MAR) sequence. |

Table 4 shows examples of vector, promoter and genetic element combinations for the expression of DENV non-structural proteins or DI RNA.

TABLE 4
Representative vectors

| | | | | ORF | |
|---|---|---|---|---|---|
| | Promoter | | Promoter | Fluorescent | Genetic |
| Vector | 1 | ORF | 2 | protein | element |
| pCDH | EF1α | DENV-2 | PGK | EGFP or | ±S/MAR |
| | (short | (monkey codon | | mCherry | |
| | promoter) | optimised | | | |
| | | DENV-2 | | | |
| | | NS1-NS5 orf) | | | |
| pCDH | EF1α | DENV-2 | PGK | EGFP or | ±S/MAR |
| | (short | (human codon | | mCherry | |
| | promoter) | optimised | | | |
| | | DENV-2 | | | |
| | | NS1-NS5 orf) | | | |
| pCDH | EF1α | DENV-2 | PGK | EGFP or | ±S/MAR |
| | (Full | (human codon | | mCherry | |
| | promoter) | optimised | | | |
| | | DENV-2 | | | |
| | | NS1-NS5 orf) | | | |
| pCDH | EF1α | DENV-2 | PGK | EGFP or | ±S/MAR |
| | (Full | (monkey codon | | mCherry | |
| | promoter) | optimised | | | |
| | | DENV-2 | | | |
| | | NS1-NS5 orf) | | | |
| pCDH | CMV | Defective | PGK | CFP | ±S/MAR |
| | | Interfering | | | |
| | | RNA | | | |

Table 5 shows examples of vector, promoter and genetic element combinations for the expression of DENV structural proteins.

TABLE 5
Representative vectors

| | Promoter | | Promoter | Genetic | | Genetic |
|---|---|---|---|---|---|---|
| Vector | 1 | ORF | 2 | element | ORF | element |
| pSRS11 | EF1α | DENV-2 (human codon | | IRES | EGFP or | ±S/MAR |
| | (Full | optimised DENV-2 | | | mCherry | |
| | promoter) | CprME orf) | | | | |
| pSRS11 | EF1α | DENV-2 (human codon | | IRES | EGFP or | ±S/MAR |
| | (short | optimised DENV-2 | | | mCherry | |
| | promoter) | CprME orf) | | | | |
| pCDH | EF1α | DENV-2 (human codon | PGK | | EGFP or | ±S/MAR |
| | (Full | optimised DENV-2 | | | mCherry | |
| | promoter) | CprME orf) | | | | |
| pCDH | EF1α | DENV-2 (human codon | PGK | | EGFP or | ±S/MAR |
| | (short | optimised DENV-2 | | | mCherry | |
| | promoter) | CprME orf) | | | | |

### Cell Culture

The skilled person would understand that the cell lines of the disclosure can be derived from any cell which can be cultured in vitro and in which a Flaviviridae can replicate. The preferred cell line is derived from a Flaviviridae host or carrier.

In one example, the cell line is of mammalian, avian or arthropod origin. In one example, the cell line is mammalian. In one example, the cell line is human (e.g. HEK 293T). In one example, the cell line is derived from a primate cell (e.g. a Vero cell). In one example, the cell line is derived from a livestock cell. In one example, the cell line is avian. In one example, the cell line is derived from an arthropod cell. In one example, the arthropod is a mosquito or a tick. In one example, the cell line is a continuous cell line. In one example, the cell line is a primary cell line. In one example, the cell line is an immortalized cell line. In one example, the cell line is adherent. In one example, the cell line in a non-adherent (suspension cell). In one example, the cell line is vaccine certified.

The cell line of the disclosure can be cultured in any cell culture medium that allows the expansion of the cells in vitro and allows for expression and production of DIPs. Exemplary cell culture mediums for culturing the cell of the present invention include, but are not limited to: Iscove's medium, UltraCHO, CD Hybridoma serum free medium, episerf medium, MediV SF103 (serum free medium), Dulbecco's modified eagle medium (DMEM), Eagles Modified Eagle Medium (EMEM), Glasgow's modified eagle medium (GMEM), SMIP-8, modified eagle medium (MEM), VP-SFM, DMEM based SFM, DMEM/F12, DMEM/Ham's F12, VPSFM/William's medium E, ExCell 525(SFM), adenovirus expression medium (AEM), Excell 65629 and Happy Cell® Advanced Suspension Medium. In a preferred embodiment, the cell line is cultured in Happy Cell® Advanced Suspension Medium.

It will be appreciated by persons skilled in the art that such mediums may be supplemented with additional growth factors, for example, but not limited, amino acids, hormones, vitamins and minerals.

In one example, the cell line is cultured in stationary culture. In one example, the cell line is cultured in stirred cultured. In one example, the cell line is cultured in a bioreactor. In one example, the cell line is cultured in a wave bioreactor. In one example, the cell line is cultured in batch cell line culture. In one example, the cell line is cultured in perfusion cell line culture. In one example, the cell line is cultured in a seed medium and a production medium. In one example, the culture is from about 500 mL 1 L to about 2500 L.

In one example, the cell line as described herein is cultured at a temperature of about 37° C. to about 40° C. during DIP production. In on example, the cell line as described is cultured at a temperature of about 38° C. to about 39.5° C. during DIP production. In on example, the cell line as described is cultured at a temperature of about 38° C. to about 39.5° C. during DIP production. In one example, the cell line as described herein is cultured at a temperature of about 39° C. during DIP production.

### Methods of Harvesting DIPs

The disclosure provides methods of harvesting a cloned or recombinant virus DIP expressed by the cell line as described herein, or produced by the method described herein.

For example, harvesting DIPs can involve one or more of the following steps: clarification, concentration, DNA/RNA removal, separation/purification, polishing and sterile filtration (Wolf et al., 2008; Wolf et al., 2011; Kalbfuss et al., 2006; Josefsberg et al., 2012). In one example, clarification is performed by centrifugation, microfiltration and/or depth filtration. In one example, concentration is performed by centrifugation, ultrafiltration, precipitation, monoliths and/or membrane adsorber. In one example, DNA/RNA removal is performed by nuclease treatment. In one example, the nuclease treatment is treatment with benzonase. In one example, separation/purification is performed by ultracentrifugation (for example density gradient), bead chromatography (for example size exclusion chromatography, ion exchange chromatography or affinity chromatography), hydroxyapatite chromatography and/or membrane adsorber (for example ion exchange chromatography or affinity chromatography). In one example, polishing is performed by ultrafiltration and/or diafiltration. In one example, DIPs can be concentrated by alcohol or polyethylene glycol precipitation.

In one example, the method of harvesting a DIP from cell and/or culture comprises: i) clarification; ii) nuclease treatment; (iii) reducing cell debris; and iv) purification. In one example, the method of harvesting a DIP from cell and/or cell culture comprises: i) centrifugation; ii) benzonase nuclease treatment; iii) filtration; iv) and hydroxyapatite chromatography.

In one example, the DIP can be harvested from the cell culture medium. In one example, the cell as described herein may be lysed and DIPs additionally collected from the lysed cells.

As used herein “Isolated” is substantially or essentially free from components that normally accompany it in its native state. For example, an isolated DIP is substantially or essentially free from cellular debris and cell culture medium.

### Compositions

In an aspect, the disclosure provides a pharmaceutical composition comprising the DIP as described herein.

The term “pharmaceutical composition”, as used herein, means any composition, which contains at least one therapeutically or biologically active agent and is suitable for administration to the patient. The pharmaceutical composition may comprise one or more pharmaceutically acceptable excipients. Any of these formulations can be prepared by well-known and accepted methods of the art. See, for example, Gennaro, A. R., ed., Remington: The Science and Practice of Pharmacy, 20th Edition, Mack Publishing Co., Easton, Pa. (2000).

The phrase “pharmaceutically acceptable” is employed herein to refer to those compounds, materials, compositions, and/or dosage forms that are, within the scope of sound medical judgment, suitable for use in contact with the tissues of human beings and animals without excessive toxicity, irritation, allergic response, and/or other problem or complication, commensurate with a reasonable benefit/risk ratio.

In an aspect, the disclosure provides an immunogenic composition comprising a DIP as described herein. As used herein, “immunogenic composition” refers to a substance (e.g. DIP as described herein) which is able to provoke an immune response in the body of a human or other animal. In one example the immunogenic composition is a vaccine. The term “vaccine” as used herein refers to a composition comprising at least one immunologically active component that induces an immunological response in a subject and possibly but not necessarily one or more additional components that enhance the immunological activity of said active component (for example an adjuvant). A vaccine may additionally comprise further components typical to pharmaceutical compositions. The vaccine may be an RNA or protein vaccine. In one example, the vaccine composition produced is suitable for human use. In one example, the vaccine composition produced is suitable for veterinary use.

In one example, the DIP is in vector form. In another example, the DIP is a nucleic acid sequence comprising or consisting of the sequence of SEQ ID NO:26, or SEQ ID NO:27.

In one example, the immunogenic composition or vaccine further comprises an adjuvant. Illustrative adjuvants include: particulate or non-particulate adjuvants, complete Freund's adjuvant (CFA), aluminium salts, emulsions, ISCOMS, LPS derivatives such as MPL and derivatives thereof such as 3D, mycobacterial derived proteins such as mural di- or tri-peptides, particular sapiens from Quill Aja sayonara, such as QS21 and ISCOPREP™ spooning, ISCOMATRIX™ adjuvant, and peptides, such as thyroxin alpha 1. An extensive description of adjuvants can be found in Cox and Coulter, “Advances in Adjuvant Technology and Application”, in Animal Parasite Control Utilizing Biotechnology, Chapter 4, Ed. Young, W. K., CRC Press 1992, and in Cox and Coulter, Vaccine 15(3): 248-256, 1997.

In an aspect, the disclosure provides a method of reducing the load of a viral RNA in a subject comprising administering to the subject the DIP as described herein, the pharmaceutical composition as described herein or the immunogenic composition as described herein.

In an aspect, the disclosure provides a method of reducing the load of a Flavivirus RNA in a subject comprising administering to the subject the DIP as described herein, the pharmaceutical composition as described herein or the immunogenic composition as described herein.

In one example, the load of a viral RNA is reduced about 10% to about 90%, about 25% to about 75%, about 30% to about 60%, or about 50% compared to the viral load before administration of the DIP to the subject. In another example, the viral load is reduced at least 80%, at least 70%, at least 60%, or at least 50% compared to the viral load before administration of the DIP to the subject.

The compositions may additionally comprise a preservative, a buffering agent, or a stabilizing agent.

The compositions as described herein can be administered to a subject/host by a parenteral or non-parenteral route of administration. Parenteral administration includes any route of administration that is not through the alimentary canal (that is, not enteral), including administration by injection, infusion and the like. Administration by injection includes, by way of example, into a vein (intravenous), an artery (intra-arterial), a muscle (intramuscular) and under the skin (subcutaneous). The composition as described herein may also be administered in a depot or slow release formulation, for example, subcutaneously, intradermal or intramuscularly, in a dosage which is sufficient to obtain the desired pharmacological effect.

### Reducing Viral Transmission

The DIP/DIPs as described herein can be used to reduce the transmission of a virus, for example a Flaviviridae, between a viral host and a viral carrier. Accordingly, they can be used to prevent, reduce and manage the severity of viral outbreaks.

In an aspect, the disclosure provides a method of reducing transmission of a virus between a viral host and a viral carrier comprising administering the DIP as described herein, the pharmaceutical composition as described herein or the immunogenic composition as described herein to the viral host.

In an aspect, the disclosure provides a method of reducing transmission of a Flaviviridae between a Flaviviridae host and a Flaviviridae carrier comprising administering the DIP as described herein the pharmaceutical composition as described herein or the immunogenic composition as described herein to the Flaviviridae host. The DIP may be administered to the host in any of the above described manners.

In an aspect, the disclosure provides a method of reducing the risk of a viral outbreak or severity of a viral outbreak in a population of hosts comprising administering to a plurality of hosts in the population the DIP as described herein, the pharmaceutical composition as described herein or the immunogenic composition as described herein.

In one example, the composition described herein is administered to the host in one or more of the following conditions: i) before the host is infected with a virus/Flaviviridae; ii) if a host has been in contact with an individual infected with a Flaviviridae or in contact with a Flaviviridae; iii) after a host is infected with a Flaviviridae.

The pharmaceutical composition as described herein or the immunogenic composition as described herein may be administered in a single dose or in multiple doses.

Administering the compositions as described herein to a host before or after infection with a virus can reduce the viral load in a subject via viral interference, reducing the risk of transmission to another host or a carrier. Additionally, carriers that feed on the host administered DIP may acquire the DIP from the host reducing the viral load in a carrier via viral interference, consequently reducing the risk of transmission to another carrier or host.

In an aspect, the disclosure provides a method of reducing transmission of a virus between a viral host and a viral carrier comprising administering a composition as described herein to the viral carrier.

In an aspect, the disclosure provides a method of reducing transmission of a Flaviviridae between a Flaviviridae host and a Flaviviridae carrier comprising administering a composition as described herein to the Flaviviridae carrier. In one example, the composition is administered to the carrier in one or more of the following manners: i) feeding (e.g. with a bait comprising the DIP); ii) exposure to an aerosol; and iii) direct injection. In one example, the composition as described herein is administered to a carrier via feeding, aerosol or injection and the carrier comprising the DIP is released into wild/natural populations of the carrier. The DIP can propagate in the wild/natural populations of the carrier reducing the viral load in wild/natural populations via viral interference.

The present invention is not to be limited in scope by the specific examples described herein, which are intended for the purpose of exemplification only. Functionally-equivalent products, compositions and methods are clearly within the scope of the invention, as described herein.

### EXAMPLES

### Materials and Methods

### Plasmid Constructs

The lentiviral vector pCDH-EF1α-MCS-BGH-PGK-GFP-T2A-Puro was a gift from Stacey Edward, QIMR Berghofer Medical Research Institute, Australia and pCMVΔR8.91 was a gift from Andreas Suhribier, QIMR Berghofer Medical Research Institute, Australia. pCMV-VSV-G was obtained from Ian Mackay, The University of Queensland, Australia. The pCFP-coilin plasmid was a gift from Miroslav Dundr from Rosalind Franklin University, USA. The pcDNA3.MLV.GP (MLV Gag-Pol) and pSRS11-SF-γC-EGFP were gifts from Axel Schambach, Hannover Medical School, Germany.

pSicoRE11-EF1α-mCherry-T2A-DENV-2_CprME. DENV2 CpreME sequence was amplified from human codon optimized sequence using primers of forward: D2-C-opt-T2A-Xma1-For: GTC GAG GAG AAT CCC GGC CCTATGAACAACCAGCGGAAGAAG, and reverse primer D2-E-opti-Ecor1-T2A-Rev TCCCTCGACGAATTCTCAAGCCTGA ACCATC. The vector pSicoRE11-EF1α-mCherry-T2A was cut with XmaI and EcoRI and ligased with cpreME (capsid (c), premembrane (prM)/membrane (M), and envelope (E)) fragment containing structural proteins.

pCDH-EF1α-DENV-2_NS1˜NS5-BGH-PGK-GFP-T2A-Puro. DENV2 NS1-5 sequence was amplified using CloneAmp HiFi premix polymerase (Clontech) by using a DENV2 infectious clone as template and the forward and reverse primers are “D2-NS1-Ecor1-Forw”

CTAGAGCTAGCGAATTCGCCATGGCACCTCACTGTCTGTGTCATT and “D2-NS5-BamH1-Rev” ACAGTCGGCGGCCGCGGATCCCTACCACAAGACTCCTGCCT. The pCDH-EF1α-BGH-PGK-GFP-T2A-Puro vector is cut with BamH1 and EcoR1 and inserted NS1-5 fragment by in-fusion. Same strategy was used to insert monkey codon optimised DENV2 NS1-5 sequence into pCDH-EF1α-BGH-PGK-GFP-T2A-Puro vector and the vector with full-length EF1α promoter.

To create the pCDH-EF1α-MCS-BGH-PGK-CFP-T2A-Puro plasmid, Cyan fluorescent protein (CFP) was PCR amplified from pCFP-coilin (forward primer 5′-AAAAACCTAGGATGGTGAGCAAGGGCGAG and reverse primer: 5′-TTTTTATGCATCTTGTACAGCTCGTCCATGC) and pCDH-EF1α-MCS-BGH-PGK-T2A-Puro was inverse PCR amplified from the pCDH-EF1α-MCS-BGH-PGK-CFP-T2A-Puro plasmid (forward primer 5′-AAAAAATGCATGAGGGCAGAGGAAGTCTTCT and reverse primer 5′-TTTTTCCTAGGCGGTCTCTGCTGCCTCAC). The CFP gene was then inserted into the pCDH-EF1α-MCS-BGH-PGK-T2A-Puro using In-Fusion cloning kit (Clontech) according to the manufacturers' instructions. pCDH-CMV-DENV-2_DI 290-HDVr-BGH-PGK-CFP-T2A-Puro was generated by inserting CMV-DENV-2_DI 290-HDVr (synthesised from GenScript) into the pCDH-EF1α-MCS-BGH-PGK-CFP-T2A-Puro via Hpa I and Not I restriction sites.

The retroviral and lentiviral vectors are self-inactivating vectors comprising a deletion of the U3 region in the long terminal repeat sequences.

### Cell Lines and Virus-Like Particle (VLP) Production

HEK293T, Vero E6 (also referred to as “Vero” cells in this document) and Phoenix-Ampho cells were cultured in Dulbecco's Modified Eagle medium (DMEM) (Life Technologies) supplemented with 10% (v/v) fetal bovine serum (Life Technologies) and 1% (v/v) penicillin-streptomycin. All cells were incubated at 37° C. in a humidified 5% CO2 atmosphere.

To generate lentiviral particles containing DENV-2 NS1˜NS5 gene, DENV-2 CpreME or DENV-2_DI 290 gene, HEK293T cells were cultured in 10-cm dishes and co-transfected with 6 μg of pCMVΔR8.91 plasmid, 2 μg of pCMV-VSV-G and 2 μg of pCDH-EF1α-DENV-2_NS1˜NS5-BGH-PGK-GFP-T2A-Puro, pSicoRE11-EF1α-mCherry-T2A-DENV-2_CprME or pCDH-CMV-DENV-2_DI 290-HDVr-BGH-PGK-CFP-T2A-Puro using X-tremeGENE HP DNA transfection reagent (Roche) according to the manufacturers' instructions. VLPs containing DENV-2 CprME gene were produced in Phoenix-amphotropic retroviral packaging producer cell line by co-transfection of 10 μg of pSRS11-EF1α-mCherry-T2A-DENV-2_CprME and 2 μg of pcDNA3.MLV.GP (MLV Gag-Pol expressing plasmid) using X-tremeGENE HP DNA transfection reagent (Roche) according to the manufacturers' instructions in a 10-cm dish. At 48 h post-transfection, cell culture supernatants containing VLPs were harvested, filtered through 0.45 μm filters and stored in small aliquots at −80° C. until needed.

### Cell Growth in Happy Cell Advanced Suspension Medium

Happy Cell Advanced Suspension Medium (ASM, 4×) was obtained from Vale Life Sciences. Briefly, HEK293T cells producing DIP D2-290nt were seeded at 4×10⁵ or 8×10⁵ (as recommended by the manufacturer) in 2 ml of culture medium supplement with ASM at a final concentration of ASM of 1×, 2× or 3×. The cells were incubated at 39° C. for 3 days. The cell density was measured by counting using trypan blue staining and a haemocytometer. DIP were quantified by centrifugation of DIPs at 100,000×g for 1 h and then measuring the level of DI RNA_D2-290nt by RT-qPCR.

### Generation of Cell Lines Stably Expressing DENV-2 Viral Proteins and DENV-2 DI 290 RNA

HEK293T and Vero cells were transduced with lentivirus and retrovirus prepared as described above. Cells were transfected as described in Jin et al., (2016); MBio. 5; 7(4); Apolloni et al., (2013) Hum Gene Ther. 24(3):270-82; and Lin et al., (2014) 14; 11:121. After 24 h transduction, cells were washed, replaced with new culture medium and further incubated for 48 h. At 72 h post-transduction, cells either were purified by FACS or selection by puromycin. For FACS, the cells were trypsinised, filtered through 37 pm Nylon Mesh to remove cellular clumps and diluted in PBS to a concentration of 2×10⁷ cells/ml or selected by puromycin. Fluorescence-activated cell sorting (FACS) analyses were performed to isolate cells expressing high levels of GFP, mCherry and CFP using a FACS ARIA III cell sorter (BD Biosciences).

### Immunofluorescence Analysis

Cells were grown on glass coverslips, fixed in 4% (w/v) paraformaldehyde at room temperature for 10 min and quenched with 50 mM NH4Cl for 5 min. Cells were then permeabilised with 0.1% (v/v) Triton X-100 for 15 min and blocked in 10% (v/v) normal goat serum (Sigma Aldrich) for 15 min. DENV-2 NS3 protein was detected with a rabbit anti-DENV NS3 polyclonal antibody (Sigma Aldrich). DENV-2 E and CA were probed with a rabbit anti-DENV E polyclonal antibody (GeneTex) and a rabbit anti-DENV CA polyclonal antibody (Novusbio), respectively. dsRNA was probed with a mouse anti-dsRNA monoclonal antibody J2 (SCICONS). Primary antibodies were detected with Alexa Fluor 647-conjugated goat anti-rabbit antibodies (Thermo Fisher Scientific) or Cy5-conjugated goat anti-mouse antibodies (Life Technologies). Nuclei were stained with 1 μM 4′,6-diamidino-2-phenylindole (DAPI) (Life Technologies). Finally, coverslips were mounted onto slides with ProLong Gold antifade reagent (Life Technologies). Fluorescent images were captured using a Zeiss 780 NLO confocal scanning microscope (Zeiss) with 63× objective lenses and standard lasers and filters for Alexa Fluor 647, Cy5 and DAPI fluorescence.

### Western Blot Analysis

Cell were lysed at 4° C. for 30 min with lysis buffer (50 mM Tris-HCl, pH 7.4; 150 mM NaCl; 1 mM EDTA; 1% [v/v] Triton X-100; protease inhibitor cocktail [Roche]). Cell lysates were centrifuged at 12,000×g for 10 min and clarified supernatants were collected. The total protein concentrations were determined by the Bradford method against a bovine serum albumin standard. 30 μg of cell lysates were boiled in dodecylsulfate—polyacrylamide gel electrophoresis (SDS-PAGE) sample buffer (125 mM Tris-HCl, pH 6.8; 4% [v/v] SDS; 20% [v/v] glycerol; 0.004% [w/v] bromphenol blue) and separated by 10% SDS-PAGE. Gels were electro-blotted onto a polyvinylidene fluoride (PVDF) membrane (Pall) using a semi-dry transfer system (Bio-Rad Laboratories). DENV-2 E and CA proteins were detected with a rabbit anti-DENV E polyclonal antibody (GeneTex) and a rabbit anti-DENV CA polyclonal antibody (Novusbio), respectively. DENV-2 NS3 and DENV-2 NS5 were detected with a rabbit anti-DENV NS3 polyclonal antibody (Sigma Aldrich) and a mouse anti-DENV NS5 monoclonal antibody (GeneTex). dsRNA was detected with a mouse anti-dsRNA monoclonal antibody J2 (SCICONS). Endogenous β-tubulin was detected with a mouse anti-β-tubulin monoclonal antibody (Sigma Aldrich). Primary antibodies were detected with anti-rabbit IgG horseradish peroxidase (HRP)-linked antibodies or anti-mouse IgG HRP-linked antibodies (Cell Signalling Technology).

### RNA Extraction and RT-qPCR Assay

Total RNA from cells was isolated with TRIzol reagent (Thermo Fisher Scientific, Waltham, Mass.) in accordance with the manufacturer's protocol. For culture supernatant RNA extraction, supernatants were centrifuged at 1,000×g for 10 min and passed through a 0.45 μm filter. Then, the clarified supernatants were pelleted by ultra-centrifugation at 100,000×g for 1 h at 4° C. The RNA from the pelleted material was isolated with TRIzol reagent according to the manufacturers' instructions. All RNA samples were treated with Turbo DNase I (Thermo Fisher Scientific). cDNA was made using random hexamer primers (New England Biolabs) and Superscript IV reverse transcriptase (Thermo Fisher Scientific) according to the manufacturers' instructions. DENV RNA was quantified by using oligonucleotide primers that targeted the human codon optimised E gene (forward primer 5′-ACCAGGTGTTCGGCGCC and reverse primer 5′-TTCAAGCCTGAACCATCACGC), monkey codon optimised NS1 gene (forward primer 5′-GAGACCTCAGCCTACCGAGCT and reverse primer 5′-TTGGAGTCGCAAGACACGTC), NS5 gene (forward primer 5′-GCCTGATGTACTTCCACAGA and reverse primer 5′-ATTGCCTATTAGGGATCTAAC) and monkey codon optimised NS5 (forward primer 5′-TGGTCTATCCATGCCACCCAT and reverse primer 5′-ATGTAGTCGGTGTACTCCTCA) regions. DENV DI RNA was quantified using oligonucleotide primers: forward primer 5′-GAGAGAAACCGCGTGTCGAC and reverse primer 5′-AGAACCTGTTGATTCAACAG. For cell samples, DENV-2 RNA and DI RNA copy numbers were normalised to the level of GAPDH mRNA using oligonucleotide primers: forward primer 5′-GCAAATTCCATGGCACCGTC and reverse primer 5′-TCGCCCCACTTGATTTTGG. SYBR green master mix (Bio-rad Laboratories) was used for qPCR according to the manufacturers' instructions.

### DIP Purification

Supernatant (200 ml) from DIP producing cells is purified by column chromatography and concentrated by centrifugal filtration.

### Velocity Gradient Analysis

One ml of DIP containing supernatants were loaded onto a 10 ml 5-50% w/v sucrose/PBS (pH 7.4) gradient and centrifuged at 80,000×g for 2.5 h at 4° C. in a SW40i rotor (Beckman). Fractions (1 ml) were collected from the gradient by puncturing the bottom of the centrifuge tube. RNA from 100 μl of fractionated samples was extracted and assayed for DI RNA copy number by RT-qPCR.

### Dot Blotting

200 μl of fractionated samples were added to nitrocellulose membrane (Amersham Biosciences). The membrane then was blocked in 5% w/v skim milk in PBST for 1 h at room temperature before being incubated in anti-flavivirus E or anti-capsid antibodies (4G2, GF3.1, the gifts from John Aaskov, Queensland University of Technology, Australia) overnight at 4° C. Primary antibodies were detected with anti-mouse IgG HRP-linked antibodies (Cell Signalling Technology).

### CHT Ceramic Hydroxyapatite Chromatography

A column (15 mm×100 mm, Bio-rad Laboratories) was packed with 40-μm CHT™ ceramic hydroxyapatite Type II Media (Bio-rad Laboratories) and set on a US MFLEX Easy-Load system (MasterFlex). The flow rate was 1 ml/min. The packed column was rinsed with 600 mM sodium phosphate buffer (NaPB) pH 7.2 and equilibrated with 10 mM NaPB pH 7.2. Culture supernatants were then loaded onto the column, wash with 10 mM NaPB pH 7.2 and eluted with 350 mM NaPB pH 7.2.

### Centrifugal Filtration

The CHT ceramic hydroxyapatite-elution containing DIPs were filtered and exchange buffer to PBS using an Amicon Ultra Centrifugal Filter with a 100K Da cut-off (Merck) by centrifugation at 4,000×g for 25 min at 4° C. The concentrate was stored in small aliquots at −80° C. 1 ml of filtrate and 50 μl of concentrate were collected and subjected to the analysis of DI RNA levels by RT-qPCR.

### Analysis of Dengue Virus Defective Interfering Particle (DENV-2 DIP) Antiviral Activity

Vero E6 or Huh7 cells were seeded in 12-well plates at a density of 100,000 cells/well. On the next day, the cells were infected with DENV-1˜4 at MOI 0.1 or 1. At 3 h post-infection, the cells were washed twice with PBS and replaced with fresh 1 ml of culture medium containing DENV DIPs at the concentration indicated. After 2 and 5 days of incubation, 100 μl of culture supernatants were collected and the concentration of DENV-2 genomic RNA was measured by RT-qPCR using primers to DENV1˜4 NS5 gene or a viral titre was measured by plaque assay.

### Plaque Assay

Vero cells were seeded in 96-well plates at 3×10⁴ cells per well and incubated overnight at 37° C. with 5% CO₂. Cells were inoculated with dilutions of samples for 2 h then cells were overlayed 2% high viscosity carboxymethyl cellulose (CMC) in Medium 199 (Sigma-Aldrich, C5013) and supplemented with 2% v/v FBS (Life Technologies). Cells were incubated at 37° C. with 5% carbon dioxide for 6 days before fixation with 1:1 v/v ice-cold acetone and methanol for 10 min at room temperature. Cells were blocked with LI-COR Odyssey blocking buffer (LI-COR Biosciences) for 1 h at 37° C. then incubated with mAb 4G2 (mouse anti-flavivirus envelope). Cells were washed 3 times with 0.05% PBS-T then incubated with IRDye® 800CW Goat anti-Mouse IgG (Li-Cor) for 1 h at 37° C. Cells were then washed 5 times with 0.05% PBS-T and plates were imaged at 800 nm using the Li-Cor Odyssey imaging platform (LI-COR Biosciences) to detect virus foci.

### Example 1 A DIP Stable Production System

The present inventors have developed a system to mass produce Dengue virus (DENV)-based DIPs that are free of infectious DENV. The optimised DENV open reading frames have been found to work better than natural sequences in the current system.

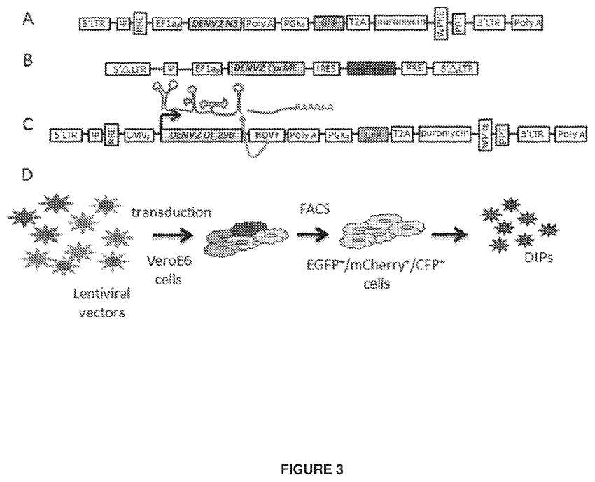

The DIP production system of this disclosure uses a Vero cell line that stably expresses DENV serotype 2 (DENV-2) structural (S) and non-structural (NS) proteins, which were introduced into cells using a lentivector and a retrovector, respectively (FIG. 3A and 3B). The DENV S and NS proteins are encoded by two separate non-overlapping codon-optimised mRNAs, so that the probability of forming recombinant virus is low and infectious DENV RNA cannot be made.

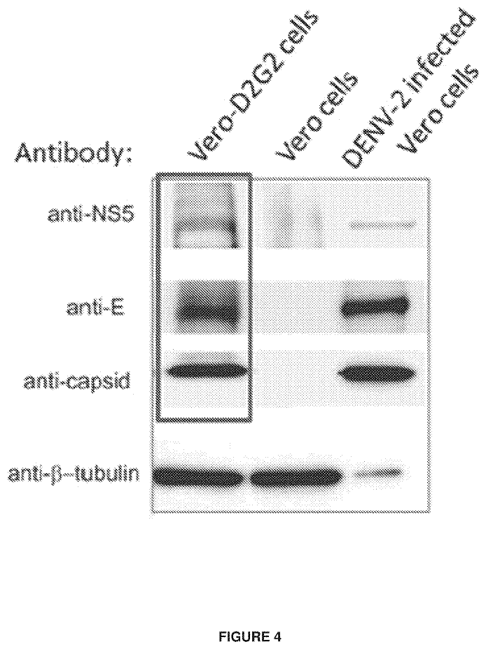

The DENV-2 NS and S proteins are stably expressed by the Vero-DENV-2-Generation-2 (Vero-D2G2) cell line (FIG. 4). The cell line, (referred to herein as the Vero-DENV-2-Generation2 (Vero-D2-Gen2) a Dengue virus serotype 2 and being of the second generation-nomenclature used by the present inventors), replicates DENV DI RNAs transfected into cells and packages the DI RNA into DIPs, which are secreted into culture supernatant. The antiviral DI RNAs thus produced are naturally occurring (meaning that they are derived from RNAs isolated from the serum of infected patients) and contain only about 3-10% of the viral genomic sequence (wherein a portion of the genomic sequence has been naturally deleted), and include all the genomic elements required for replication and packaging by DENV NS and S proteins.

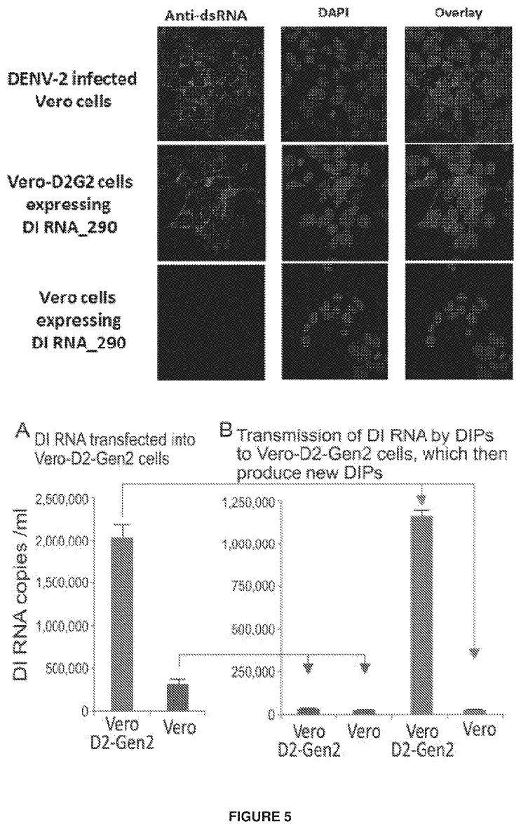

cDNA sequence corresponding to DI RNAs which are 443 and 290 nucleotides long were introduced into Vero-D2G2 cells using another lentiviral vector that makes authentic DENV DI RNA (FIG. 3C). The DI RNA replicate in Vero-D2G2 cells using the DENV RNA replicase complex (FIG. 5A), and are packaged into DIPs that are then secreted into culture supernatant.

Vero-D2-Gen2 cells transfected with a cDNA encoding DENV-2 290 nucleotide (nt) long DI RNA, referred to as D2-290nt, secrete high levels of DIPs into culture supernatant (FIG. 5B). Laboratory scale stationary cultures produce DIPs supernatant that contain up to ˜1×10⁷ DI RNA copies/ml. Cell-free DIPs in supernatant can bind to and enter parental Vero-D2-Gen2 and Vero cells, but new DIPs can only be reproduced by Vero-D2-Gen2 cells (containing the viral structural and non-structural proteins), not by unmodified Vero cells (FIG. 5C), confirming that the DIPs are transmissible. DIPs can be pelleted by ultracentrifugation (at 100,000×g), and western blots show that DIPs contain capsid and envelope proteins as expected.

The DIPs are biologically active as they can inhibit DENV replication in in vitro cell culture experiments. In preliminary experiments, DENV-2-derived DIPs reduced DENV replication by up to 1117-fold in Huh7 cells and cross-serotype inhibition was observed (Table 6). DIPs have been purified and concentrated to ˜1×10¹⁰ DI RNA copies/ml.

TABLE 6
Fold inhibition of DENV replication by DIPs
with DI RNA_290

| DENV | Fold inhibition by |
|---|---|
| Serotype | DIP with DI RNA_290 |
| 1 | 64.1 ± 9.1 |
| 2 | 44.6 ± 11.7 |
| 3 | 117.8 ± 17.2 |
| 4 | 8.3 ± 2.4 |

Huh7 cells were infected with DENV serotypes indicated (MOI 0.1) for 2 h. The virus was removed and DIPs were added (˜1 DI RNA copies per cell). The cells were grown for 4 days and the supernatant was collected. DENV genomic RNA in the supernatant was measured by RT-qPCR with oligonucleotide primers specific for the DENV NS5 open reading frame.

DIPs were also produced by transfection of Vero-D2-Gen2 cells with a DENV serotype 1 (DENV-1)-derived cDNA encoding DI RNA (called D1-443nt), demonstrating that this DENV-2 based cell line supports both DENV-2 and DENV-1 DI RNA replication, packaging and DIP secretion. DIPs with D2-290nt and D1-443nt inhibit DENV-1 and DENV-2 replication in Vero cells. Vero cells were infected with DENV-1 or -2 viruses at a multiplicity of infection (MOI) of ˜0.1 for 2 h and then treated with D2-290nt or D1-443nt DIP supernatant (˜50 copies of DI RNA/cell) or a control supernatant from Vero cells. After 5 days post-infection, the DENV levels in culture supernatant were determined by RT-qPCR for DENV-1 and DENV-2 using the NS5 genes to quantitate viral genome equivalents or infectious virus particles levels by a standard virus plaque assay as described herein (not shown). Both assays showed strong inhibition of DENV replication indicating that cross-serotype inhibition by the DIPs is possible (Table 7).

TABLE 7
Fold inhibition of DENV by DIPs in Vero cells

| DIP with DI RNA: | D2-290nt | D1-443nt |
|---|---|---|
| DENV-1: | 6.2 ± 2.1 | 98.1 ± 15 |
| DENV-2: | 33.8 ± 6.3 | 5.5 ± 1.7 |

The fold inhibition of DENV-1 and DENV-2 genomic RNA levels in supernatant from DIP-treated cells compared to Neg Ctrl treated cells is shown in Table 7. No statistical difference between virus levels in supernatants from Neg Ctrl-treated and untreated cells was observed (not shown). The mean value and SD of three experiments is shown.

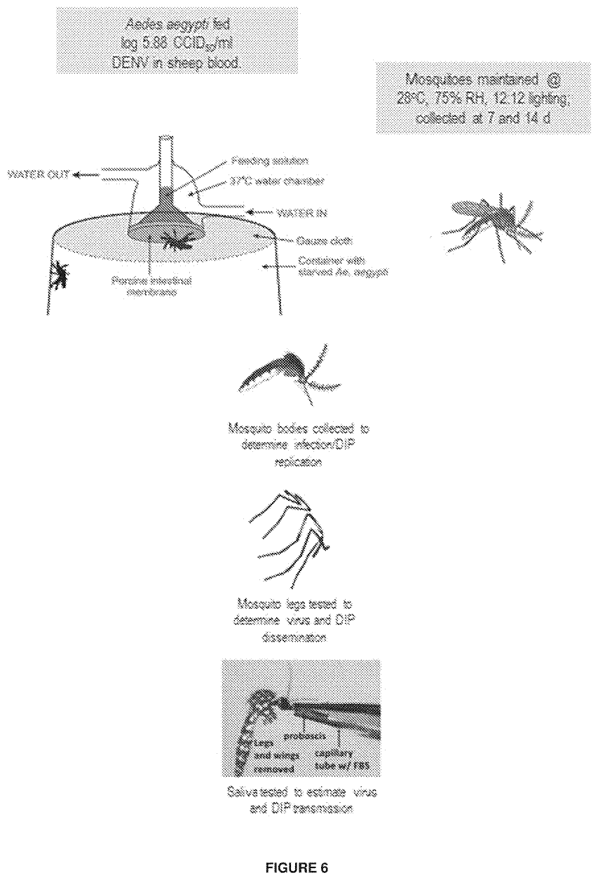

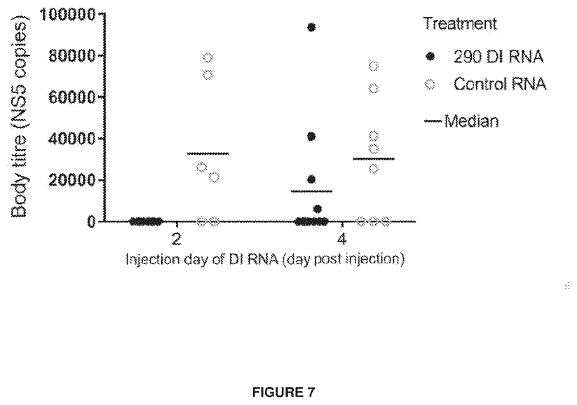

DIPs present in DENV infected blood meals fed to Aedes aegypti mosquitoes resulted in DENV infected mosquitoes that had these same DI RNAs in their bodies, legs/wings and saliva, suggesting that both virus and DIPs are able to be transmitted from vertebrate blood to mosquitoes where they may replicate. The techniques to introduce DENV and DIPs to mosquitoes using a blood meal apparatus are shown in FIG. 6. Methods to analyse DENV titre and identify DIPs in mosquitoes' bodies, wings, legs and saliva has been established. The Mosquito Control Group has microinjected DIP DI RNA into mosquito thorax that reduced DENV infection in mosquitoes (FIG. 7) and are described in Hugo et al (2016) Parasit Vectors, 9(1):555. The DIP DI RNA was in vitro purified and then micro-injected, meaning that 0.1 μl is injected into the mosquito.

### Example 2 In Vitro Testing of Potent DIPs Against DENV Serotvpes and Other Flaviviruses

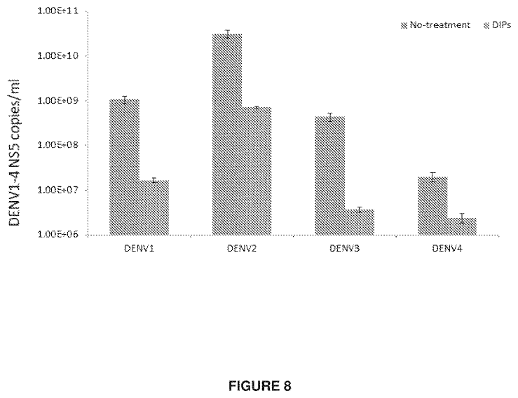

DENV derived DIP can inhibit the replication of all four DENV serotypes. As shown in FIG. 8, Huh7 cells were infected with each DENV serotype. Following removal of virus, DENV D2-290nt was added to the cells for the 72 h. It was observed that all DENV serotypes could be strongly inhibited by a single DI RNA.

### Example 3 DENV-2 DIPs can Inhibit Replication of Zika Virus (ZIKV)

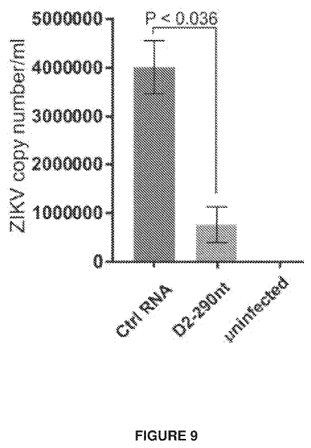

Whether DENV DI RNA can inhibit replication of other Flaviviruses such as ZIKV was examined as Flaviviruses share RNA structure homology in the 5′ and 3′ RNA UTRs that regulates virus RNA replication. To test this, the D2-290nt DI RNA or a control RNA were delivered to human HuH7 cells in triplicate and then infected with ZIKV (MOI of 0.01) for 3 h (FIG. 9). Uninfected HuH7 was included as a negative control. HuH7 cells are often used to investigate replication of ZIKV. Supernatant samples were collected after 3 days post infection and assayed for levels of ZIKV genomic RNA by RT-qPCR in triplicate. The level of ZIKV genomic RNA in supernatant from D2-290nt-treated cells was reduced by ˜5-fold compared to control-RNA-treated cells. A Student's t-test P value showed that reduced viral genome levels by D2-290nt compared to the Ctrl RNA was significant. This result shows that DENV DIPs can inhibit different members of the Flavivirus family.

### Example 4 DIPs Inhibit DENV-2 Replication in a Dose-Dependent Manner

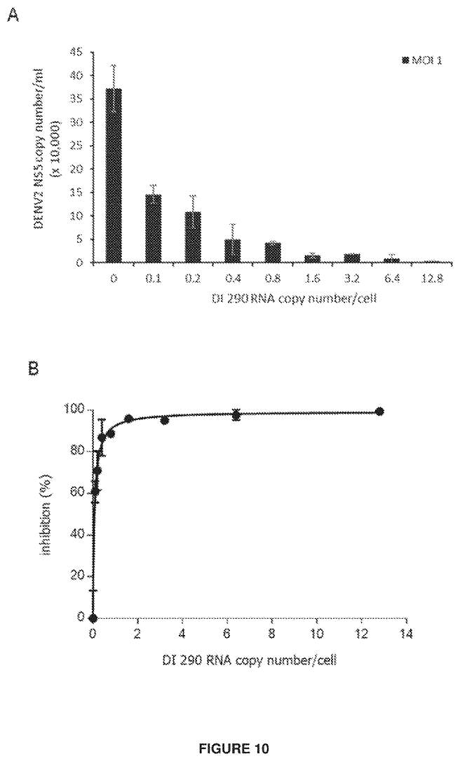

To measure the ICso of purified DIPs, 5×10⁴ Huh7 cells were infected with DENV-2 at a multiplicity of infection (MOI) of 1 for 3 hours and then cell medium was replaced with fresh culture medium containing DIPs at 0.1, 0.2, 0.4, 0.8, 1.6, 3.2, 6.4, and 12.8 DI RNA290 copies/cell. At 48 h post-infection, the RNAs from culture supernatants were extracted and the concentration of DENV-2 genomic RNA in culture supernatants were measured by RT-qPCR using oligonucleotide primers to DENV NS5 gene (FIG. 10A). The ICso was calculated using graph pad prism8 (FIG. 10B).

### Example 5 Episomal Long Term Expression of DI RNA

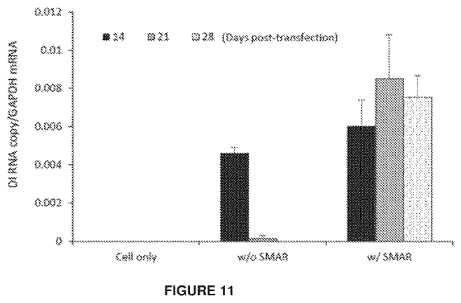

The effect of episomal long term expression of DI RNA was assessed using S/MAR. Huh 7 cells were transfected with pCDH that can produce DI-RNA 290 and also contains the scaffold/matrix attachment region (S/MAR) element. As a control, Huh 7 cells were transfected with the same pCDH plasmid that missing the S/MAR genetic element. FIG. 11 shows production of DI RNA 290 when the plasmid contains S/MAR (i.e. with (w/) S/MAR) in samples collected at 14, 21 and 28 (last time point available) days post-transduction, but not when S/MAR is omitted (i.e. without (w/o) S/MAR). Production of DI RNA 290 in samples taken at 14 days post-transduction without S/MAR was observed but was not measurable in samples collected at day 28 post-transfection. The conclusion is that S/MAR supported stable expression of DI RNA 290 in Huh 7 cells. S/MAR could support stable expression of DENV orf in any mammalian cell line and without using lentiviral or retroviral vectors that integrate into cellular chromosomes. Examples of some vectors suitable for DIP production comprising the S/MAR sequences are provided in Tables 4-5.

### Example 6 Happy Cell® Advanced Suspension Medium (ASM) Improves Cell Density and DIP Production

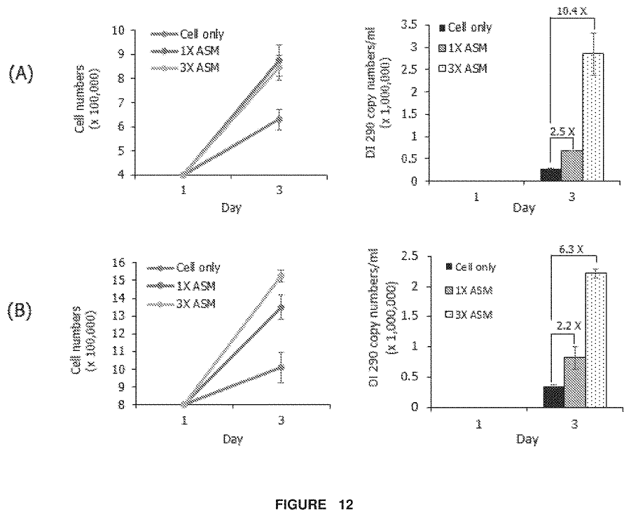

To test ASM's performance in DIP production, 4×10⁵ and 8×10⁵ HEK293T DIP-producing cells (transfected with three vectors as shown in FIG. 3) were seeded into each well of a non-treated 24 well plate with Happy cell ASM medium (stock:4×) at a final concentration of 1× and 3×ASM, or in normal medium (Cell only samples). On day 3, inactivation solution was added to disrupt the ASM suspension polymer complex and the total cell numbers were determined using a haemocytometer (FIG. 12A, left) and (FIG. 12B, left). The RNAs from the culture supernatants were extracted and subjected to RT-qPCR to measure levels of DI 290 RNA (FIG. 12A, left) and (FIG. 12B, left). Culturing in higher concentrations of ASM increases cell density and DIP production.

### Example 7 Cell Culture at Higher Temperatures Improves DIP Production

Culture of HEK 293T and HEK293T-D2-D1 producing cells was investigated at different culture temperatures. The cells were cultured in Happy Cell Advanced Suspension Medium as described. Various culture temperatures from 35° C. to 39° C. were investigated for their effects of DIP production.

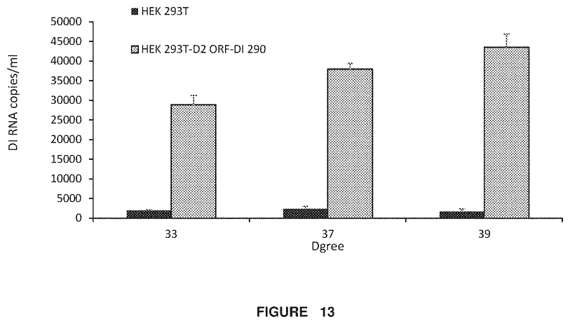

Surprisingly, and in contrast to the prior art (Ansarah-Sobrinho C et al. (2008) Virology 381:67-74), it was found that when the cells were grown at 33° C., DIP production yield was not reduced compared to when the cells were grown at 37° C. and 39° C. with continuous stirring. In fact, the yield of DIP production was about 6-10-fold higher when the cells were grown at 39° C. compared to growth at lower temperatures (FIG. 13).

### Example 8 Mosquito DENV-2 Challenge with DI RNA290 Microiniection Treatment

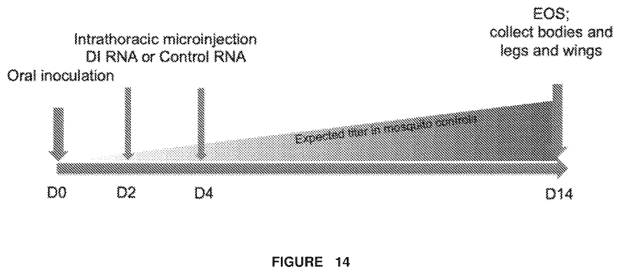

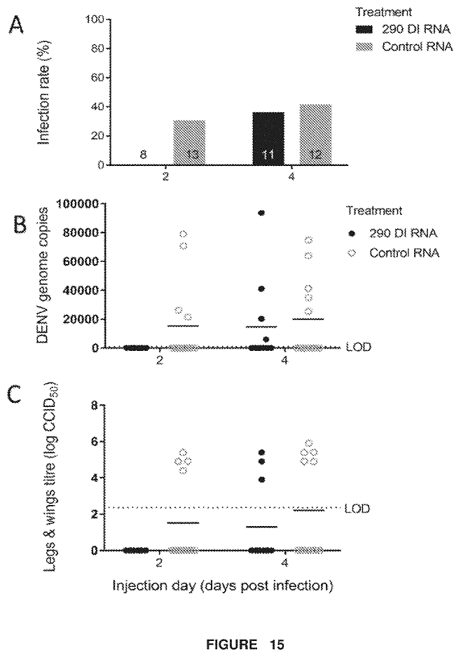

The procedure for oral infection of mosquitoes and treatment with DI RNAs by intrathoracic microinjection is shown in FIG. 14. Individual mosquitoes were fed with a blood meal containing 10⁸ CCID50/ml DENV-2 (QML16 strain) on day 0. Mosquitoes were microinjected with DENV-2 290 DI RNA or control (scrambled sequence) RNA at 2 or 4 days post infection (dpi). Mosquitoes were dissected and nucleic acid samples were collected at 14 dpi. RT-qPCR and primers to the DENV NS5 region of the viral genome were used to detect DENV-2 infection. The outcome suggests that microinjecting 290 DI RNA into mosquitoes up to two days post infection (dpi) clears virus infection in mosquito bodies by 14 days post infection (dpi) (FIG. 15).

### Example 9 Production of DIPs in HEK293 Cells

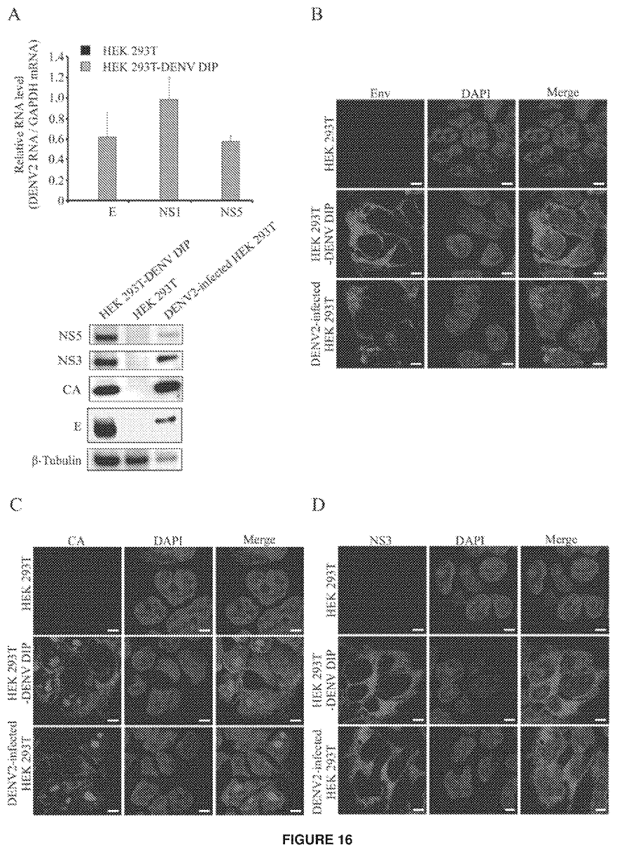

The inventors were also able to show that DENV-2 structural and non-structural proteins can be stably expressed in HEK293T cells. The expression of DENV-2 mRNA in the HEK293T DIP producing cell line was measured by RT-qPCR using oligonucleotide primers to DENV-2 E, NS1 and NS5 genes as described herein (FIG. 16A top panel). Moreover, the expression and cellular distribution of viral proteins was confirmed by Western Blot (FIG. 16A bottom panel) and immunofluorescence (FIG. 16B to D) analysis using anti-E, anti-CA, anti-NS3 and anti-NS5 antibodies.

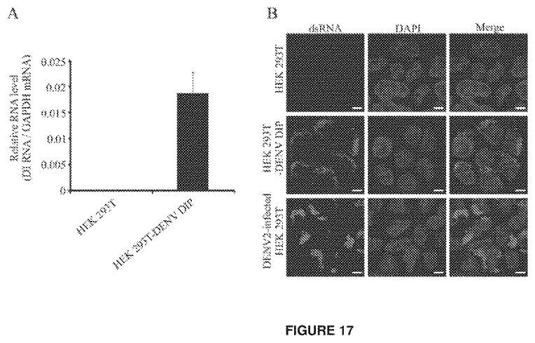

A DENV DI RNA was introduced into the cell line. Expression of the DI RNA in the cells was confirmed by RT-qPCR using primers to DI RNA. The expression of DI RNA in the cells was confirmed by RT-qPCR using primers to DI RNA (FIG. 17A). By using the antibody directed against dsRNA, dsRNA was detectable in the DIP-producing cells and DENV2-infected cells (FIG. 17B), suggesting that the stably expressed DI RNA can be replicated in cells in the presence of viral proteins. Particle production by DIP-producing cell line.

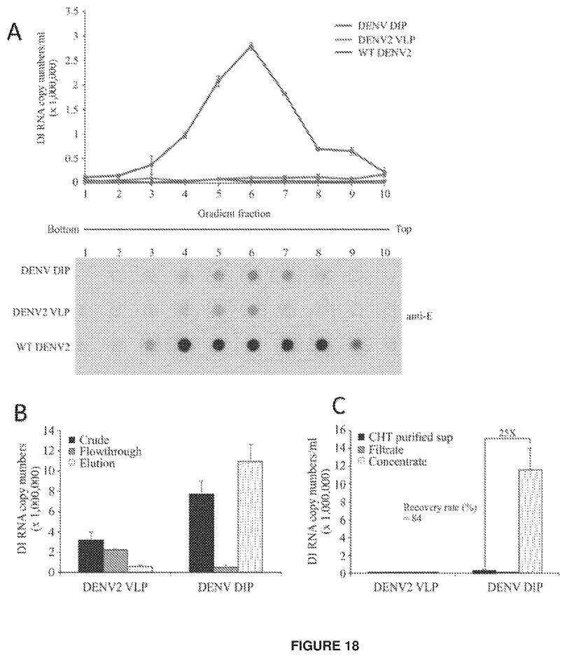

Culture supernatants from DIP-producing cells were subjected to velocity gradient (5-50% sucrose). A peak level of DI RNA and E protein was detected in gradient fraction 6 (FIG. 18A), indicating that the DIP-producing cells can secret DENV2 virus like particles into the supernatants and those virus like particles contain DI RNAs.

Culture supernatants from the DIP-producing cells were loaded onto the CHT ceramic hydroxyapatite column and eluted with sodium phosphate buffer (FIG. 18A). The CHT purified supernatants were further applied to a membrane filter device. Samples of the CHT purified supernatant, the concentrated supernatant and the flow through supernatants were ultra-centrifuged. The RNA was extracted from the pelleted material and used in RT-qPCR to measure the levels of DI RNA. The results show that DENV DIP can be purified by chromatography and concentrated membrane filtration (FIG. 18B and C).

The following examples describe a series of experiments that will be undertaken for further assess antiviral activity in vitro and in vivo in a mouse DENV model and to investigate the ability of the DIPs to block DENV transmission between vertebrates and mosquitos.

### Example 10 DIP Antivirals that Inhibit all DENV Serotypes in Vitro

Additional experiments will be conducted to determine the anti-DENV activity of different DIPs to DENV serotypes 1 to 4 compared to D2-290nt and D1-443nt. Human HuH7 and mosquito-derived C6/36 cell lines will be used for these experiments. The EC₅₀ value will be determined for each DIP against each DENV serotype. A DIP EC₅₀ is defined as the amount of DIP required to inhibit DENV replication in cell culture by 50%. Each cell type will be infected with DENV at an MOI 0.1 for 2 hours in triplicate. All DIPs used will be normalized for DI RNA content. Our experience with D2-290nt and D1-443nt DIPs has shown that 50 DI RNA copies per cell is sufficient to inhibit DENV replication by up to 98%. The DENV inoculums will be then replaced with untreated culture medium or medium treated with serially diluted DIPs. The levels of DI RNA and viral genomic RNA in both cell lysates and culture supernatant will be measured 5 days after infection. The level of infectious virus in culture supernatant by a viral plaque will also be measured after 5 days of infection. The level of infectious virus in culture supernatant by a viral plaque assay, will also be used to confirm antiviral activity of a DIP. Antiviral DIPs with the best EC50 will be used to generate a dose response curve.

### Example 11 Optimisation and Scaling Up DIP Production

DIPs will be produced by the Vero stable cell lines as described herein in a standard stationary culture, in a stirred culture using microcarrier beads (Mattos et al., 2015; Souza et al., 2009), and in a Wave Bioreactor. The readouts from each system will be DIP concentration as DI RNA copies/ml, which will be measured using supernatant clarified by low speed centrifugation that is filtered (0.22 μm) and then pelleted through a 20% Optiprep cushion by ultracentrifugation. This procedure removes cells and cellular debris. Preliminary results show that Vero-D2-Gen2 cells produce higher concentration DIPs in serum free medium VP-SFM than in any other medium tested. The production kinetics and concentrations of DIP will be compared between a 175 cm² flasks (˜50 ml), cells grown on cytodex 1 beads or cytodex 3 beads in stirred cultures and in a Wave Bioreactor. These systems increase the culture cell density by ˜4-fold compared to a stationary flask. Stirred cultures and the Wave system will be tested by seeding cells at 15% or 30% confluency (as recommended by the manufacturer) in 500 ml volume and transfecting cells with DI RNA using layered double hydroxide (LDH) nanoparticles (NP) (Wu et al., 2018), carrying 100 μg of DI RNA. LDH-NP transfection is a highly efficient RNA delivery method that has been established by our group. These will be added on day 1. Culture supernatant will be sampled every two days for 8 days, which will undergo ultracentrifugation in order to pellet DIPs. The pelleted DIPs will be assayed by RT-q PCR for DI RNA levels and by limiting dilution western blot for levels of DENV envelope protein using recombinant protein as a control. This will determine which culture system produces the highest concentration of DIP and the production kinetics, which will show the optimal time point for DIP harvest from culture supernatant. The best system will be scaled to 2 L, and our experience suggests that the total yield DIPs from 2 L should be >10¹⁰ DI RNA copies.

### Example 12 DIPs Purification and Analysis

The purification procedure described herein recovers >85% of total DIPs, removes exosome contamination and reduces contaminating proteins by 90% (Kurosawa et al., 2012). Briefly, DIP supernatants can be purified via i.) clarified by centrifugation, ii.) treated with benzonase to remove RNA and DNA, iii.) passed through a 0.22 μm membrane to remove cells and debris, iv.) hydroxyapatite chromatography using CHT type II resin (Kurosawa et al., 2012), v.) the eluted DIPs can be concentrated to ˜300 μL with a 100,000 MWCO centrifugal filter device (Richard et al., 2015), iv.) exchanged into storage buffer (pH 8.0) and stored at 4° C. DIPs are stable for weeks at 4° C. DIP purity can be determined by SDS-PAGE followed by Coomassie staining, western blot assays for DENV envelope and capsid proteins, nucleic acid staining and endotoxin contamination using a limulus amebocyte lysate assay. Total protein of a DIP preparation can be measured by the CBQCA protein quantification assay. DI RNA copy number can be measured by qRT-PCR. The combined assays will yield a complete biochemical profile for DIP preparations. If higher levels of purification are required, then concentrated DIPs can be further purified (>98% pure) using an OptiPrepTM velocity gradient (Rodenhuis-Zybert et al., 2010). The potency of a DIP preparation can be determined by measuring antiviral activity with respect to EC₅₀ units per ml of DIP preparation against each DENV serotype.

### Example 13 Determining if DENV Genome and DI RNA Co-Evolution Affects Virus Replication in Vitro

DENV and DI RNA compete for viral and cellular resources to achieve RNA replication. This competition may exert pressure that drives the virus to outcompete the DI RNA and vice-versa (escape from selection), resulting in co-evolution. While evolution of DI RNA in cells following a virus infection has been described, the co-evolution between virus and DI RNA has been hypothesised, but not formally investigated. Understanding whether co-evolution occurs and DIPs resistant DENV emerges is critical to the utility of the therapeutic DIPs. D2-290nt DIPs (Table 2), which exert strong negative pressure on the replication of DENV-2 will be used in this study. Over the course of the experiment, it will be assessed if the interplay between DENV-2 and D1-290nt DIP results in co-evolution of their RNA genomes and whether DIPs elicit a viral “resistance-proof” inhibition.

To assess this, HuH7 cells will be infected with virus produced using a plasmid-based infectious clone for DENV-2 (Rast et al., 2016) so that the virus stock is DIP-free. HuH7 cells will be incubated with virus equivalent to 100 copies of NS5 gene/cell for 2 h and then with D1-DI-443nt DIPs at a ratio of 1:1 and 1:10 (NS5 gene copy number to DI RNA copy number) overnight. If no virus replication is detected then the ratio of DENV:DIP genomes will be adjusted. UV-inactivated (Li et al., 2011) DIPs that lack antiviral activity will be used to confirm that DEN-2 virus inoculum used leads to robust virus replication. Culture supernatant (containing DENV and DIPs) will be transferred to uninfected HuH7 cells every 3 days for 10 passages. Passaging will be performed using 3 replicates. The culture supernatant collected at each passage will be used to measure virus titre by plaque assay, and DENV NS5 and DI RNA copy number by RT-qPCR. The diversity of virus and DI RNA sequence will be investigated by Illumina deep sequencing at passages 0 (start), passage 5 (middle) and passage 10 (end). For deep sequencing, RNA will be extracted from virus and DIPs purified from ˜½ of the culture supernatant collected, using QIAmp Viral RNA extraction kit. Viral RNA will be reverse transcribed into cDNA using DENV-2 and D2-DI-290nt specific primers. Libraries will be constructed with the Nextera kit and sequenced on a NextSeq at QIMRB core facilities, aiming for >3000 sequence depth at over 90% of bases. To look for adaptation during coevolution experiments, variants with a frequency >1% will be identified and tested to determine if the ratio of amino acid-changing to silent substitutions increases during coevolution. The increasing frequency of specific sequence variants during co-evolution experiments will be statistically tested to identify which DENV genomes and DI RNAs may be selected. If a key variant is identified, make mutant virus or DI RNA will be developed that contains the change/s to determine the effect on the dynamics of DENV and DI RNA replication.

TABLE 2
Purification of DIPs

| | DIP | | | | |
|---|---|---|---|---|---|
| | concentration | | | Protein | |
| Purification | (DI RNA | Total DIP yield | % | concentration | |
| step | copies/ml)(3) | (RNA copies) | recovery(4) | (mg/ml)(5) | EC50(6) |
| 0.2 μm filter | 1 × 10⁷ | 3.7 × 10⁸ | 100 | 3.19 3.2 ± 1.4 | 3.2 ± 1.4 |
| CHT column (1) | 3.2 × 10⁸ | 3.0 × 10⁸ | 80 | 0.28 | 0.27 ± 0.05 |
| Centricon (2) | 9.9 × 10⁹ | 2.8 × 10⁸ | 76 | 0.37 | 0.08 ± 0.002 |

1. Ceramic hydroxyapatite chromatography as described (Rodenhuis-Zybert I A et al. (2010) PLoS PAthog 6, e1000718)

2. 100K molecular weight cut-off

3. RNase-resistant DI RNA measured by RT-qPCR assay

4. Recovery of DI RNA relative to unfiltered supernatant

5. Bradford assay

6. The volume (pl) of DIP preparation required to reduce DENV titre (plaque form units, PFU) by 50% after 24 h. Vero cells grown in 24 well plates are incubated with DENV (MOI 0.1) for 2 h, virus is removed and new medium lacking DIPs or with serially diluted DIP is added. All assays are performed in triplicate. The mean value and SD is shown.

### Example 14 Pre-Clinical Evaluation of DIPs Safety and Antiviral Activity in a DENV Mouse Model

An B6 interferon α^{-/-}β^{-/-}R (IFNR1) knockout (KO) mouse model of dengue infection will be used to investigate antiviral activity of DI RNAs in vivo (Orozco et al 2012; Prestwood et al., 2012). QIMR Berghofer houses a colony of these mice in its mouse house which are used to evaluate DENV infection and anti-DENV agents. Viremia is detected in plasma that peaks in 4-5 days and is measurable for ˜7 days. Mice experience a non-lethal acute DENV infection which makes the model suitable for analysis of DIP inhibition of DENV replication. Male and female adult IFNR1 mice will be used as virus infection is not gender biased. Initially, the mouse adapted DENV-2 strain D220 will be used to assess safety of DIPs in mice, treatment of DENV-2 infected mice with D2-290nt and D1-443nt DIPs and the time of DIPs administration.

### Safety of DIPs in Mice

B6 WT or B6 IFNR1 mice will be injected i.v. with a range of DIP D2-290nt concentrations (106, 107, and 108 DI RNA copies in 100 μL). UV irradiated inactive DIPs (Dimmock and Easton, 2014) or blank storage buffer will be used as negative controls. Twelve mice will be used for each group. Mice will be weighed and scored daily for morbidity. Six mice from each treated group will be sacrificed after 3 and 6 days and blood, liver, large intestine, kidney, spleen and lung will be collected for tissue morphology analysis. Tissue sections will be made for histopathology analysis of treated and untreated mice by hematoxylin and eosin (H & E) staining to examine if DIP treatment affect tissue morphology. The mice will be sacrificed immediately if a maximum morbidity score is recorded. Blood chemistry including the electrolytes (sodium, potassium, calcium, chloride, inorganic phosphate), lipids (cholesterol, triglyceride), and enzyme activities (ALT, AST, ALP, a-amylase) and urea, albumin, and total protein levels will be checked.

Treatment of DENV-2 Infected Mice with D2-290nt and D1-443nt DIPs

Six B6 IFNR1 mice will be used for each DENV titre tested. The mouse adapted DENV-2 D220 strain will be used at 10³, 10⁴ and 10⁵ (a non-lethal maximum dose) plaque forming units (p.f.u.) (Orozco et al., 2012). The maximum tolerated dose of DIPs (3.1 above), inactivated DIPs, or storage buffer will be mixed with virus stock and mice will be i.v. injected. Viremia will be monitored for 7 days in EDTA-treated plasma samples collected daily and processed for measurement of DI RNA and viral genomic RNA by qRT-PCR, and for infectious DENV by plaque assay. Mice will be weighed and scored daily for morbidity. Mice that have a maximum morbidity score or after 7 days post infection will be humanely sacrificed, when organs (liver, kidney, spleen and large intestine) will be collected for H & E analysis of tissue sections to check morphology for evidence of disease and tissue RNA will be collected for measurement of DI RNA and viral genomic RNA levels by RT-qPCR. In follow-up experiments, the concentrations of DIP will be titrated up or down, depending on outcomes, in order to determine if virus infection is regulated by DIP treatment in a dose dependent manner.

### Timing of DIP Administration

Experiments will be performed that mimic different circumstances of DENV infections where DIP administration may occur at different stages of infection; i.e. prophylactic administration prior to infection or therapeutic administration post infection. DIPs, inactivated DIPs or storage buffer will be administered at 6 h or 24 h pre-infection and post-infection and the viremia will be monitored as using animal numbers, sampling and analysis as described in the preceding paragraph.

### DENV-1-3, -4 Infection Kinetics in IFNR1 Mice

Mice will be infected with DENV serotype 1, 3 and 4 that result in acute viremia and the ability of DIPs to inhibit each DENV serotype will be assessed. Using groups of 3 mice, DENV infection kinetics in IFNR1 mice will be established i.v. injection of previously described DENV serotype-1 Mochizuki strain at 10⁶ p.f.u., serotype-3 C0360/94 strain at 10⁷ p.f.u.- and serotype-4 TVP-376 strain at 10⁷ p.f.u. (Hotta, 1952; Sarathy et al., 2018; Sarathy et al., 2015). Each day plasma samples will be collected; the mice will be weighed and scored for morbidity. The objective will be a reproducible viremia measurable in plasma at 10⁴-10⁷ genomic equivalents RNA/ml and minimal signs or symptoms of infection. Depending on viremia levels in plasma, the virus inoculum will be adjusted and the experiment will be repeated. An animal will be sacrificed if a maximum morbidity score is reached, and no later than 7 days post-infection when organs will be collected for analysis of viral RNA. An individual DIP with strong in vitro antiviral activity to each DENV serotype will be tested in IFNR1 mice infected as described above but with DENV-1, -3, and -4 serotypes.

### Example 15 Mosquito Transmission Models: Attenuation of DENV Transmission by DIPs

Mosquitoes are essential components of the DENV transmission cycle. As it requires 10⁴˜10⁶ virions to infect a mosquito, reductions of even 1 or 2 logs by DIP treatment may reduce the titre to below this threshold, effectively blocking onward transmission. Mosquito-mouse model procedures (Hugo et al., 2016) and entomological procedures will be employed to investigate how DIPs alter DENV transmission dynamics to mosquitoes.

### Investigation of DENV Infection and Replication in Mosquitos

This will initially be assessed using D1-443nt and D2-290nt DIPs, but will be expanded to include additional DI RNAs. The inventors have established a DENV transmission model using an artificial membrane feeding apparatus (Kho et al. 2016). Infectious DNA clones will be used to make DENV-1 and -2 virus stocks that are DIP-free. To infect mosquitoes, blood meals containing ˜10⁷ plaque forming units/ml of DENV will be mixed with DIPs equivalent to 0, 10⁸, 10⁹ and 10¹⁰ D1-443nt or D2-290 copies/ml. The DIP:DENV mixtures will be fed to Ae. aegypti mosquitoes in blood meals using the feeding apparatus. Typically, 80 mosquitoes are fed per feeder and ˜75% are infected. Engorged mosquitoes will be incubated at 28° C., 75% relative humidity for 14 days before being harvested, bodies, legs/wings and saliva assayed for DENV-2 by a 50% infectious dose cell culture ELISAs (CCELISAs) as previously described (Hugo et al., 2019), and for the levels of DI RNA by RT-qPCR assay. DI RNAs will be sequenced to verify that the original DI RNAs are maintained. This experiment will indicate (a) whether the DIP has reduced the proportion of infected mosquitoes or the titre of virus in their tissues and (b) whether the DIP or DENV has reached the mosquito saliva, which is required to make transmission possible.

Determining Transmission of DENV from DIP Treated Mice to Mosquitoes

Transmission of DENV to mosquitoes will be investigated using infected mice as described above. Three sets of 6 mice; DENV+DIPs, DENV+inactivated DIP, and DENV injected with storage buffer only will be used in this study. Then, cups of 20 naïve mosquitoes will be allowed to feed on mice daily during the viraemic period (3-6 days post-infection) (Christofferson et al., 2010). The mosquitoes (1440 in total) will be incubated as described in section 4.1 and harvested at 14 days after mouse feeding. The bodies, legs and wings and saliva will be tested to determine the percentage infection and titre of live virus by CCELISA. A reduction to the mosquito infection and dissemination into tissue and saliva will indicate if DIPs reduce infection of mosquitoes via blood feeding.

### Example 16 D2-290nt DI RNA Reduces ZIKAV Genomic RNA in Infected Cells

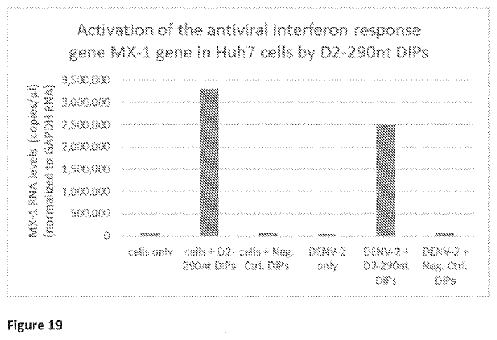

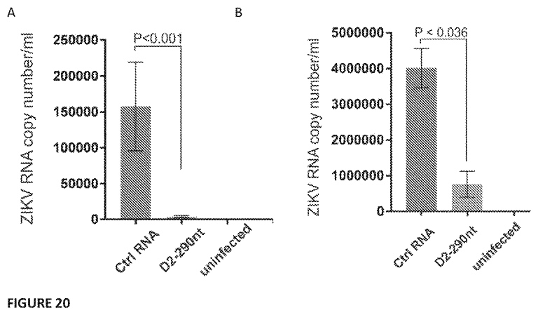

Based on the above data, it was confirmed that DENV DI RNA can inhibit replication of other Flaviviruses such as ZIKV. This is because Flaviviruses share RNA structure homology in the 5′ and 3′ RNA UTRs that regulates virus RNA replication (Ng et al., 2017). DIPs and their DI RNA DIPs may stimulate innate cellular antiviral pathways such as interferon response genes such as MX-1 (FIG. 19), which is a powerful way to inhibit RNA viruses. To test this possibility, we delivered the D2-290nt DI RNA or a control RNA that lacks antiviral activity to human Huh7 cells in triplicate and then infected with Zika virus (ZIKV) (MOI of 0.1 or 0.01) for 3 hours (FIG. 20). Uninfected Huh7 was included as a negative control. HuH7 cells are often used to investigate replication of ZIKV (Vicentiet al., 2018). Supernatant samples were collected after 3 days post infection and assayed for levels of ZIKV genomic RNA by RT-qPCR in triplicate. The level of ZIKV genomic RNA in supernatant from D2-290nt-treated cells was reduced by ˜5-fold when infected at an MOI of 0.1 and by ˜39-fold when an MOI of 0.01 was used compared to control-RNA-treated cells. A Student's t-test P valu e showed that reduced viral genome levels by D2-290nt compared to the Ctrl RNA was significant.

This result provides strong evidence that DENV DIPs have potential to inhibit replication of different members of the Flavivirus genus.

### Example 17 Statistical Analysis

One-way or two-way ANOVAs, or regressions analysis will be used as appropriate, followed by t-tests to assess specific comparisons of interest. Sample size is balanced between animal ethics and statistical rigour. With 6 mice per group experiments have 80% power for level α=0.05 test comparing two groups if they differ by 1.6 standard deviations. In mouse models, experimental controls result in relatively small standard deviations and large effects of this size are expected.

### Remarks

The results herein demonstrate that DENV defective interfering particles (DIP) can be produced which are free of infectious reconstituted virus, are transmissible and can inhibit DENV-1 and DENV-2 and potentially other members of the Flaviviridae family.

The present inventors have established in vitro system for the production of potent anti-dengue virus DIP. The system utilises a stable cell line that produces all of the DENV structural and non-structural proteins in the absence of live virus. This cell line supports the replication of naturally occurring defective interfering RNAs identified from DENV infected patients and packages them into DIPs in high quantities. The DIPs are described herein are suitable for the treatment and/or prevention of Flaviviridae, reducing the viral load of Flaviviridae, and can be useful for reducing the transmission of Flaviviridae between a host and a carrier.

The DIPs have certain advantages including high specificity, broad anti-viral activity against a range of viruses and serotypes, and the ability to block or attenuate virus transmission.

### REFERENCES

Ansarah-Sobrino, Temperature-dependent production of pseudoinfectious dengue reporter virusparticles by complementation. Virol 381, 67-74 (2008).

Ausubel, (Ed.). Current Protocols in Molecular Biology. New York: John Wiley & Sons. (1987 including all updates untill present).

Barrett and Dimmock, Defective interfering viruses and infections of animals. Curr Top Microbiol Immunol 128, 55-84 (1986).

Buchschacher and Panganiban. Human immunodeficiency virus vectors for inducible expression of foreign genes. J Virol, 2731-2739 (1992).

Burns et al., Vesicular stomatitis virus G glycoprotein pseudotyped retroviral vectors: concentration to very high titer and efficient gene transfer into mammalian and nonmammalian cells. Proceedings of the National Academy of Sciences USA, 8033-8037 (1993).

Carter, Adeno-associated virus vectors. Current Opinion in Biotechnology, 533-539 (1992).

Christofferson et al., Development of a transmission model for dengue virus. Virol J 10, 127 (2013).

Dimmock and Easton, Defective interfering influenza virus RNAs: time to reevaluate their clinical potential as broad-spectrum antivirals? J Virol 88, 5217-5227 (2014).

Fisher-Hoch et al., Protection of rhesus monkeys from fatal Lassa fever by vaccination with a recombinant vaccinia virus containing the Lassa virus glycoprotein gene. Proceedings of the National Academy of Sciences USA, 56, 317-321 (1989).

Hotta, Experimental studies on dengue. I. Isolation, identification and modification of the virus. J Infect Dis 90, 1-9 (1952).

Hugo et al., Chikungunya virus transmission between Aedes albopictus and laboratory mice. Parasit Vectors 9, 555 (2016).

Hugo et al., Vector competence of Australian Aedes aegypti and Aedes albopictus for an epidemic strain of Zika virus. PLoS Negl Trop Dis 13, e0007281 (2019).

Johann et al., GLVR1, a receptor for gibbon ape leukemia virus, is homologous to a phosphate permease of Neurospora crassa and is expressed at high levels in the brain and thymus. J Virol, 65, 1635-1640 (1992).

Kho et al., Effects of Larval Nutrition on Wolbachia-Based Dengue Virus Interference in Aedes aegypti (Diptera: Culicidae). J Med Entomol 53, 894-901 (2016).

Kotin, Prospects for the Use of Adeno-Associated Virus as a Vector for Human Gene Therapy, Human Gene Therapy, 793-801 (1994).

Kurosawa et al., Purification of Dengue Virus Particles by One-Step Ceramic Hydroxyapatite Chromatography. World J Vaccines 2, 155 (2012).

Lebkowski et al., Adeno-associated virus: a vector system for efficient introduction and integration of DNA into a variety of mammalian cell types. Mole and Cell Biology, 3988-3996 (1988).

Li et al., Defective interfering viral particles in acute dengue infections. PLoS One 6, e19447 (2011).

Li et al., Sub-genomic RNA of defective interfering (D.I.) dengue viral particles is replicated in the same manner as full length genomes. Virology 468-470:248-255 (2014).

Mattos et al., Increasing Vero viable cell densities for yellow fever virus production in stirred-tank bioreactors using serum-free medium. Vaccine 33, 4288-4291 (2015).

Miller and Rosman. Improved Retroviral Vectors for Gene Transfer and Expression. Biotechniques, 7, 980-990 (1989).

Miller, Retrovirus Packaging Cells. Human Gene Therapy, 7, 5-14 (1990).

Miller et al., Construction and properties of retrovirus packaging cells based on gibbon ape leukemia virus. J Virol, 65, 2220-2224 (1991).

Muzyczka, Use of adeno-associated virus as a general transduction vector for mammalian cells. Current Topics in Microbiology and Immunology, 158, 97-129 (1992).

Ng et al., The 5′ and 3′ Untranslated Regions of the Flaviviral Genome, Viruses, 9, (2017).

Orozco et al., Characterization of a model of lethal dengue virus 2 infection in C57BL/6 mice deficient in the alpha/beta interferon receptor. J Gen Virol 93, 2152-2157 (2012).

Prestwood et al., Gamma interferon (IFN-gamma) receptor restricts systemic dengue virus replication and prevents paralysis in IFN-alpha/beta receptor-deficient mice. J Virol 86, 12561-12570 (2012).

Rast et al., Conflicting Selection Pressures Will Constrain Viral Escape from Interfering Particles: Principles for Designing Resistance-Proof Antivirals. PLoS Comput Biol 12, e1004799 (2016).

Richard et al., Use of Centrifugal Filter Devices to Concentrate Dengue Virus in Mosquito per os Infection Experiments. PLoS One 10, e0138161 (2015).

Rodenhuis-Zybert et al., Immature dengue virus: a veiled pathogen? PLoS Pathog 6, e1000718 (2010).

Salas-Benito and De Novo-Ocampo, Viral Interference and Persistence in Mosquito-Borne Flaviviruses. J Immunol Res, 873404 (2015)

Sarathy et al., Characterization of lethal dengue virus type 4 (DENV-4) TVP-376 infection in mice lacking both IFN-alpha/beta and IFN-gamma receptors (AG129) and comparison with the DENV-2 AG129 mouse model. J Gen Virol 96, 3035-3048 (2015).

Sarathy et al., Characterization of a murine model of non-lethal, symptomatic dengue virus infection. Sci Rep 8, 4900 (2018).

Scarpa et al., Characterization of recombinant helper retroviruses from Moloney-based vectors in ecotropic and amphotropic packaging cell lines. Virology, 75, 849-852 (1991).

Sommerfelt and Weiss, Receptor interference groups of 20 retroviruses plating on human cells. Virol, 76, 58-59 (1990).

Souza et al., Production of yellow fever virus in microcarrier-based Vero cell cultures. Vaccine 27, 6420-6423 (2009).

Verghese et al., S/MAR sequence confers long-term mitotic stability on non-integrating lentiviral vector episomes without selection. Nucleic Acids Res 42(7) (2014).

Vicenti et al., Comparative analysis of different cell systems for Zika virus (ZIKV) propagation and evaluation of anti-ZIKV compounds in vitro. Virus Res, 244, 64-70 (2018).

Vincent et al., Replication and Packaging of HIV Envelope Genes in a Novel Adeno-associated Virus Vector System. Vaccine, 353-359 (1990).

Wilson et al., Formation of infectious hybrid virions with gibbon ape leukemia virus and human T-cell leukemia virus retroviral envelope glycoproteins and the gag and pol proteins of Moloney murine leukemia virus. J Virol, 63, 2374-2378 (1989).

Wu et al., Optimization of Formulations Consisting of Layered Double Hydroxide Nanoparticles and Small Interfering RNA for Efficient Knockdown of the Target Gene. ACS Omega 3, 4871-4877 (2018).

Wolf et al. (2008) Chem Eng Technol. 31(6):846-867.

Wolf et al. (2011) Expert Rev Vaccine. 10 (10): 1451-1475.

Josefsberg et al. (2012) Biotech and Bioengineering. 109(9)1443-1460.

Kalbfuss et al (2006). Biotech and Bioengineering. 97(1):73-85.

1. A cell line for producing virus defective interfering particles (DIPs), comprising:
(i) a first vector for expression of the non-structural proteins of a virus of the Flaviviridae family;
(ii) a second vector for expression of the structural proteins of a virus of the Flaviviridae family (i); and
wherein, upon the introduction of a third vector for the expression of a Flaviviridae defective interfering genomic sequence the cell produces DIPs.

2. The cell line according to claim 1, wherein the virus of the Flaviviridae family of (i) and (ii) are the same virus.

3. The cell line according to claim 1, wherein the virus of the Flaviviridae family of (i) and (ii) are not the same virus.

4. The cell line according to any one of claims 1 to 3, wherein the DIP is capable of only a single round of infection.

5. The cell line according to any one of claims 1 to 4, wherein the virus defective interfering genomic sequence is modified relative to the genomic sequence of its corresponding infectious native viral genomic sequence.

6. The cell line according to any one of claims 1 to 5, wherein the virus defective interfering genomic sequence does not include the genes encoding viral structural and non-structural proteins.

7. The cell line according to any one of claims 1 to 6, wherein the virus defective interfering genomic sequence comprises about 3 to 10% of the total viral genomic sequence relative to the corresponding native virus.

8. The cell line according to any one of claims 1 to 7, wherein the virus defective interfering genomic is expressed and packaged as RNA.

9. The cell line according to any one of claims 1 to 8, wherein the defective interfering genomic sequence is selected from the group comprising or consisting of any one of SEQ ID NO:26 to SEQ ID NO:41.

10. The cell line according to any one of claims 1 to 9, wherein the cell line comprises:
(i) a first vector for expression of the non-structural proteins of a virus of the Flaviviridae family;
(ii) a second vector for expression of the structural proteins of a virus of the Flaviviridae family (i); and
(iii) a third vector for the expression of a Flaviviridae defective interfering genomic sequence.

11. The cell line according to any one of claims 1 to 10, wherein the non-structural proteins comprise one or more, or all of NS1, NS2A, NS2B, NS3, NS4A, NS4B and NS5.

12. The cell line according to any one of claims 1 to 11, wherein the structural proteins comprise one or more or all of capsid (C), pre-membrane/membrane (prM), and envelope (E).

13. The cell line according to any one of claims 1 to 12, wherein the third vector comprises a Flaviviridae defective interfering genomic sequence

14. The cell line according to any one of claims 1 to 13, wherein the first, second and third vectors are retroviral or lentiviral vectors or a combination thereof.

15. The cell line according to any one of claims 1 to 14, wherein one or more of the first, second, and third vectors are self-inactivating (SIN) vectors.

16. The cell line according to one of claims 1 to 15, wherein the structural proteins and/or non-structural proteins are human and/or Old World monkey codon optimised.

17. The cell line according to any one of claims 1 to 16, wherein introduction of the third vector into the cell line is by transfection or transduction.

18. The cell line according to any one of claims 1 to 17, wherein the defective interfering genomic sequence is constitutively expressed in the cell.

19. The cell line according to any one of claims 1 to 18, wherein the DIPs are continuously secreted from the cell.

20. The cell line according to any one of claims 1 to 19, wherein the defective interfering genomic sequence comprises about 155 nucleotides to about 1000 nucleotides.

21. The cell line according to any one of claims 1 to 20, wherein the defective interfering genomic sequence comprises about 200 nucleotides to about 500 nucleotides.

22. The cell line of any one of claims 1 to 21, wherein the Flaviviridae is selected from: Flavivirus, Hepacivirus, Pegivirus, Pestivirus, and Jingmenvirus.

23. The cell of claim 22, wherein the Flavivirus is selected from the group consisting of: Dengue virus (DENV), West Nile virus (WNV), Yaounde virus, Yellow fever virus (YFV), Zika virus (ZIKA), Apoi virus, Aroa virus, Bagaza virus, Banzi virus, Bouboui virus, Bukalasa bat virus, Cacipacore virus, Carey Island virus, Cowbone Ridge virus, Dakar bat virus, Edge Hill virus, Entebbe bat virus, Gadgets Gully virus, Ilheus virus, Israel turkey meningoencephalomyelitis virus, Japanese encephalitis virus, Jugra virus, Jutiapa virus, Kadam virus, Kedougou virus, Kokobera virus, Koutango virus, Kyasanur Forest disease virus, Langat virus, Louping ill virus, Meaban virus, Modoc virus, Montana myotis leukoencephalitis virus, Murray Valley encephalitis virus, Ntaya virus, Omsk hemorrhagic fever virus, Phnom Penh bat virus, Powassan virus, Rio Bravo virus, Royal Farm virus, Saboya virus, Sal Vieja virus, San Perlita virus, Saumarez Reef virus, Sepik virus, St. Louis encephalitis virus, Tembusu virus, Tick-borne encephalitis virus, Tyuleniy virus, Uganda S virus, Usutu virus, Wesselsbron virus, and Yokose virus.

24. The cell line of claim 22 or 23, wherein the Flavivirus is selected from DENV, ZIKA, WNV, and YFV

25. The cell line according to any one of claims 22 to 24, wherein the DENV has a serotype selected from one or more of DENV1, DENV2, DENV3, and DENV4.

26. A method for producing virus defective interfering particles (DIPs), comprising transfecting or transducing the cell line according to any one of claims 1 to 25 with a vector comprising a Flaviviridae defective interfering genomic sequence according to any one of claims 5 to 9, wherein the cell line comprises (i) a first vector which expresses the non-structural proteins of a virus of the Flaviviridae family; and (ii) a second vector which expresses the structural proteins of the same virus according to (i); and wherein when the Flaviviridae defective interfering genomic sequence is expressed in the cell line by a third vector, the cell line produces DIPs.

27. A method for producing virus defective interfering particles (DIPs), comprising expressing a Flaviviridae defective interfering genomic sequence according to any one of claims 5 to 9 in a cell line comprising i) a first vector which expresses the non-structural proteins of a virus of the Flaviviridae family; and (ii) a second vector which expresses the structural proteins of the same virus according to (i); and wherein when the Flaviviridae defective interfering genomic sequence is expressed in the cell line by a third vector, the cell line produces DIPs.

28. A cloned or recombinant virus defective interfering particle (DIP) expressed by the cell line of any one of claims 1 to 25, or produced by the method of claim 26 or 27.

29. An isolated virus defective interfering particle (DIP) or a population of DIPs expressed by the cell line according to any one of claims 1 to 25, or produced by the method of claim 26 or 27.

30. A pharmaceutical composition comprising the DIP of claim 28 or 29.

31. An immunogenic composition comprising the DIP of claim 28 or 29.

32. A method of treating or preventing a Flaviviridae disease comprising administering to a subject in need thereof the DIP according to claim 28 or 29, the pharmaceutical composition of claim 30, or the immunogenic composition of claim 31.

33. A method of reducing the load of a Flavivirus RNA in a subject comprising administering to the subject the DIP according to claim 28 or 29, the pharmaceutical composition of claim 30, or the immunogenic composition of claim 31.

34. A method of reducing transmission of a Flaviviridae between a Flaviviridae host and a Flaviviridae carrier comprising administering the DIP according to claim 28 or 29, the pharmaceutical composition of claim 30, or the immunogenic composition of claim 31.

35. A method of reducing transmission of a Flaviviridae between a Flaviviridae host and a Flaviviridae carrier comprising administering the DIP according to 28 or 29, the pharmaceutical composition of claim 30, or the immunogenic composition of claim 31.

36. Use of a virus defective interfering particle (DIP) according to 28 or 29 in the manufacture of a medicament for treating or preventing a Flaviviridae disease in a subject.

37. Use of a virus defective interfering particle (DIP) according to 28 or 29 in the manufacture of a medicament for reducing the load of an RNA virus in a subject.

38. A vector comprising a Dengue virus defective interfering genomic sequence encoding a Dengue virus interfering RNA sequence, wherein the vector is capable of inhibiting replication by a wild-type Dengue virus in a cell or a host when the vector is introduced into the cell or host.

39. A nucleic acid sequence encoding a Dengue virus defective interfering RNA sequence, wherein the sequence is capable of inhibiting replication by a wild-type Dengue virus in a cell or a host infected with the Dengue virus comprising administering to the cell or host a sequence selected from SEQ ID NO:26 to SEQ ID NO:41.
